(19) United States
(12) Patent Application Publication
(10) Pub. No.: US 2024/0150829 A1
(43) Pub. Date: May 9, 2024

(54) SYSTEM AND METHODS OF DETECTION OF ONCRNAS FOR CANCER DIAGNOSIS

(71) Applicant: The Regents of the University of California, Oakland, CA (US)

(72) Inventor: Hani GOODARZI, San Francisco, CA (US)

(21) Appl. No.: 18/110,785

(22) Filed: Feb. 16, 2023

Related U.S. Application Data
(63) Continuation of application No. PCT/US2021/046186, filed on Aug. 16, 2021.
(60) Provisional application No. 63/066,269, filed on Aug. 16, 2020.

(51) Int. Cl. C12Q 1/6874; C12Q 1/48; C12Q 1/6806; C12Q 1/6844

(52) U.S. Cl. CPC C12Q 1/6874; C12Q 1/485; C12Q 1/6806; C12Q 1/6844; G01N 2333/9128

(57) ABSTRACT

The present disclosure relates generally to detection of non-coding RNA molecules in a sample or diagnosis of a subject based upon detection or quantification of non-coding RNA molecules in a sample of the subject, specifically to identify and use of molecular biomarkers for cancer diagnosis.

### CROSS-REFERENCE TO RELATED APPLICATIONS

The present application is a United States non-provisional continuing application filed under 35 U.S.C. § 111 and claiming priority to International Application No. PCT/US2021/046186, filed on Aug. 16, 2021, which claims priority to U.S. Provisional Patent Application No. 63/066,269, filed Aug. 16, 2020, the contents of each of which are hereby incorporated by reference in their entireties.

### SUBMISSION OF SEQUENCE LISTING

The Sequence Listing associated with this application is filed in electronic format via Patent Center and is hereby incorporated by reference into the specification in its entirety. The name of the text file containing the Sequence Listing is UCAL018USSeqListing.xml. The size of the text file is 16.2 MB and was created on Mar. 27, 2023.

### TECHNOLOGY FIELD

The present disclosure relates generally to detection of non-coding RNA molecules in a sample or diagnosis of a subject based upon detection or quantification of non-coding RNA molecules in a sample of the subject, specifically to identify and use of molecular biomarkers for cancer diagnosis.

### BACKGROUND

The widespread reprogramming of the gene expression landscape is a hallmark of cancer development. Thus, the systematic identification of regulatory pathways that drive pathologic gene expression patterns is a crucial step towards understanding and treating cancer. Over the years, a multitude of regulatory mechanisms have been implicated in oncogenic expression of genes involved in cancer cell differentiation, survival, invasion, and spread. While numerous studies have focused on the transcriptional pathways that underlie oncogenesis, post-transcriptional regulatory pathways have also emerged as major regulators of this process. For example, microRNAs (small non-coding RNAs), a subclass of small RNAs that function in gene silencing, were among the first characterized post-transcriptional regulators of breast cancer progression (1). RNA-binding proteins (RBPs) are also critical posttranscriptional regulators of gene expression, and several specific RBPs have been shown to affect oncogenesis and cancer progression (2-5). Recently, it was demonstrated that transfer RNAs (tRNAs, 6) and tRNA fragments (7), which are other classes of small non-coding RNAs, play a fundamental role in breast cancer progression.

Despite the diverse repertoire of regulatory mechanisms involved in cancers, a shared characteristic among them is that they co-opt and dys-regulate existing pathways within the cell. In other words, cancer cells adopt myriad strategies, such as somatic mutations (e.g. KRAS, 8), gene fusions (e.g. BCR-ABL, 9), epigenetic modifications (e.g. promoter hypermethylation, 10), and regulatory mechanisms disruptions (NFkB transcription factors, 10) to over-activate oncogenic and to down-regulate tumor suppressive pathways (11, 12). While these strategies rely on the pathologic modulation of regulatory programs that are already in place, there is an often-overlooked possibility that cancer cells may be capable of evolving or engineering specialized regulatory pathways that drive tumorigenesis.

### SUMMARY

Performing a systematic screen for small non-coding RNAs that are expressed by breast cancer but are absent from healthy normal, a set of 201 small non-coding RNAs were previously identified as being strongly associated with breast cancer but mostly undetectable in normal cells. Because these small non-coding RNA are cancer-associated but functionally unknown, they have been named “orphan non-coding RNAs” or “oncRNAs.” This prior finding and the applications of those oncRNAs in diagnosis and/or detection of breast cancer has been previously described in WO 2019/094780 published on May 16, 2019 and Fish et al., Nat. Med., 2018, 24:1743-1751, the content of each publication is incorporated herein by reference in its entirety.

The present disclosure provides novel small non-coding RNAs that serve as biomarkers which are indicative of various types of cancer, tissue of origin, and status of the cancer, and which may be used to accurately diagnose cancer in a subject even at an early stage. In some embodiments, the methods comprise detection of extracellular, circulating small non-coding RNAs in a suitable sample. In some embodiments, the sample is a human serum sample. In some embodiments, the sample is a fractionated human serum sample comprising exosomes that comprise small non-coding mRNA.

The disclosure relates to a method of diagnosing a subject with a benign, pre-malignant, or malignant hyperproliferative cell, said method comprising detecting the presence, absence, and/or quantity of at least one non-coding RNA or functional fragment thereof in a sample of the subject. In some embodiments, the step of detecting in the method of the disclosure is preceded by a step of acquiring the sample from the subject. In some embodiments, the step of detecting the presence, absence, and/or quantity of at least one non-coding RNA or functional fragment thereof in the sample comprises contacting the sample with one or a plurality of probes specific for the at least one non-coding RNA or functional fragment thereof, and normalizing the quantity in the sample with a measurement taken from a control sample.

In some embodiments, the disclosed method further comprises exposing the sample to at least one nucleic acid molecule complementary to one or a plurality of non-coding RNAs chosen from SEQ ID NO: 1 through SEQ ID NO: 1543, or one or a plurality of non-coding nucleic acid sequences that comprise at least about 70%, 80%, 85%, 90%, 95%, 96%, 97%, 98% or 99% sequence identity to any of nucleic acids chosen from SEQ ID NO: 1 through SEQ ID NO: 1543. In some embodiments, the disclosed method further comprises correlating the amount of the at least one non-coding RNA or functional fragment thereof in the sample to the probability or likelihood the subject has a benign, pre-malignant, or malignant hyperproliferative cell growth, relative to a measurement of the amount of the at least one non-coding RNA or functional fragment thereof in a control sample.

In some embodiments, the benign, pre-malignant, or malignant hyperproliferative cell diagnosed by the disclosed method is from a tissue chosen from: adrenal gland, bile ducts, bladder, blood, bone, bone marrow, brain, breast, cervix, colon, esophagus, eye, gall bladder, ganglia, gastrointestinal tract, heart, lymphatic system, liver, lung, kidney, muscle, ovary, pancreas, parathyroid, penis, prostate, prostate glands, rectum, salivary glands, skin, spine, stomach, spleen, testis, thymus, thyroid, or uterus. In some embodiments, the disclosed method diagnoses the presence of a pre-malignant or malignant hyperproliferative cell in the subject chosen from one or a plurality of basal or luminal cancers.

In some embodiments, the sample is blood or serum from the subject. In some embodiments, the sample is taken from a culture of cells seeded or inoculated by at least one cell from the subject. In some embodiments, the sample is taken from a culture of cells seeded or inoculated by at least one primary cell from the subject. In some embodiments, the sample is a human tissue sample comprising a tissue or liquid sample from a plasma, serum or blood draw, brushing, biopsy, or surgical resection of the subject. In some embodiments, the sample comprises a cell that is freshly obtained, formalin fixed, alcohol-fixed and/or paraffin embedded.

In some embodiments, the disclosed method further comprises culturing at least one biopsy from the subject with a culture medium under conditions and for a time period sufficient to grow at least one cell from the subject's tissue chosen from: adrenal gland, bile ducts, bladder, blood, bone, bone marrow, brain, breast, cervix, colon, esophagus, eye, gall bladder, ganglia, gastrointestinal tract, heart, lymphatic system, liver, lung, kidney, muscle, ovary, pancreas, parathyroid, penis, prostate, prostate glands, rectum, salivary glands, skin, spine, stomach, spleen, testis, thymus, thyroid, or uterus. In some embodiments, the sample is free of oncRNAs from breast cancer cells. In some embodiments, the subject is free of a hyperproliferative cell from the breast or has not been diagnosed with or treated for breast cancer.

In some embodiments, the step of measuring the quantity of the at least one non-coding RNA or functional fragment thereof in the sample in the disclosed method comprises one or a combination of: digitally imaging the sample, exposing the sample to a known amount of labeled antibody specific for an epitope of the at least one non-coding RNA or functional fragment thereof, exposing the sample to one or a plurality of dyes specific for the at least one non-coding RNA or functional fragment thereof, exposing the sample to at least one labeled probe comprising a nucleotide sequence complementary to a sequence of the at least one non-coding RNA or functional fragment thereof, exposing the sample to chromatography, isolating total RNA of the sample and exposing the total RNA to sequencing analysis and/or exposing the sample to mass spectrometry. In some embodiments, this step further comprises analyzing morphology of cells from the sample.

In some embodiments, the step of detecting the presence, absence, and/or quantity of the at least one non-coding RNA or functional fragment thereof in the sample of the disclosed method comprises using a chemoluminescent probe, fluorescent probe, and/or fluorescence microscopy. In some embodiments, this step of detecting further comprises contacting total RNA of the sample to at least one probe comprising s nucleotide sequence complementary to one or a plurality of non-coding RNAs chosen from SEQ ID NO: 1 through SEQ ID NO: 1543, or one or a plurality of non-coding nucleic acid sequences that comprise at least about 70%, 80%, 85%, 90%, 95%, 96%, 97%, 98% or 99% sequence identity to any of nucleic acids chosen from SEQ ID NO: 1 through SEQ ID NO: 1543.

The disclosure further relates to a method of detecting a cancer cell in a subject comprising detecting the presence, absence, and/or quantity of at least one non-coding RNA or functional fragment thereof in a sample by contacting the sample with one or a plurality of probes comprising nucleotide sequences complementary to the at least one non-coding RNA sequences. In some embodiments, the step of detecting is preceded by a step of obtaining the sample from the subject. In some embodiments, the disclosed method further comprises:
a) calculating one or more scores based upon the presence, absence, or quantity of the at least one non-coding RNA or functional fragment thereof; and
b) correlating the one or more scores to the presence, absence, or quantity of the at least one non-coding RNA or functional fragment thereof, such that, if the amount of the at least non-coding RNA or functional fragment thereof is greater than the quantity of the at least one non-coding RNA or functional fragment thereof in a control sample; or, if the amount of the at least one non-coding RNA or functional fragment thereof is substantially equal to the quantity of the at least one non-coding RNA or functional fragment thereof in a sample taken from a subject known to have cancer then the subject is diagnosed as having cancer.

In some embodiments, the at least one non-coding RNA or functional fragment thereof detected by the disclosed method comprises at least about 70%, 80%, 85%, 90%, 95%, 96%, 97%, 98%, 99% sequence identity to any of SEQ ID NO: 1 through SEQ ID NO: 1543. In some embodiments, the at least one non-coding RNA or functional fragment thereof detected by the disclosed method is chosen from SEQ ID NO: 1 through SEQ ID NO: 1543.

In some embodiments, the sample is a human tissue sample comprising a tissue from a serum or plasma or blood draw, brushing, biopsy, or surgical resection of a subject. In some embodiments, the sample comprises total RNA from a cell that is freshly obtained, formalin fixed, alcohol-fixed and/or paraffin embedded. In some embodiments, the sample is plasma, blood or serum. In some embodiments, the sample is free of whole cells. In some embodiments, the sample comprises exosome comprising oncRNAs, but free of whole cells.

In some embodiments, the step of quantifying at least one non-coding RNA or functional fragment thereof in the sample of the disclosed method comprises isolating total RNA from the sample.

The disclosure also relates to a method of diagnosing a subject with a cancer, comprising:
a) detecting the presence, absence, and/or quantity of at least one non-coding RNA or functional fragment thereof in a sample of the subject by contacting the sample with one or a plurality of probes specific for the at least one non-coding RNA or functional fragment thereof; and
b) diagnosing the subject as having the cancer if the presence or quantity of the at least one non-coding RNA or functional fragment thereof is detected in the sample.

In some embodiments, the step of detecting is preceded by a step of acquiring the sample from the subject. In some embodiments, step a) further comprises:
i) calculating one or more scores based upon the presence, absence, or quantity of the at least one non-coding RNA or functional fragment thereof; and
ii) correlating the one or more scores to the presence, absence, or quantity of the at least one non-coding RNA or functional fragment thereof, such that, if the amount of the at least one non-coding RNA or functional fragment thereof is greater than the quantity of the at least one non-coding RNA or functional fragment thereof in a control sample; or, if the amount of the at least one non-coding RNA or functional fragment thereof is substantially equal to the quantity of the at least one non-coding RNA or functional fragment thereof in a sample taken from a subject known to have a cancer, then the subject is diagnosed as having the cancer.

In some embodiments, the one or plurality of probes used in the disclosed method comprise nucleotide sequences complementary to one or a plurality of sequences chosen from SEQ ID NO: 1 through SEQ ID NO: 1543, or one or a plurality of sequences that comprise at least about 70%, 80%, 85%, 90%, 95%, 96%, 97%, 98% or 99% sequence identity to any of nucleic acids chosen from SEQ ID NO: 1 through SEQ ID NO: 1543.

In some embodiments, the disclosed method further comprises detecting the presence, absence or quantity of an antigen of said cancer. In some embodiments, the step of quantifying the at least one non-coding RNA or functional fragment thereof in the sample of the disclosed method comprises using a fluorescence and/or digital imaging.

In some embodiments, the sample is a human tissue sample comprising a cell or tissue from a plasma, serum or blood draw, brushing, biopsy, or surgical resection of the subject. In some embodiments, the sample comprises total RNA from a cell that is freshly obtained, formalin fixed, alcohol-fixed and/or paraffin embedded. In some embodiments, the sample is human serum.

The disclosure further relates to a method of treating a subject in need thereof diagnosed with or suspected of having a cancer, comprising:
a) contacting one or a plurality of probes specific for at least one non-coding RNA or functional fragment thereof with a sample from the subject;
b) quantifying the amount of the at least one non-coding RNA or functional fragment thereof in the sample;
c) calculating one or more scores based upon the presence, absence, or quantity of the at least one non-coding RNA or functional fragment thereof;
d) correlating the one or more scores to the presence, absence, or quantity of the at least one non-coding RNA or functional fragment thereof, such that, if the amount of the at least one non-coding RNA or functional fragment thereof is greater than the quantity of the at least one non-coding RNA or functional fragment thereof in a control sample; or, if the amount of the at least one non-coding RNA or functional fragment thereof is substantially equal to the quantity of the at least one non-coding RNA or functional fragment thereof in a sample taken from a subject known to have a cancer, then the subject is diagnosed as having the cancer, and
e) administering to the subject a therapeutically effective amount of treatment for the cancer.

In some embodiments, the one or plurality of probes used in the disclosed method comprise nucleotide sequences complementary to one or a plurality of sequences chosen from SEQ ID NO: 1 through SEQ ID NO: 1543, or one or a plurality of sequences that comprise at least about 70%, 80%, 85%, 90%, 95%, 96%, 97%, 98% or 99% sequence identity to any of nucleic acids chosen from SEQ ID NO: 1 through SEQ ID NO: 1543. In some embodiments, at least one substrate comprising a fluorophore, a chemiluminescent agent, and/or a quenching agent is used in the detecting step of the disclosed method.

In some embodiments, the subject of any of the disclosed methods is a human diagnosed with or suspected as having a cancer of adrenal gland, bile ducts, bladder, blood, bone, bone marrow, brain, breast, cervix, colon, esophagus, eye, gall bladder, ganglia, gastrointestinal tract, heart, lymphatic system, liver, lung, kidney, muscle, ovary, pancreas, parathyroid, penis, prostate, prostate glands, rectum, salivary glands, skin, spine, stomach, spleen, testis, thymus, thyroid, or uterus. In some embodiments, the subject is a human diagnosed with or suspected as having a cancer chosen from: adrenal cancer, bile duct cancer, blood cancer, brain cancer, breast cancer, bone cancer, bladder cancer, cervical cancer, colon cancer, esophageal cancer, gynecologic tumors, kidney cancer, liver cancer, lung cancer, lymphocytic lymphomas, ovarian cancer, pancreatic cancer, parathyroid cancer, prostate cancer, rectal cancer, skin cancer, stomach cancer, sarcomas of soft tissues, thyroid cancer, testicular cancer, uterine cancer, cancer of the eye, cancer of the head and neck, and neoplasms of the central nervous system. In some embodiments, the subject is a human diagnosed with or suspected as having a cancer chosen from any of the cancers listed in TABLE 1. In some embodiments, the subject is a human diagnosed with or suspected as having a cancer chosen from lung cancer, breast cancer, prostate cancer, colorectal cancer, pancreas cancer, liver cancer, and ovarian cancer.

The disclosure also relates to a system comprising:
a) one or a plurality of probes and/or stains that bind to at least one non-coding RNA or functional fragment thereof; and
b) one or more devices capable of quantifying the presence, absence and/or amount of the at least one probe or stain that binds the at least one non-coding RNA and/or functional fragment thereof.

The disclosure also relates to a system comprising:
a) a sample;
b) one or a plurality of probes and/or stains that bind to at least one non-coding RNA or functional fragment thereof; and
c) one or more devices capable of quantifying the presence, absence and/or amount of the at least one probe or stain that binds the at least one non-coding RNA and/or functional fragment thereof.

In some embodiments, the one or plurality of probes comprised in the disclosed system comprise nucleotide sequences complementary to one or a plurality of sequences chosen from SEQ ID NO: 1 through SEQ ID NO: 1543, or one or a plurality of sequences that comprise at least about 70%, 80%, 85%, 90%, 95%, 96%, 97%, 98% or 99% sequence identity to any of nucleic acids chosen from SEQ ID NO: 1 through SEQ ID NO: 1543. In some embodiments, the sample is taken from a subject identified as having or suspected of having a cancer of adrenal gland, bile ducts, bladder, blood, bone, bone marrow, brain, breast, cervix, colon, esophagus, eye, gall bladder, ganglia, gastrointestinal tract, heart, lymphatic system, liver, lung, kidney, muscle, ovary, pancreas, parathyroid, penis, prostate, prostate glands, rectum, salivary glands, skin, spine, stomach, spleen, testis, thymus, thyroid, or uterus. In some embodiments, the sample is taken from a subject identified as having or suspected of having a cancer chosen from: adrenal cancer, bile duct cancer, blood cancer, brain cancer, breast cancer, bone cancer, bladder cancer, cervical cancer, colon cancer, esophageal cancer, gynecologic tumors, kidney cancer, liver cancer, lung cancer, lymphocytic lymphomas, ovarian cancer, pancreatic cancer, parathyroid cancer, prostate cancer, rectal cancer, skin cancer, stomach cancer, sarcomas of soft tissues, thyroid cancer, testicular cancer, uterine cancer, cancer of the eye, cancer of the head and neck, and neoplasms of the central nervous system. In some embodiments, the sample is taken from a subject identified as having or suspected of having a cancer chosen from any of the cancers listed in TABLE 1. In some embodiments, the subject is a human diagnosed with or suspected as having a cancer chosen from lung cancer, breast cancer, prostate cancer, colorectal cancer, pancreas cancer, liver cancer, and ovarian cancer.

The disclosure additionally relates to a method for characterizing the stage of development or pathology of a sample comprising a hyperproliferative cell, said method comprising:
a) contacting a plurality of probes specific for at least one non-coding RNA or functional fragment thereof with the sample;
b) quantifying the amount of the at least one non-coding RNA or functional fragment thereof in the sample;
c) calculating one or more normalized scores based upon the presence, absence, or quantity of the at least one non-coding RNA or functional fragment thereof; and
d) correlating the one or more scores to the quantity of the at least one non-coding RNA or functional fragment thereof, such that if the amount of the at least one non-coding RNA or functional fragment thereof is greater than the quantity of the at least one non-coding RNA or functional fragment thereof in a control sample, the correlating step comprises characterizing the sample as comprising a hyperproliferative cell.

The disclosure further relates to a method of determining whether a subject has a malignant growth, said method comprising detecting the presence, absence, or quantity of at least one non-coding RNA or functional fragment thereof in a sample of the subject by contacting the sample with:
a) a probe specific for the at least one non-coding RNA or functional fragment thereof; or
b) a substrate specific for the at least one non-coding RNA or functional fragment thereof.

In some embodiments, the one or plurality of probes used in the disclosed methods comprise nucleotide sequences complementary to one or a plurality of sequences chosen from SEQ ID NO: 1 through SEQ ID NO: 1543, or one or a plurality of sequences that comprise at least about 70%, 80%, 85%, 90%, 95%, 96%, 97%, 98% or 99% sequence identity to any of nucleic acids chosen from SEQ ID NO: 1 through SEQ ID NO: 1543. In some embodiments, the sample used in the disclosed methods is taken from a subject identified as having or suspected of having a cancer of adrenal gland, bile ducts, bladder, blood, bone, bone marrow, brain, breast, cervix, colon, esophagus, eye, gall bladder, ganglia, gastrointestinal tract, heart, lymphatic system, liver, lung, kidney, muscle, ovary, pancreas, parathyroid, penis, prostate, prostate glands, rectum, salivary glands, skin, spine, stomach, spleen, testis, thymus, thyroid, or uterus. In some embodiments, the sample used in the disclosed methods is taken from a subject identified as having or suspected of having a cancer chosen from: adrenal cancer, bile duct cancer, blood cancer, brain cancer, breast cancer, bone cancer, bladder cancer, cervical cancer, colon cancer, esophageal cancer, gynecologic tumors, kidney cancer, liver cancer, lung cancer, lymphocytic lymphomas, ovarian cancer, pancreatic cancer, parathyroid cancer, prostate cancer, rectal cancer, skin cancer, stomach cancer, sarcomas of soft tissues, thyroid cancer, testicular cancer, uterine cancer, cancer of the eye, cancer of the head and neck, and neoplasms of the central nervous system.

The disclosure relates to a method of processing RNA from a sample of a subject comprising: (i) separating small noncoding RNA in the total RNA from total mRNA; and (ii) analyzing the small noncoding RNA. In some embodiments, the method of processing further comprises obtaining a sample from a subject. In some embodiments, the method of processing further comprises the step of isolating the total RNA in the sample from other component of the sample by centrifugation. In some embodiments, the step of separating the small non-encoding RNA from the sample comprise centrifugation of the sample and removal of whole cells from the sample. In some embodiments, the step of separating the small non-encoding RNA from the sample comprises removal of exosomes from whole blood of the subject, where the sample is whole blood. In some embodiments, the analysis step comprises detecting the presence, absence or quantity of oncRNAs in the sample. In some embodiments, the oncRNA is an RNA sequence corresponding to any one or plurality of oncRNAs chosen from Table 2. In some embodiments, the analysis step comprises detecting the presence, absence or quantity of oncRNAs in the sample. In some embodiments, the oncRNA is an RNA sequence corresponding to any one or plurality of oncRNAs chosen from SEQ ID NO: SEQ ID NO: 1544 through SEQ ID NO: 6834. In some embodiments, the method of detecting the presence or absence of oncRNA in a sample comprises sequencing the RNA from a pool of isolated small, noncoding RNA in a sample. In some embodiments, the step of detecting comprises amplifying the oncRNA or cDNA complement of oncRNA from a pool of isolated, small noncoding RNA in the sample. In some embodiments, the step of detecting comprises exposing a pool of isolated, small noncoding RNA to a probe or plurality of probes specific for an RNA sequence encoded by any of cDNA sequences SEQ ID NO:1 through SEQ ID NO: 1543 or the RNA sequences of Table 2. In some embodiments, the one or plurality of probes comprise one or a plurality of the DNA sequences of Table 2 or fragments thereof. In some embodiments, the probe or plurality of probes comprises a fluorescent label. In some embodiments, the detecting comprises amplifying the RNA or cDNA sequence of oncRNA by polymerase chain reaction (PCR). In some embodiments, the probes or primers for amplifying the RNA or cDNA sequences are nucleotide sequences that are from about 4 to about 100, 200, 300, or 400 nucleotides in length. In some embodiments, the probe or primers are one or a plurality of cDNAs chosen from SEQ ID NO: 6835 through SEQ ID NO:18676 or fragment thereof that are from about 4 to about 50 nucleotides in length nested within from SEQ ID NO: 6835 through SEQ ID NO:18676.

The disclosure also relates to a composition, including, for instance a kit, tube or system, comprising or consistently essentially of one or a plurality of cDNAs chosen from SEQ ID NO: 6835 through SEQ ID NO:18676. In some embodiments, the composition comprises a cDNA that has at least about 70%, 80%, 85%, 90%, 91%, 92%, 93%, 94%, 95%, 96%, 97%, 98%, 99% sequence identity to SEQ ID N: 6835 through SEQ ID NO: 18676. In some embodiments, the composition comprises one or a plurality of cDNA molecules labeled with a fluorescent moiety.

### BRIEF DESCRIPTION OF THE DRAWINGS

FIG. 1A and FIG. 1B depict confusion matrices of the gradient boosting classifier. The TCGA data was split into training and testing sets (80/20 split), and a gradient boosting classifier was trained using the identified oncRNAs as features on the training set. A confusion matrix was computed using the classifier predictions of the test set. The confusion matrix was then normalized by row (FIG. 1A) and by column (FIG. 1B) separately for visualization purposes.

FIG. 2 depicts a bar plot of number of oncRNAs present for each cancer type. The counts represent the number of oncRNAs identified that satisfied all the filtering conditions for each cancer.

FIG. 3A, FIG. 3B and FIG. 3C depict heatmaps of oncRNAs present within breast invasive carcinoma (FIG. 3A), colon adenocarcinoma (FIG. 3B) and lung adenocarcinoma (FIG. 3C). The heatmap was generated from RNA expression data (cpm) of the oncRNAs found within each of the cancer types and plotted with all the normal samples across all tissue types. Expression data were max normalized for visualization purposes.

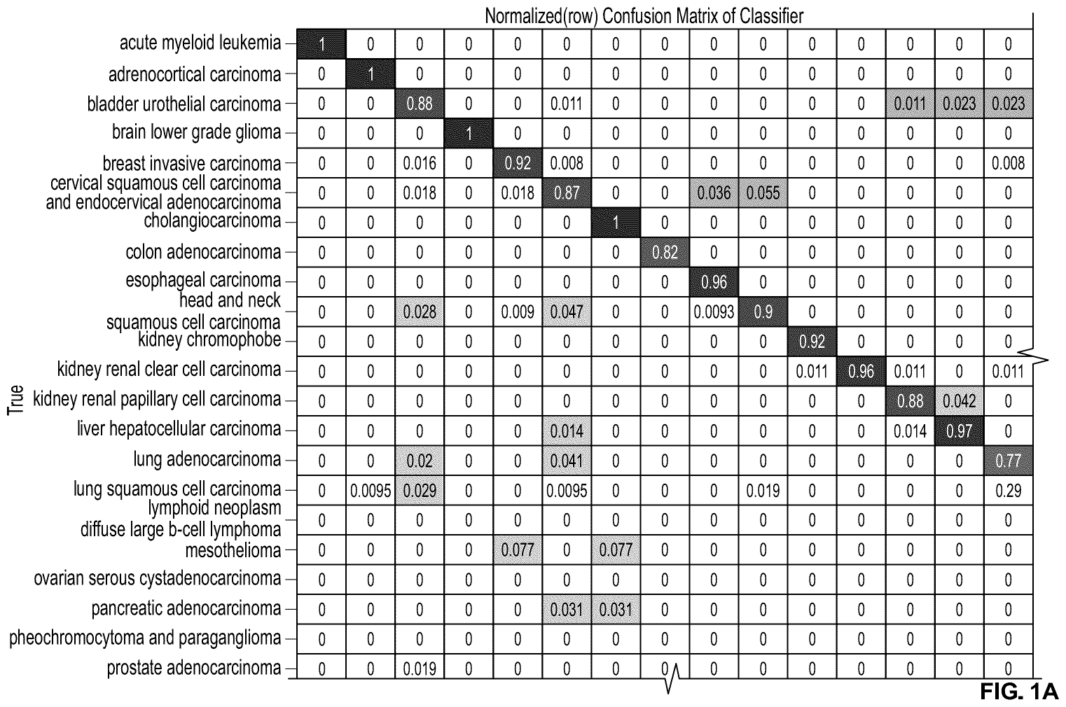

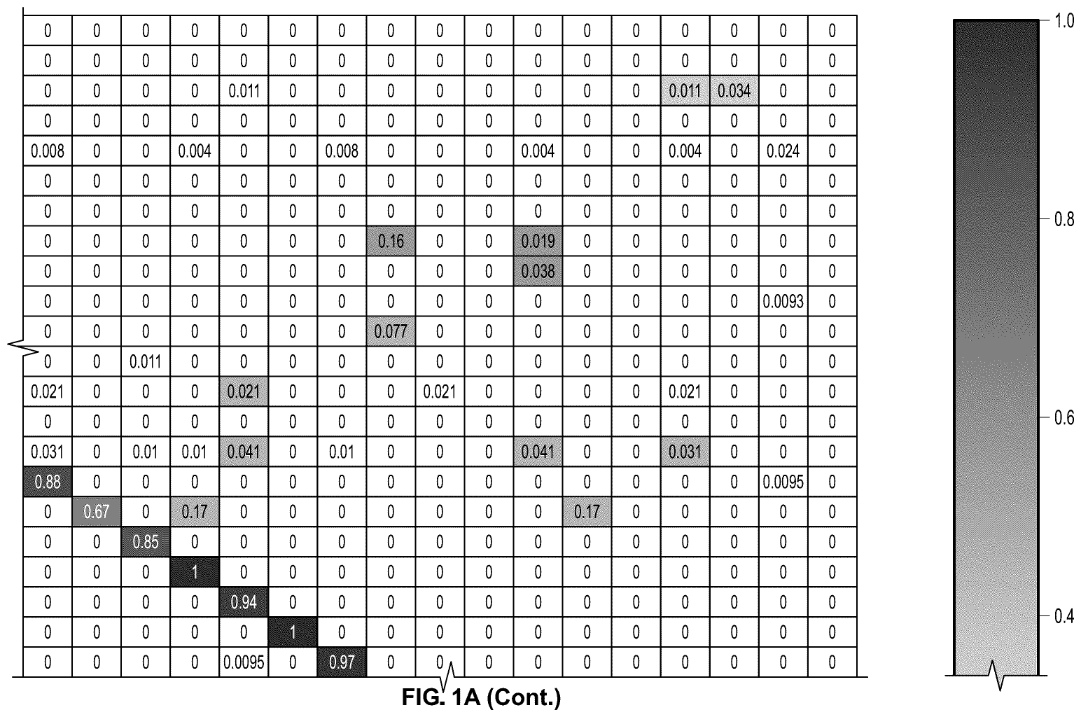

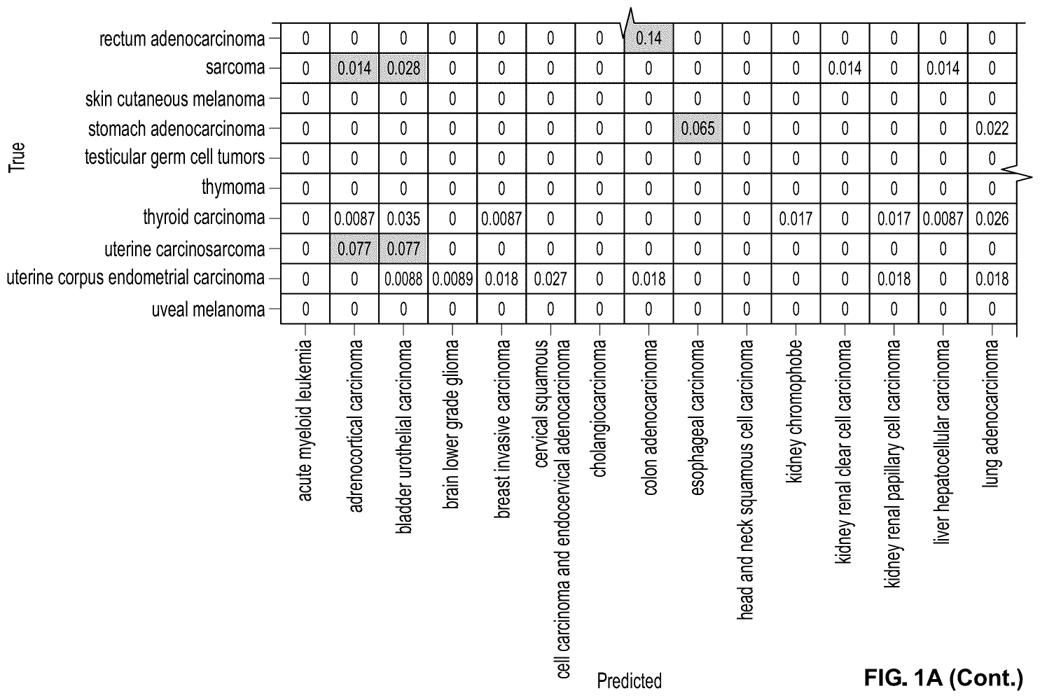

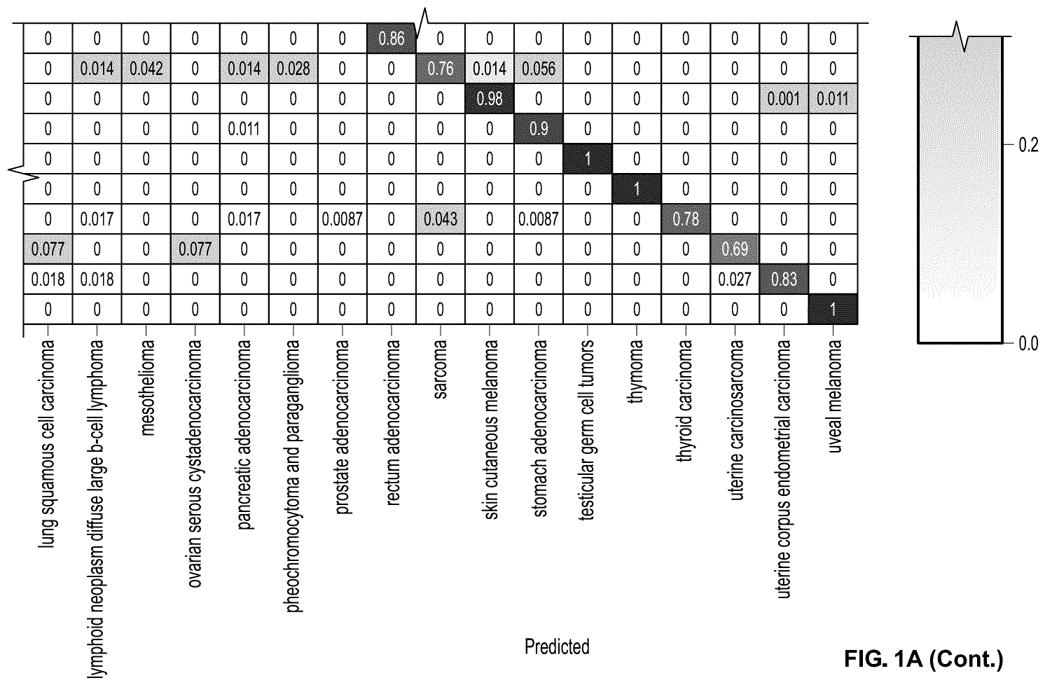

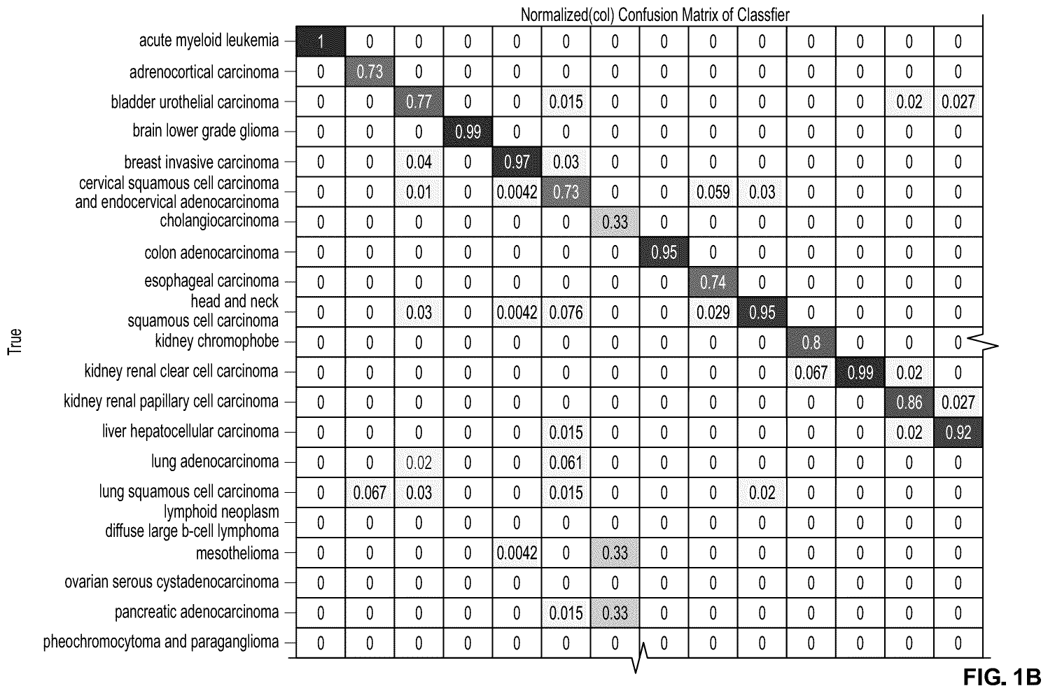

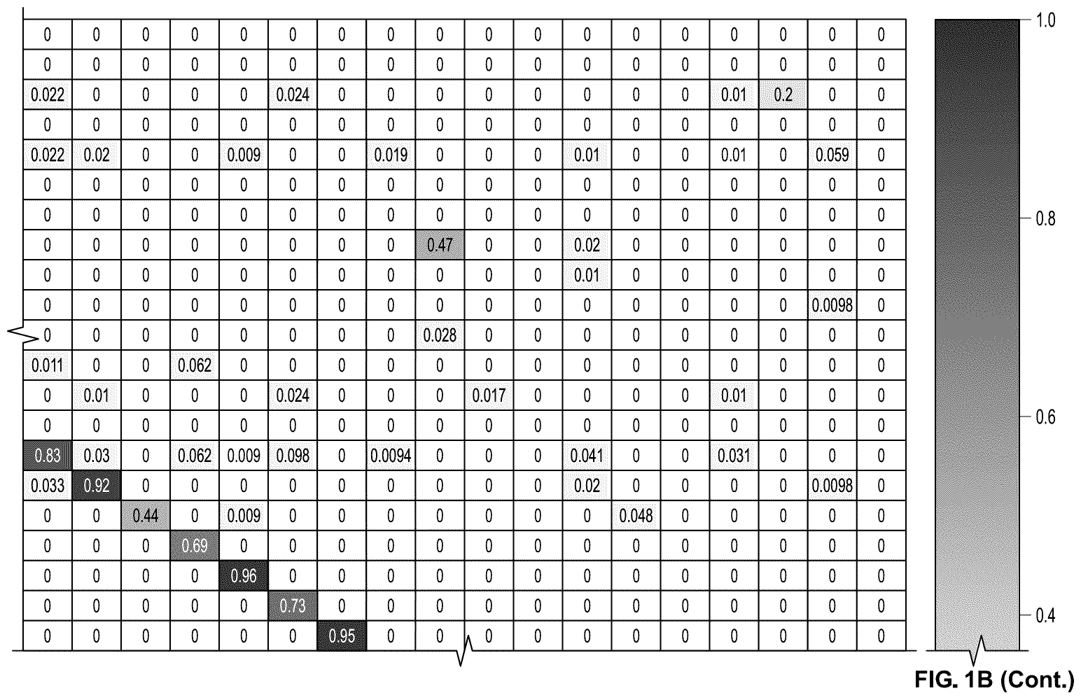

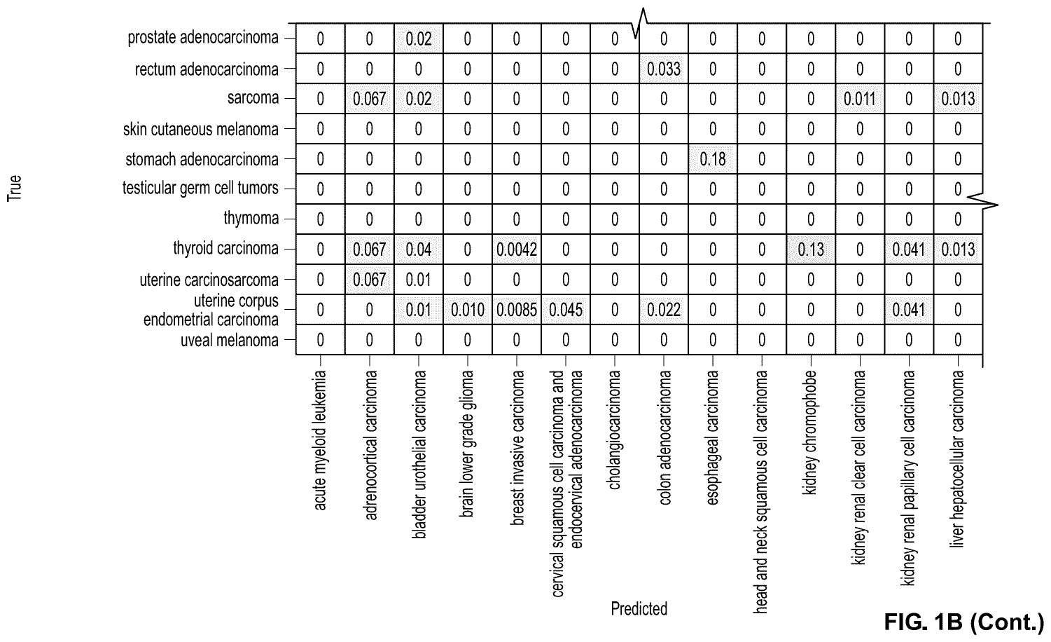

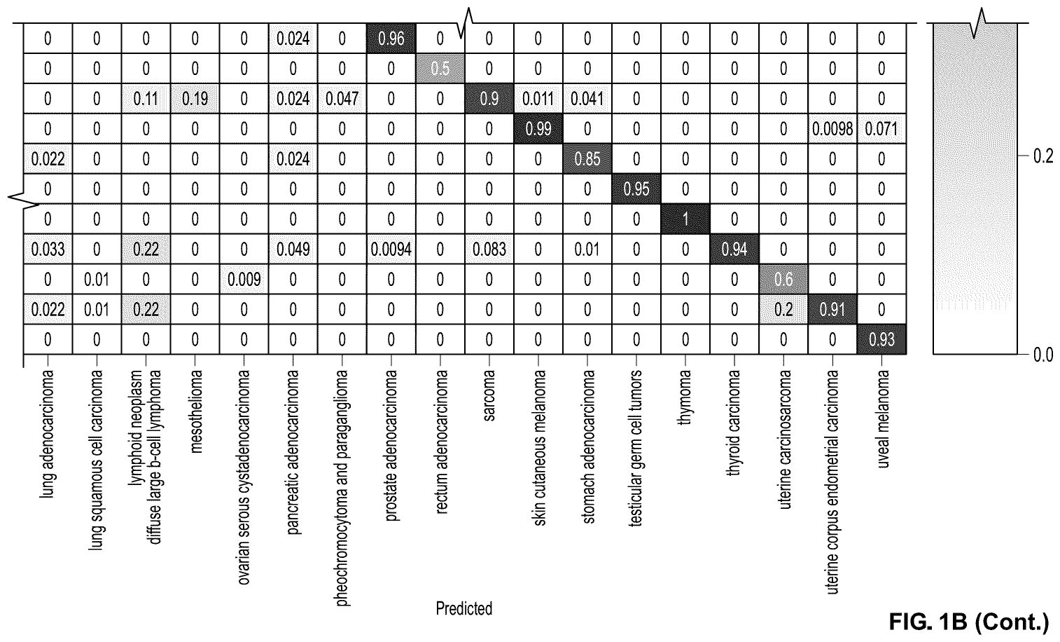

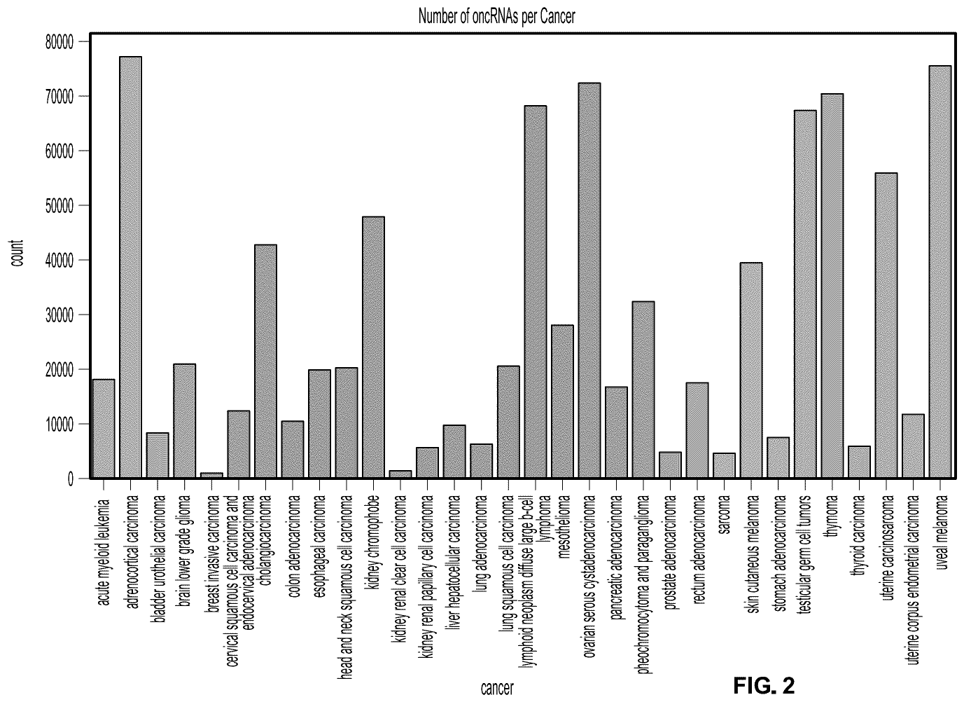

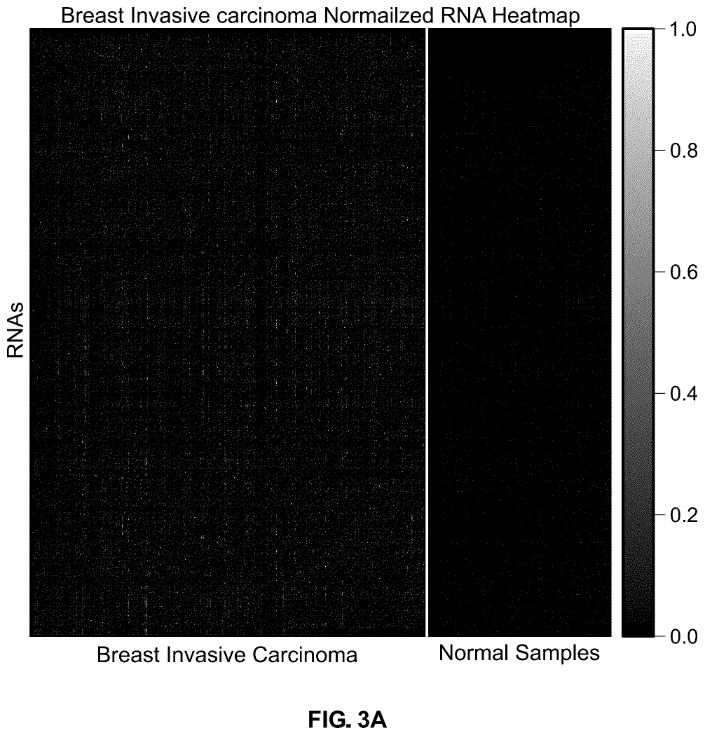

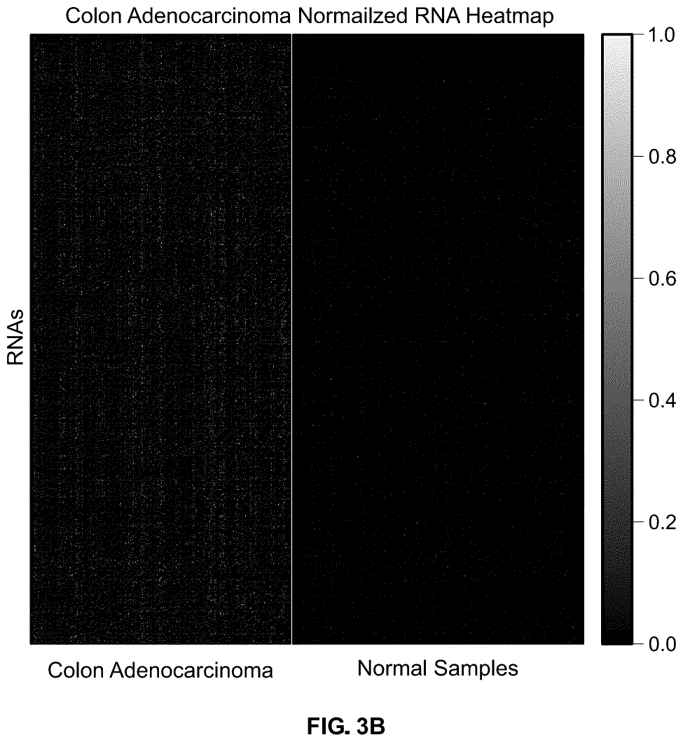

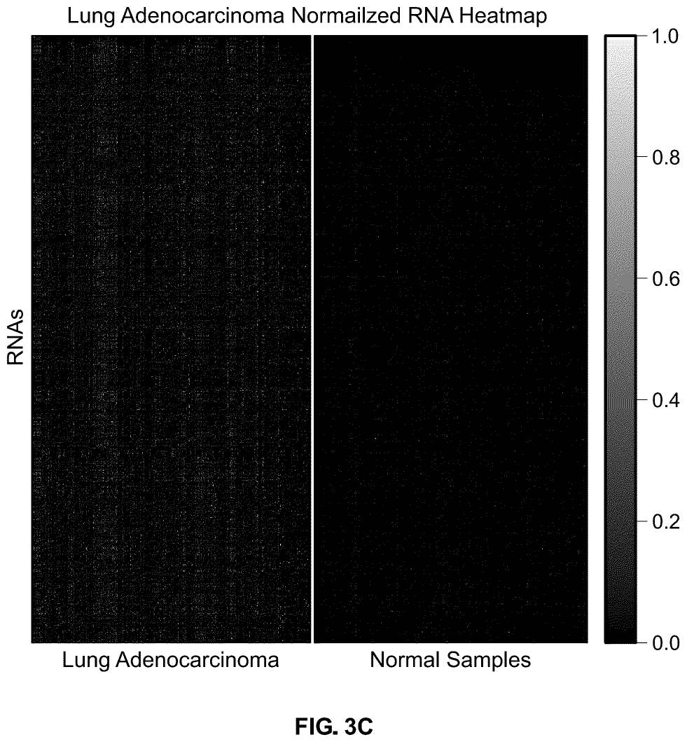

### DETAILED DESCRIPTION

The disclosure provides novel, small non-coding RNAs that serve as biomarkers which are indicative of various types of cancers, and which may be used to accurately diagnose or grade cancer in a subject. In some embodiments, the methods entail detection of extracellular, circulating small RNAs in a suitable sample.

### Definitions

Before the present methods are described, it is to be understood that the present disclosure is not limited to the particular processes, compositions, or methodologies described, as these may vary. It is also to be understood that the terminology used in the description is for the purposes of describing the particular versions or embodiments only, and is not intended to limit the scope of the present disclosure. Unless defined otherwise, all technical and scientific terms used herein have the same meanings as commonly understood by one of ordinary skill in the art. For example, Singleton et al., Dictionary of Microbiology and Molecular Biology 2nd Ed., J. Wiley & Sons (New York, NY 1994), provide one skilled in the art with a general guide to many of the terms used in the present disclosure. Moreover, the practice of the present disclosure will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual,” 2nd Ed. (Sambrook et al., 1989); “Oligonucleotide Synthesis” (M. J. Gait, Ed., 1984); “Animal Cell Culture” (R. I. Freshney, Ed., 1987); “Methods in Enzymology” (Academic Press, Inc.); “Handbook of Experimental Immunology,” 4th Ed. (D. M. Weir & C. C. Blackwell, Eds., Blackwell Science Inc., 1987); “Gene Transfer Vectors for Mammalian Cells” (J. M. Miller & M. P. Calos, Eds., 1987); “Current Protocols in Molecular Biology” (F. M. Ausubel et al., Eds., 1987); and “PCR: The Polymerase Chain Reaction,” (Mullis et al., Eds., 1994). Although any methods and materials similar or equivalent to those described herein can be used in the practice or testing of embodiments of the present disclosure, the methods, devices, and materials in some embodiments are now described. All publications mentioned herein are incorporated by reference in their entirety. Nothing herein is to be construed as an admission that the present disclosure is not entitled to antedate such disclosure by virtue of prior invention.

It must be noted that as used herein and in the appended claims, the singular forms “a,” “an,” and “the” include plural reference unless the context clearly dictates otherwise. Thus, for example, reference to a “peptide” is a reference to one or more peptides and equivalents thereof known to those skilled in the art, and so forth.

The term “about” or “approximately” is used herein to mean within the typical ranges of tolerances in the art. For example, “about” can be understood as about 2 standard deviations from the mean. According to certain embodiments, when referring to a measurable value such as an amount and the like, “about” is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value as such variations are appropriate to perform the disclosed methods. When “about” is present before a series of numbers or a range, it is understood that “about” can modify each of the numbers in the series or range.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “antibody” as used herein refers to an immunoglobulin molecule that recognizes and specifically binds a target, such as a protein, polypeptide, peptide, carbohydrate, polynucleotide, lipid, or combinations of the foregoing, through at least one antigen-binding site. As used herein, the term encompasses intact polyclonal antibodies, intact monoclonal antibodies, single chain antibodies, antibody fragments (such as Fab, Fab′, F(ab′)2, and Fv fragments), single chain Fv (scFv) antibodies, multispecific antibodies such as bispecific antibodies, monospecific antibodies, monovalent antibodies, chimeric antibodies, humanized antibodies, human antibodies, fusion proteins comprising an antigen-binding site of an antibody, and any other modified immunoglobulin molecule comprising an antigen-binding site as long as the antibodies exhibit the desired biological binding activity. An antibody can be any of the five major classes of immunoglobulins: IgA, IgD, IgE, IgG, and IgM, or subclasses (isotypes) thereof (e.g., IgG1, IgG2, IgG3, IgG4, IgA1, and IgA2), based on the identity of their heavy chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, single chain antibodies, and multispecific antibodies formed from antibody fragments. “Antibody fragment” as used herein comprises at least one antigen-binding site or epitope-binding site. The term“variable region” of an antibody refers to the variable region of an antibody light chain, or the variable region of an antibody heavy chain, either alone or in combination. The variable region of a heavy chain or a light chain generally consists of four framework regions (FR) connected by three complementarity determining regions (CDRs), also known as“hypervariable regions.” The CDRs in each chain are held together in close proximity by the framework regions and contribute to the formation of the antigen-binding site(s) of the antibody. There are at least two techniques for determining CDRs: (1) an approach based on cross-species sequence variability (i.e., Kabat et al., 1991, Sequences of Proteins of Immunological Interest, 5th Edition, National Institutes of Health, Bethesda, MD), and (2) an approach based on crystallographic studies of antigen-antibody complexes (Al-Lazikani et al., 1997, J. Mol. Biol., 273:927-948). In addition, combinations of these two approaches are sometimes used in the art to determine CDRs.

The term “biomarker” as used herein refers to a biological molecule present in an individual at varying concentrations useful in predicting the cancer status of an individual. A biomarker may include but is not limited to, nucleic acids, proteins and variants and fragments thereof. A biomarker may be DNA comprising the entire or partial nucleic acid sequence encoding the biomarker, or the complement of such a sequence. Biomarker nucleic acids useful in the invention are considered to include both DNA and RNA comprising the entire or partial sequence of any of the nucleic acid sequences of interest. In some embodiments, the biomarker is an RNA within an exosome in a sample.

The term “bodily fluid” as used herein refers to a bodily fluid including blood (or a fraction of blood such as plasma or serum), lymph, mucus, tears, saliva, sweat, sputum, urine, semen, stool, cerebrospinal fluid (CSF), breast milk, and, ascites fluid. In some embodiments, the bodily fluid is blood. In some embodiments, the bodily fluid is a fraction of blood. In some embodiments, the bodily fluid is plasma. In some embodiments, the bodily fluid is serum. In some embodiments, the bodily fluid is urine.

The terms “cancer” and “cancerous” as used herein refer to or describe a physiological condition in mammals in which a population of cells are characterized by unregulated cell growth. Thus, the term “cancer” refers to a group of diseases involving abnormal cell growth with the potential to invade or spread to other parts of the body. Examples of cancer include, but not limited to, lung cancer, bone cancer, blood cancer, chronic myelomonocytic leukemia (CMML), bile duct cancer, cervical cancer, liver cancer, pancreatic cancer, skin cancer, cancer of the head and neck, cancer of the eye, cutaneous or intraocular melanoma, uterine cancer, ovarian cancer, rectal cancer, cancer of the anal region, stomach cancer, colon cancer, breast cancer, testicular cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphomas, cancer of the bladder, cancer of the kidney or ureter (e.g., renal cell carcinoma, carcinoma of the renal pelvis), or neoplasms of the central nervous system (e.g., primary CNS lymphoma, spinal axis tumors, brain stem gliomas or pituitary adenomas).

As used herein, the term “characterizing cancer in a subject” refers to the identification of one or more properties of a cancer sample in a subject, including but not limited to, the presence of benign, pre-cancerous or cancerous tissue, the stage of the cancer, the type of the cancer, the tissue of origin of the cancer, and the subject's prognosis. Cancers may be characterized by the identification of the expression of one or more cancer marker genes, including but not limited to, the ncRNAs and/or oncRNAs disclosed herein. As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acid bases are matched according to the base pairing rules. Alternatively, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used in the specification and in the claims, the term “comprising” can include the aspects “consisting of” and “consisting essentially of.” Comprising can also mean “including but not limited to.”

The term “correlate” or “correlating” as used herein refers to a statistical association between instances of two events, where events may include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases. The present disclosure provides small non-coding RNAs, the levels of which are correlated with a particular outcome measure, such as between the level of a particular small non-coding RNA and the likelihood of developing a particular type of cancer. For example, the increased level of a small non-coding RNA may be negatively correlated with a likelihood of good clinical outcome for the patient. In this case, for example, the patient may have a decreased likelihood of long-term survival without recurrence of the cancer and/or a positive response to a chemotherapy, and the like. Such a negative correlation indicates that the patient likely has a poor prognosis or will respond poorly to a chemotherapy, and this may be demonstrated statistically in various ways, e.g., by a high hazard ratio.

As used herein, the terms “detect,” “detecting” or “detection” refer to either the general act of discovering or discerning or the specific observation of a composition. Detecting a composition may comprise determining the presence or absence of a composition. Detecting may comprise quantifying a composition. For example, detecting comprises determining the expression level of a composition. The composition may comprise a nucleic acid molecule. For example, the composition may comprise at least a portion of the ncRNAs and/or oncRNAs disclosed herein. Alternatively, or additionally, the composition may be a detectably labeled composition.

The term “diagnosed,” as used herein, refers to the recognition of a disease by the presence or detection of signs and symptoms, or genetic analysis, pathological analysis, histological analysis, and the like.

By “fragment” is meant a portion of a polypeptide or nucleic acid molecule. This portion contains, preferably, at least about 5%, 10%, 20%, 30%, 40%, 50%, 60%, 70%, 80%, 90%, 91%, 92%, 93%, 94%, 95%, 96%, 97%, 98% or about 99% of the entire length of the reference nucleic acid molecule or polypeptide. A fragment may contain about 5, 10, 20, 30, 40, 50, 60, 70, 80, 90, or 100, 200, 300, 400, 500, 600, 700, 800, 900, 1000 or more nucleotides or amino acids.

The terms “functional fragment” means any portion of a polypeptide or nucleic acid sequence from which the respective full-length polypeptide or nucleic acid relates, that portion is of a sufficient length and has a sufficient structure to confer a biological affect that is at least similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based. In some embodiments, the functional fragment is derived from the sequence of an organism, such as a human. In such embodiments, the functional fragment may retain 99%, 98%, 97%, 96%, 95%, 94%, 93%, 92%, 91%, or 90% sequence identity to the wild-type human sequence upon which the sequence is derived. In some embodiments, the functional fragment may retain 85%, 80%, 75%, 70%, 65%, or 60% sequence identity to the wild-type sequence upon which the sequence is derived.

The term “gene” refers to a nucleic acid (e.g., DNA) sequence that comprises coding sequences necessary for the production of a polypeptide, precursor, or RNA (e.g., rRNA, tRNA). The polypeptide or RNA can be encoded by a full-length coding sequence or by any portion of the coding sequence so long as the desired activity or functional properties (e.g., enzymatic activity, ligand binding, signal transduction, immunogenicity, etc.) of the full-length or fragments are retained. In some embodiments, the cells or sample of the subject comprise a hyperproliferative cell that expresses and/or secretes an oncRNA that is not present in the genome of a noncancerous cell. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

The term “hybridization” or “hybridizes” as used herein refers to the formation of a duplex between nucleotide sequences that are sufficiently complementary to form duplexes via Watson-Crick base pairing. Two nucleotide sequences are “complementary” to one another when those molecules share base pair organization homology. “Complementary” nucleotide sequences will combine with specificity to form a stable duplex under appropriate hybridization conditions. For instance, two sequences are complementary when a section of a first sequence can bind to a section of a second sequence in an anti-parallel sense wherein the 3′-end of each sequence binds to the 5′-end of the other sequence and each A, T(U), G and C of one sequence is then aligned with a T(U), A, C and G, respectively, of the other sequence. RNA sequences can also include complementary G=U or U=G base pairs. Thus, two sequences need not have perfect homology to be “complementary.” Usually two sequences are sufficiently complementary when at least about 90% (preferably at least about 95%) of the nucleotides share base pair organization over a defined length of the molecule. In the present disclosure, the capture domain of each spatial index primer comprises a region of complementarity for the nucleic acid, e.g. RNA (preferably mRNA) of the tissue sample. In some embodiments, such a region of complementarity comprised in the capture domain of each spatial index primer comprises a polythymidine sequence to capture mRNA via the poly-A tail.

The term “hyperproliferative cell” refers to a cell located in a tissue or organ having or exhibiting a “hyperproliferative disorder,” a disease or disorder characterized by abnormal proliferation, abnormal growth, abnormal senescence, abnormal quiescence, or abnormal removal of cells in an organism, and includes all forms of hyperplasias, neoplasias, and cancer. In some embodiments, a hyperproliferative cell comprises at least one or a plurality of genetic mutations that confer a co-opt or dys-regulation event of existing growth regulatory pathways within the cell. In some embodiments, the hyperproliferative cells cancer cells adopt one or a plurality of myriad strategies, such as somatic mutations, gene fusions, epigenetic modifications, and regulatory mechanism disruptions to over-activate oncogenic and to down-regulate tumor suppressive pathways. In some embodiments, the hyperproliferative disease is a cancer derived from the gastrointestinal tract or urinary system. In some embodiments, a hyperproliferative disease is a cancer of the adrenal gland, bile ducts, bladder, blood, bone, bone marrow, brain, breast, cervix, colon, esophagus, eye, gall bladder, ganglia, gastrointestinal tract, heart, lymphatic system, liver, lung, kidney, muscle, ovary, pancreas, parathyroid, penis, prostate, prostate glands, rectum, salivary glands, skin, spine, stomach, spleen, testis, thymus, thyroid, or uterus. In some embodiments, the term hyperproliferative disease is a cancer chosen from: lung cancer, bone cancer, blood cancer, chronic myelomonocytic leukemia (CMML), bile duct cancer, cervical cancer, liver cancer, pancreatic cancer, skin cancer, cancer of the head and neck, cancer of the eye, cutaneous or intraocular melanoma, uterine cancer, ovarian cancer, rectal cancer, cancer of the anal region, stomach cancer, colon cancer, breast cancer, testicular cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphomas, cancer of the bladder, cancer of the kidney or ureter (e.g., renal cell carcinoma, carcinoma of the renal pelvis), or neoplasms of the central nervous system (e.g., primary CNS lymphoma, spinal axis tumors, brain stem gliomas or pituitary adenomas).

As used herein, the phrase “in need thereof” means that the animal or mammal has been identified or suspected as having a need for the particular method or treatment. In some embodiments, the identification can be by any means of diagnosis or observation. In any of the methods and treatments described herein, the animal or mammal can be in need thereof.

The term “label” as used herein refers to any atom or molecule that can be used to provide a detectable (preferably quantifiable) effect, and that can be attached to a nucleic acid or protein. Labels include but are not limited to dyes; radiolabels such as 2P; binding moieties such as biotin; haptens such as digoxigenin; luminogenic, phosphorescent or fluorogenic moieties; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, and the like. A label may be a charged moiety (positive or negative charge) or alternatively, may be charge neutral. Labels can include or consist of nucleic acid or protein sequence, so long as the sequence comprising the label is detectable. In some embodiments, nucleic acids are detected directly without a label (e.g., directly reading a sequence).

The term “level” as used herein refers to qualitative or quantitative determination of the number of copies of a non-coding RNA transcript. An RNA transcript exhibits an “increased level” when the level of the RNA transcript is higher in a first sample, such as in a clinically relevant subpopulation of patients (e.g., patients who have cancer), than in a second sample, such as in a related subpopulation (e.g., patients who do not have cancer). In the context of an analysis of a level of an RNA transcript in a tumor sample obtained from an individual patient, an RNA transcript exhibits “increased level” when the level of the RNA transcript in the subject trends toward, or more closely approximates, the level characteristic of a clinically relevant subpopulation of patients.

The term “metastasis” as used herein refers to the process by which a cancer spreads or transfers from the site of origin to other regions of the body with the development of a similar cancerous lesion at a new location. A “metastatic” or “metastasizing” cell is one that loses adhesive contacts with neighboring cells and migrates (e.g., via the bloodstream or lymph) from the primary site of disease to secondary sites.

The term “monoclonal antibody” as used herein refers to a homogeneous antibody population involved in the highly specific recognition and binding of a single antigenic determinant or epitope. This is in contrast to polyclonal antibodies that typically include a mixture of different antibodies directed against a variety of different antigenic determinants. The term “monoclonal antibody” encompasses both intact and full-length monoclonal antibodies as well as antibody fragments (e.g., Fab, Fab′, F(ab′)2, Fv), single chain (scFv) antibodies, fusion proteins comprising an antibody portion, and any other modified immunoglobulin molecule comprising an antigen-binding site. Furthermore, “monoclonal antibody” refers to such antibodies made by any number of techniques, including but not limited to, hybridoma production, phage selection, recombinant expression, and transgenic animals.

The term “normalized” as used herein with regard to non-coding RNA transcript, refers to the level of the RNA transcript, relative to the mean levels of a set or control set of reference RNA transcripts. The reference RNA transcripts are based on their minimal variation across patients, tissues, or treatments. Alternatively, the non-coding RNA transcript may be normalized to the totality of tested RNA transcripts, or a subset of such tested RNA transcripts.

The term “oligonucleotide” as used herein refers to a short length of single-stranded polynucleotide chain. Oligonucleotides are typically less than 200 residues long (e.g., between 15 and 100); however, as used herein, the term is also intended to encompass longer polynucleotide chains. Oligonucleotides are often referred to by their length. For example, a 24 residue oligonucleotide is referred to as a “24-mer.” Oligonucleotides can form secondary and tertiary structures by self-hybridizing or by hybridizing to other polynucleotides. Such structures can include, but are not limited to, duplexes, hairpins, cruciforms, bends, and triplexes.

The word “or” as used herein means any one member of a particular list and also includes any combination of members of that list.

As used herein, the term “patient” refers to a subject afflicted with a disease or disorder. The term “patient” includes human and veterinary subjects. In some embodiments, the “patient” has been diagnosed with or suspected as having a cancer. In some embodiments, the “patient” has been diagnosed with a need for cancer treatment. In some embodiments, the “patient” has not been diagnosed with or is not suspected of having breast cancer.

A “patient response” may be assessed using any endpoint indicating a benefit to the patient, including, without limitation, (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; (5) inhibition (i.e. reduction, slowing down or complete stopping) of metastasis; (6) enhancement of anti-tumor immune response, which may, but does not have to, result in the regression or rejection of the tumor; (7) relief, to some extent, of one or more symptoms associated with the cancer, (8) increase in the length of survival following treatment; and/or (9) decreased mortality at a given point of time following treatment.

The terms “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al.). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001. Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

The terms “polynucleotide” and “nucleic acid” and “nucleic acid molecule” are used interchangeably herein and refer to polymers of nucleotides of any length, and include DNA and RNA. The polynucleotides can be deoxyribonucleotides, ribonucleotides, modified nucleotides or bases, and/or their analogs, or any substrate that can be incorporated into a polymer by DNA or RNA polymerase.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides of this disclosure may be based upon antibodies or fusion proteins, in certain embodiments, the polypeptides can occur as single chains or associated chains (e.g., dimers).

The term “prognosis” as used herein refers to the prediction of the likelihood of cancer-attributable death or progression, including recurrence, metastatic spread, and drug resistance, of neoplastic disease, such as breast cancer.

The term “reference” RNA transcript as used herein refers to an RNA transcript whose level can be used to compare the level of an RNA transcript in a test sample. In some embodiments, reference RNA transcripts include housekeeping genes, such as beta-globin, alcohol dehydrogenase, or any other RNA transcript, the level or expression of which does not vary depending on the disease status of the cell containing the RNA transcript. In another embodiments, all of the assayed RNA transcripts, or a subset thereof, may serve as reference RNA transcripts.

The term “salt” refers to acidic salts formed with inorganic and/or organic acids, as well as basic salts formed with inorganic and/or organic bases. Examples of these acids and bases are well known to those of ordinary skill in the art. Such acid addition salts will normally be pharmaceutically acceptable although salts of non-pharmaceutically acceptable acids may be of utility in the preparation and purification of the compound in question. Acid addition salts of the compounds of the disclosure are most suitably formed from pharmaceutically acceptable acids, and include for example those formed with inorganic acids e.g. hydrochloric, hydrobromic, sulphuric or phosphoric acids and organic acids e.g. succinic, maleic, acetic or fumaric acid. Other non-pharmaceutically acceptable salts e.g. oxalates can be used for example in the isolation of the compounds of the disclosure, for laboratory use, or for subsequent conversion to a pharmaceutically acceptable acid addition salt. Also included within the scope of the disclosure are solvates and hydrates. In vivo hydrolyzable esters or amides of certain compounds of the disclosure can be formed by treating those compounds having a free hydroxy or amino functionality with the acid chloride of the desired ester in the presence of a base in an inert solvent such as methylene chloride or chloroform. Suitable bases include triethylamine or pyridine. Conversely, compounds of the disclosure having a free carboxy group can be esterified using standard conditions which can include activation followed by treatment with the desired alcohol in the presence of a suitable base. Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconite derived from aconitic acid, the ascorbate derived from ascorbic acid, the benzenesulphonate derived from benzenesulphonic acid, the benzoate derived from benzoic acid, the cinnamate derived from cinnamic acid, the citrate derived from citric acid, the embonate derived from embonic acid, the enanthate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. In some embodiments, the salts are sodium, lysine and arginine salts of the compounds of the disclosure. Such salts can be formed by procedures well known and described in the art.

Other acids such as oxalic acid, which cannot be considered pharmaceutically acceptable, can be useful in the preparation of salts useful as intermediates in obtaining a chemical compound of the disclosure and its pharmaceutically acceptable acid addition salt. Metal salts of a chemical compound of the disclosure include alkali metal salts, such as the sodium salt of a chemical compound of the disclosure containing a carboxy group. Mixtures of isomers obtainable according to the disclosure can be separated in a manner known per se into the individual isomers; diastereoisomers can be separated, for example, by partitioning between polyphasic solvent mixtures, recrystallization and/or chromatographic separation, for example over silica gel or by, e.g., medium pressure liquid chromatography over a reversed phase column, and racemates can be separated, for example, by the formation of salts with optically pure salt-forming reagents and separation of the mixture of diastereoisomers so obtainable, for example by means of fractional crystallization, or by chromatography over optically active column materials.

As used herein, the term “sample” refers to a biological sample obtained or derived from a source of interest, as described herein. In some embodiments, a source of interest comprises an organism, such as an animal or human. In some embodiments, a biological sample comprises biological tissue or fluid. In some embodiments, a biological sample may be or comprise bone marrow, blood, blood cells, ascites, tissue or fine needle biopsy samples, cell-containing body fluids, free floating nucleic acids, sputum, saliva, urine, cerebrospinal fluid, peritoneal fluid, pleural fluid, feces, lymph, gynecological fluids, skin swabs, vaginal swabs, oral swabs, nasal swabs, washings or lavages such as a ductal lavages or broncheoalveolar lavages, aspirates, scrapings, bone marrow specimens, tissue biopsy specimens, surgical specimens, feces, other body fluids, secretions and/or excretions, and/or cells therefrom, etc. In some embodiments, a biological sample is or comprises cells obtained from an individual. In some embodiments, the sample comprises an exosome or oncRNA sequences disclosed herein (or fragments thereof—such as nucleic acid sequences that have from about 75% to about 99% sequence identity to nucleic acid sequences identified in TABLE 1). In some embodiments, the sample comprises an exosome or oncRNA sequences within an exosome or plurality of exosomes, optionally in serum or blood, wherein the sequences comprise one or a plurality of sequences chosen from TABLE 2. the sample comprises an exosome or oncRNA sequences within an exosome or plurality of exosomes, optionally in serum or blood, wherein the sequence comprise one or a plurality of sequences that are fragments have from about 75% to about 99% sequence identity to nucleic acid sequences identified in TABLE 2. In some embodiments, the oncRNA sequences are disassociated from a whole cell. In some embodiments, the sample comprises an exosome and/or nucleic acid sequences disassociated from a whole cell and the sample is free of whole cells. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc. In some embodiments, the “sample” is a sample containing circulating small non-coding RNAs. In some embodiments, the sample is a processed sample that has been passed through a filter, or is centrifuged and resuspended to remove whole cells.

A “score” is a numerical value that may be assigned or generated after normalization of the value based upon the presence, absence, or quantity of one or a plurality of non-coding RNAs in a subject. In some embodiments, the score is normalized in respect to a control data value. In some embodiments, the control dataset is a negative control dataset.

The term “small non-coding RNA” or “small ncRNA” (sRNA) as used herein, refers to RNA that is not translated into protein and includes transfer RNA (tRNA), ribosomal RNA (rRNA), small nucleolar RNA (snoRNA), microRNA (miRNA), small interfering RNA (siRNA), small nuclear (snRNA), Y RNA, vault RNA, antisense RNA, transcription initiation RNA (tiRNA), transcriptional start-site associated RNA (TSSa-RNA) and piwi interacting RNA (piRNA). Small ncRNAs are generally 18 to 200 nucleotides in length. In some embodiments, a small ncRNA as used herein is between 50 and 100 nucleotides. A ncRNA may be of endogenous origin (e.g., a human small non-coding RNA) or exogenous origin (e.g., virus, bacteria, parasite). “Canonical” ncRNA refers to the sequence of the RNA as predicted from the genome sequence and is the most abundant sequence identified for a particular RNA. “Trimmed” ncRNA refers to an ncRNA in which exonuclease-mediated nucleotide trimming has removed one or more nucleotides at the 5′ and/or 3′ end of the molecule. “Extended ncRNA” refers to an small non-coding RNA that is longer than the canonical small non-coding RNA sequence and is a term recognized in the art. “Orphan ncRNA” or “oncRNA” refers to a small ncRNA that is cancer-specific with unknown function. The nucleotides making up the extension correspond to nucleotides of the precursor sequence and are therefore encoded by the genome in contrast to non-templated nucleotide addition. In some embodiments, any of the methods disclosed herein comprise detecting any one or a combination of small ncRNAs disclosed above.

The term “stringency” is used in reference to the conditions of temperature, ionic strength, and the presence of other compounds such as organic solvents, under which nucleic acid hybridizations are conducted. Under “low stringency conditions,” a nucleic acid sequence of interest will hybridize to its exact complement, sequences with single base mismatches, closely related sequences (e.g., sequences with 90% or greater identity), and sequences having only partial identity (e.g., sequences with 50-90% identity). Under “medium stringency conditions,” a nucleic acid sequence of interest will hybridize only to its exact complement, sequences with single base mismatches, and closely relation sequences (e.g., 90% or greater identity). Under “high stringency conditions,” a nucleic acid sequence of interest will hybridize only to its exact complement, and (depending on conditions such a temperature) sequences with single base mismatches. In other words, under conditions of high stringency, the temperature can be raised so as to exclude hybridization to sequences with single base mismatches. Thus, the term “high stringency” as used herein refers to conditions that: (1) employ low ionic strength and high temperature for washing, for example 15 mM sodium chloride/1.5 mM sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 in 5×SSC (0.75M NaCl, 75 mM sodium citrate) at 42° C.; or (3) employ during hybridization 50% formamide in 5×SSC, 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC and 50% formamide, followed by a wash consisting of 0.1×SSC containing EDTA at 55° C.

The term “subject” used herein refers to a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child and adolescent, whether male or female, are intended to be covered. In some embodiments, the subject is any organisms that are screened using the diagnostic methods described herein. In some embodiments, the subject is a human diagnosed with or suspected as having a cancer. In some embodiments, the subject is a human in need of treatment for an underlying disease or disorder, such as but not limited to cancer.

By “substantially identical” is meant a nucleic acid molecule (or polypeptide) comprises at least about 50% sequence identity to a reference nucleic acid sequence (for example, any one of the nucleic acid sequences described herein) or amino acid sequence. In some embodiments, such a sequence is at least about 60%, 70%, 80%, 85%, 90%, 95% or even 99% identical at the nucleic acid level or amino acid level to the reference sequence used for comparison.

The term “therapeutically effective amount” means a quantity sufficient to achieve a desired therapeutic effect, for example, an amount which results in the prevention or amelioration of or a decrease in the symptoms associated with a disease that is being treated, e.g., disorders associated with cancer growth or a hyperproliferative disorder. The amount of compound administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The regimen of administration can affect what constitutes an effective amount. Further, several divided dosages, as well as staggered dosages, can be administered daily or sequentially, or the dose can be continuously infused, or can be a bolus injection. Further, the dosages of the compound(s) of the disclosure can be proportionally increased or decreased as indicated by the exigencies of the therapeutic or prophylactic situation. Typically, an effective amount of the compounds of the present disclosure, sufficient for achieving a therapeutic effect, range from about 0.000001 mg per kilogram body weight per day to about 10,000 mg per kilogram body weight per day. Preferably, the dosage ranges are from about 0.0001 mg per kilogram body weight per day to about 100 mg per kilogram body weight per day. The compounds disclosed herein can also be administered in combination with each other, or with one or more additional therapeutic compounds.

The terms “treatment” or “treating” as used herein is an approach for obtaining beneficial or desired results including clinical results for the subject. For purposes herein, beneficial or desired clinical results include, but are not limited to, one or more of the following: (1) preventing or delaying the appearance of clinical symptoms of the state, disorder, or condition developing in a person who may be afflicted with or predisposed to the state, disorder or condition but does not yet experience or display clinical symptoms of the state, disorder or condition; (2) inhibiting the state, disorder or condition, i.e., arresting, reducing or delaying the development of the disease or a relapse thereof (in case of maintenance treatment) or at least one clinical symptom, sign, or test, thereof; or (3) relieving the disease, i.e., causing regression of the state, disorder or condition or at least one of its clinical or sub-clinical symptoms or signs. In some embodiments, a subject is successfully “treated” according to the methods of the present disclosure if the patient shows one or more of the following: a reduction in the number of and/or complete absence of cancer cells; a reduction in the tumor size; an inhibition of tumor growth; inhibition of and/or an absence of cancer cell infiltration into peripheral organs including the spread of cancer cells into soft tissue and bone; inhibition of and/or an absence of tumor or cancer cell metastasis; inhibition and/or an absence of cancer growth; relief of one or more symptoms associated with the specific cancer; reduced morbidity and mortality; improvement in quality of life; reduction in tumorigenicity; reduction in the number or frequency of cancer stem cells; or some combination of such effects.

The term “tumor” as used herein, refers to all neoplastic cell growth and proliferation, whether malignant or benign, and all pre-cancerous and cancerous cells and tissues. A “benign” tumor is not cancerous and it does not invade nearby tissue or spread to other parts of the body. A “premalignant” tumor is a tumor which is not yet cancerous but has the potential to become malignant. A “malignant” tumor, on the other hand, is cancerous and can grow and spread to other parts of the body.

The term “tumor sample” as used herein refers to a sample comprising tumor material obtained from a cancer patient. The term encompasses tumor tissue samples, for example, tissue obtained by surgical resection and tissue obtained by biopsy, such as for example, a core biopsy or a fine needle biopsy. In some embodiments, the tumor sample is a fixed, wax-embedded tissue sample, such as a formalin-fixed, paraffin-embedded tissue sample. Additionally, the term “tumor sample” encompasses a sample comprising tumor cells obtained from sites other than the primary tumor, e.g., circulating tumor cells. The term also encompasses cells that are the progeny of the patient's tumor cells, e.g. cell culture samples derived from primary tumor cells or circulating tumor cells. The term further encompasses samples that may comprise protein or nucleic acid material shed from tumor cells in vivo, e.g., bone marrow, blood, plasma, serum, and the like. The term also encompasses samples that have been enriched for tumor cells or otherwise manipulated after their procurement and samples comprising polynucleotides and/or polypeptides that are obtained from a patient's tumor material.

### Small Non-Coding RNA as Biomarkers for Cancer

The human genome encodes for a vast amount of non-coding RNA (ncRNA) transcripts. Non-coding RNAs can be grouped into two classes based on transcript size: small ncRNAs (smRNAs) and long ncRNAs (lncRNAs). Small ncRNAs are generally 18 to 200 nucleotides in length while lncRNAs are greater than 200 nucleotides. Small ncRNAs have diverse cellular functions and consist of several classes including the highly abundant transfer RNAs (tRNAs), ribosomal RNAs (rRNAs), small nucleolar RNAs (snoRNAs), microRNAs (miRNAs), small interfering RNAs (siRNAs), small nuclear RNAs (snRNAs), and piwi-interacting RNAs (piRNAs) (Amaral et al., 2008; Martens-Uzunova et al., 2013). Small non-coding RNAs act as translational repressors by binding to target mRNAs at sites with adequate sequence complementary (Ameres et al., 2007), while the highly abundant cytoplasmic Y RNAs function in RNA quality control by affecting the subcellular location of Ro proteins (Sim et al., 2009). The repressive activity of mature small non-coding RNAs on mRNA translation is shared by other classes of ncRNAs, including siRNAs and endo-siRNAs, in addition to piRNAs that silence retrotransposons at defined subcellular locations (Chuma and Pillai, 2009). Small non-coding RNA activity relies on sufficient levels of abundance in the cytoplasm, and interaction with RNA-induced silencing complexes (RISC) localized at endosomal membranes (Gibbings et al., 2009; Lee et al., 2009a), whereas low abundant small non-coding RNAs have less impact on translational repression. As a consequence, subtle alterations in the levels of certain small non-coding RNA may already influence cellular processes, while strong perturbations can cause disease. Besides abundance, interactions with (RISC) proteins but also RNA partners and correct subcellular localization are interrelated factors that control small non-coding RNA physiology (Mullokandov et al., 2012; Wee et al., 2012).

Small RNAs can be secreted in cell-derived extracellular vesicles, such as exosomes. Both mRNA and small non-coding RNA species have been found contained in exosomes. As such, exosomes can provide a means for transfer and protection of RNA content from degradation in the environment, enabling a stable source for reliable detection of RNA biomarkers.

The disclosure relates to small non-coding RNA biomarkers found to be differentially present in biological samples derived from subjects having cancer, as compared with subjects who are “normal,” i.e., subjects who do not have cancer. A small non-coding RNA biomarker or set of small non-coding RNA biomarkers is differentially present between samples if the difference between the levels of expression of the small non-coding RNA biomarker or set of small non-coding RNA biomarkers in the samples is determined to be statistically significant. Common tests for statistical significance include, but are not limited to, t-test, ANOVA, Kniskal-Wallis, Wilcoxon, Mann-Whitney, and odds ratio. Small non-coding RNA biomarkers, alone or in combination, can be used to provide a measure of the relative risk that a subject has or does not have cancer.

Small non-coding RNA biomarkers of various types of cancer were discovered by small RNA sequencing of multiple types of cancer from various tissues of origin, and identifying previously unknown small non-coding RNAs that are specifically expressed in the cancer cells. Hundreds of previously unknown small non-coding RNAs that are specifically expressed in the cancer cells, particularly cancer cells from lung, breast, prostate, colorectal, pancreas, liver and ovarian, were identified in this manner and disclosed in TABLE 1 (SEQ ID NO: 1-1524) with their respective cancerous indications provided. These small non-coding RNAs can now be used as biomarkers to determine the type of cancer and the status thereof of a subject, for example, a subject whose cancer status was previously unknown or who is suspected to be suffering from cancer. This may be accomplished by determining the level of one or more of the small non-coding RNAs disclosed herein, or combinations thereof, in a biological sample of the subject. A difference in the level of one or more of these small non-coding RNA biomarkers as compared to that in a biological sample of a normal or healthy subject is an indication that the subject has cancer of the type and tissue of origin associated with the one or more of the small non-coding RNAs detected in the sample of the subject, which may be early-stage, moderate or mid-stage, or severe or late-stage of cancer.

In some embodiments, the level of one or more disclosed small non-coding RNA biomarkers may be used to detect and/or diagnose cancer in a subject suspected of having a cancer but otherwise having no symptoms associated with the cancer. In some embodiments, the level of one or more disclosed small non-coding RNA biomarkers may be used to detect and/or diagnose cancer in a subject having symptoms characteristic of early-stage cancer.

In some embodiments, the level of one or more disclosed small non-coding RNA biomarkers may be used to monitor the course of cancer progression in a subject. The cancer status of a subject can change over time. For example, the cancer may worsen or improve over time during the course of a treatment regime. With such worsening or improvement, the level of one or more small non-coding RNA biomarkers may change in a statistically significant fashion, as detected in samples of the subject. For example, the level of one or more disclosed small non-coding RNA biomarker may increase over time with the development of the cancer. Thus, the course of cancer progression in a subject can be monitored by determining the level of one or more disclosed small non-coding RNA biomarkers in a first sample obtained from the subject, and determining the level of the same one or more small non-coding RNA biomarkers in a second sample obtained from the subject, where the second sample is obtained after the first sample. The levels in the second sample relative to the levels in the first sample are indicative of disease progression. For example, an increase in the level of one or more disclosed small non-coding RNA biomarkers from TABLE 1 from the first sample as compared to the second sample is indicative that the subject has developed the type of cancer associated with the small non-coding RNA biomarkers used for detection as provided in TABLE 1, or that the disease has worsened. Conversely, a decrease in the level of one or more disclosed small non-coding RNA biomarkers from TABLE 1 from the first sample as compared to the second sample indicates that the disease has improved.

Whether or not the level of a small non-coding RNA biomarker in a biological sample obtained from a test subject is different from the level of the small non-coding RNA biomarker present in a normal subject may be ascertained by comparing the level of the small non-coding RNA biomarker in the sample from the test subject with a suitable control. The skilled person can select an appropriate control for the assay in question. For example, a suitable control may be a biological sample from a subject known to be healthy and does not have cancer (e.g., a negative control). If a suitable control is obtained from a normal subject, a statistically significant difference in the level of a small non-coding RNA biomarker in a test subject relative to the suitable control is indicative that the subject has the type of cancer associated with the particular small non-coding RNA biomarker detected. In some embodiments, the difference in the level of a small non-coding RNA biomarker is an increase in the test subject as compared to the control. A suitable control may also be a reference standard. A reference standard serves as a reference level for comparison, such that test samples can be compared to the reference standard in order to infer the type of cancer, tissue of origin, and status of the cancer in the test subject. A reference standard may be representative of the level of one or more disclosed small non-coding RNA biomarkers in a known subject, e.g., a subject known to be a normal subject, or a subject known to have cancer. Likewise, a reference standard may be representative of the level of one or more disclosed small non-coding RNA biomarkers in a population of known subjects, e.g., a population of subjects known to be normal subjects, or a population of subjects known to have a particular type of cancer. The reference standard may be obtained, for example, by pooling samples from a plurality of individuals and determining the level of a particular small non-coding RNA biomarker in the pooled samples, thereby producing a standard over an averaged population. Such a reference standard represents an average level of the particular small non-coding RNA biomarker among a population of individuals. A reference standard may also be obtained, for example, by averaging the level of a particular small non-coding RNA biomarker determined to be present in individual samples obtained from a plurality of individuals. Such a standard is also representative of an average level of the particular small non-coding RNA biomarker among a population of individuals. A reference standard may also be a collection of values each representing the level of a particular small non-coding RNA biomarker in a known subject in a population of individuals. In some embodiments, test samples may be compared against such a collection of values in order to infer the type of cancer, tissue of origin, and the status or stage of the cancer in a subject. In some embodiments, the reference standard is an absolute value. In such embodiments, test samples may be compared against the absolute value in order to infer the type of cancer, tissue of origin, and the status or stage of the cancer in a subject. In some embodiments, a comparison between the level of one or more disclosed small non-coding RNA biomarkers in a sample relative to a suitable control is made by executing a software classification algorithm.

In some embodiments, the increased expression of one or more disclosed small non-coding RNAs is an increase of about 10% or more expression than the expression of the same non-coding RNAs in a normal sample. In some embodiments, the increased expression of one or more disclosed small non-coding RNAs is an increase of about 20% or more expression than the expression of the same non-coding RNAs in a normal sample. In some embodiments, the increased expression of one or more disclosed small non-coding RNAs is an increase of about 30% or more expression than the expression of the same non-coding RNAs in a normal sample. In some embodiments, the increased expression of one or more disclosed small non-coding RNAs is an increase of about 40% or more expression than the expression of the same non-coding RNAs in a normal sample. In some embodiments, the increased expression of one or more disclosed small non-coding RNAs is an increase of about 50% or more expression than the expression of the same non-coding RNAs in a normal sample. In some embodiments, the increased expression of one or more disclosed small non-coding RNAs is an increase of about 60% or more expression than the expression of the same non-coding RNAs in a normal sample. In some embodiments, the increased expression of one or more disclosed small non-coding RNAs is an increase of about 70% or more expression than the expression of the same non-coding RNAs in a normal sample. In some embodiments, the increased expression of one or more disclosed small non-coding RNAs is an increase of about 80% or more expression than the expression of the same non-coding RNAs in a normal sample. In some embodiments, the increased expression of one or more disclosed small non-coding RNAs is an increase of about 90% or more expression than the expression of the same non-coding RNAs in a normal sample. In some embodiments, the increased expression of one or more disclosed small non-coding RNAs is an increase of about 100% or more expression than the expression of the same non-coding RNAs in a normal sample.

In some embodiments, the increased expression of one or more disclosed non-coding RNAs is an increase of about 2-fold or more expression than the expression of the same non-coding RNAs in a normal sample. In some embodiments, the increased expression of one or more disclosed non-coding RNAs is an increase of about 3-fold or more expression than the expression of the same non-coding RNAs in a normal sample. In some embodiments, the increased expression of one or more disclosed non-coding RNAs is an increase of about 4-fold or more expression than the expression of the same non-coding RNAs in a normal sample. In some embodiments, the increased expression of one or more disclosed non-coding RNAs is an increase of about 5-fold or more expression than the expression of the same non-coding RNAs in a normal sample. In some embodiments, the increased expression of one or more disclosed non-coding RNAs is an increase of about 6-fold or more expression than the expression of the same non-coding RNAs in a normal sample. In some embodiments, the increased expression of one or more disclosed non-coding RNAs is an increase of about 7-fold or more expression than the expression of the same non-coding RNAs in a normal sample. In some embodiments, the increased expression of one or more disclosed non-coding RNAs is an increase of about 8-fold or more expression than the expression of the same non-coding RNAs in a normal sample. In some embodiments, the increased expression of one or more disclosed non-coding RNAs is an increase of about 9-fold or more expression than the expression of the same non-coding RNAs in a normal sample. In some embodiments, the increased expression of one or more disclosed non-coding RNAs is an increase of about 10-fold or more expression than the expression of the same non-coding RNAs in a normal sample.

In some embodiments, the one or more non-coding RNAs detected in a sample comprise at least about 70%, 80%, 81%, 82%, 83%, 84, 85%, 86%, 87%, 88%, 89%, 90%, 91%, 92%, 93%, 94%, 95%, 96%, 97%, 98%, 99% or 100% sequence identity to any of the nucleic acid sequences disclosed in TABLE 1 (SEQ ID NO: 1 through SEQ ID NO: 1543). In some embodiments, the one or more non-coding RNAs detected in a sample comprise at least about 70%, 80%, 81%, 82%, 83%, 84, 85%, 86%, 87%, 88%, 89%, 90%, 91%, 92%, 93%, 94%, 95%, 96%, 97%, 98%, 99% or 100% sequence identity to any of the nucleic acid sequences disclosed in TABLE 2 (SEQ ID NO: 1544 through SEQ ID NO: 6834). In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 2. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 3. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 4. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 5. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 6. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 7. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 8. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 9. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 10. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 11. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 12. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 13. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 14. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 15. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 16. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 17. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 18. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 19. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 20. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 21. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 22. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 23. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 24. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 25. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 26. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 27. In some embodiments, the one or more non-coding RNAs detected in a sample comprise at least 70%, 75%, 80%, 85%, 90%, 91%, 92%, 93%, 94%, 95%, 96%, 97%, 98%, 99% or 100% sequence identity to SEQ ID NO: 28. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 29. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 30. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 31. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 32. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 33. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 34. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 35. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 36. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 37. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 38. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 39. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 40. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 41. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 42. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 43. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 44. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 45. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 46. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 47. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 48. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 49. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 50. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 51. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 52. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 53. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 54. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 55. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 56. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 57. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 58. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 59. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 60. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 61. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 62. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 63. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 64. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 65. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 66. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 67. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 68. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 69. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 70. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 71. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 72. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 73. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 74. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 75. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 76. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 77. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 78. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 79. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 80. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 81. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 82. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 83. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 84. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 85. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 86. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 87. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 88. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 89. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 90. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 91. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 92. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 93. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 94. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 95. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 96. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 97. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 98. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 99. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 100. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 101. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 102. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 103. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 104. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 105. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 106. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 107. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 108. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 109. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 110. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 111. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 112. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 113. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 114. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 115. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 116. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 117. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 118. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 119. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 120. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 121. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 122. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 123. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 124. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 125. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 126. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 127. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 128. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 129. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 130. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO:131. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 132. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 133. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 134. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 135. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 136. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 137. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 138. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 139. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 140. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 141. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 142. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 143. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 144. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 145. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 146. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 147. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 148. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 149. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 150. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 151. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 152. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 153. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 154. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 155. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 156. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 157. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 158. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 159. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 160. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 161. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 162. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 163. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 164. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 165. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 166. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 167. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 168. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 169. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 170. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 171. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 172. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 173. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 174. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 175. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 176. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 177. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 178. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 179. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 180. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 181. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 182. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 183. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 184. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 185. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 186. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 187. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 188. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 189. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 190. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 191. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 192. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 193. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 194. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 195. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 196. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 197. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 198. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 199. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 200. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 201. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 202. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 203. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 204. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 205. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 206. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 207. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 208. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 209. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 210. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 211. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 212. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 213. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 214. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 215. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 216. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 217. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 218. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 219. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 220. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 221. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 222. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 223. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 224. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 225. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 226. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 227. In some embodiments, the one or more non-coding RNAs detected in a sample comprise at least 70%, 75%, 80%, 85%, 90%, 91%, 92%, 93%, 94%, 95%, 96%, 97%, 98%, 99% or 100% sequence identity to SEQ ID NO: 228. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 229. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 230. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 231. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 232. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 233. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 234. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 235. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 236. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 237. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 238. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 239. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 240. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 241. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 242. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 243. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 244. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 245. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 246. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 247. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 248. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 249. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 250. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 251. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 252. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 253. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 254. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 255. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 256. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 257. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 258. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 259. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 260. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 261. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 262. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 263. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 264. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 265. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 266. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 267. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 268. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 269. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 270. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 271. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 272. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 273. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 274. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 275. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 276. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 277. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 278. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 279. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 280. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 281. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 282. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 283. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 284. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 285. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 286. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 287. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 288. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 289. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 290. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 291. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 292. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 293. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 294. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 295. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 296. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 297. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 298. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 299. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 300. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 301. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 302. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 303. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 304. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 305. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 306. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 307. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 308. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 309. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 310. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 311. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 312. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 313. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 314. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 315. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 316. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 317. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 318. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 319. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 320. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 321. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 322. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 323. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 324. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 325. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 326. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 327. In some embodiments, the one or more non-coding RNAs detected in a sample comprise at least 70%, 75%, 80%, 85%, 90%, 91%, 92%, 93%, 94%, 95%, 96%, 97%, 98%, 99% or 100% sequence identity to SEQ ID NO: 328. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 329. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 330. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 331. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 332. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 333. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 334. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 335. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 336. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 337. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 338. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 339. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 340. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 341. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 342. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 343. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 344. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 345. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 346. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 347. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 348. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 349. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 350. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 351. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 352. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 353. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 354. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 355. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 356. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 357. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 358. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 359. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 360. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 361. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 362. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 363. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 364. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 365. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 366. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 367. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 368. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 369. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 370. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 371. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 372. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 373. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 374. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 375. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 376. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 377. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 378. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 379. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 380. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 381. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 382. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 383. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 384. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 385. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 386. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 387. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 388. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 389. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 390. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 391. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 392. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 393. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 394. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 395. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 396. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 397. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 398. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 399. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 400. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 401. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 402. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 403. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 404. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 405. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 406. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 407. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 408. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 409. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 410. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 411. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 412. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 413. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 414. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 415. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 416. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 417. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 418. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 419. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 420. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 421. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 422. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 423. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 424. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 425. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 426. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 427. In some embodiments, the one or more non-coding RNAs detected in a sample comprise at least 70%, 75%, 80%, 85%, 90%, 91%, 92%, 93%, 94%, 95%, 96%, 97%, 98%, 99% or 100% sequence identity to SEQ ID NO: 428. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 429. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 430. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 431. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 432. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 433. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 434. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 435. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 436. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 437. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 438. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 439. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 440. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 441. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 442. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 443. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 444. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 445. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 446. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 447. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 448. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 449. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 450. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 451. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 452. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 453. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 454. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 455. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 456. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 457. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 458. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 459. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 460. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 461. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 462. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 463. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 464. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 465. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 466. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 467. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 468. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 469. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 470. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 471. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 472. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 473. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 474. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 475. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 476. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 477. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 478. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 479. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 480. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 481. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 482. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 483. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 484. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 485. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 486. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 487. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 488. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 489. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 490. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 491. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 492. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 493. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 494. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 495. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 496. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 497. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 498. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 499. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 500. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 501. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 502. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 503. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 504. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 505. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 506. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 507. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 508. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 509. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 510. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 511. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 512. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 513. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 514. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 515. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 516. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 517. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 518. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 519. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 520. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 521. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 522. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 523. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 524. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 525. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 526. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 527. In some embodiments, the one or more non-coding RNAs detected in a sample comprise at least 70%, 75%, 80%, 85%, 90%, 91%, 92%, 93%, 94%, 95%, 96%, 97%, 98%, 99% or 100% sequence identity to SEQ ID NO: 528. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 529. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 530. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 531. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 532. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 533. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 534. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 535. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 536. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 537. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 538. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 539. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 540. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 541. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 542. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 543. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 544. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 545. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 546. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 547. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 548. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 549. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 550. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 551. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 552. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 553. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 554. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 555. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 556. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 557. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 558. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 559. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 560. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 561. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 562. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 563. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 564. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 565. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 566. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 567. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 568. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 569. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 570. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 571. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 572. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 573. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 574. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 575. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 576. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 577. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 578. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 579. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 580. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 581. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 582. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 583. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 584. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 585. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 586. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 587. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 588. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 589. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 590. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 591. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 592. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 593. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 594. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 595. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 596. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 597. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 598. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 599. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 600. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 601. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 602. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 603. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 604. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 605. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 606. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 607. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 608. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 609. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 610. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 611. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 612. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 613. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 614. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 615. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 616. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 617. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 618. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 619. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 620. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 621. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 622. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 623. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 624. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 625. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 626. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 627. In some embodiments, the one or more non-coding RNAs detected in a sample comprise at least 70%, 75%, 80%, 85%, 90%, 91%, 92%, 93%, 94%, 95%, 96%, 97%, 98%, 99% or 100% sequence identity to SEQ ID NO: 628. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 629. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 630. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 631. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 632. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 633. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 634. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 635. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 636. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 637. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 638. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 639. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 640. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 641. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 642. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 643. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 644. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 645. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 646. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 647. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 648. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 649. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 650. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 651. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 652. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 653. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 654. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 655. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 656. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 657. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 658. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 659. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 660. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 661. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 662. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 663. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 664. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 665. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 666. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 667. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 668. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 669. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 670. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 671. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 672. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 673. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 674. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 675. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 676. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 677. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 678. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 679. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 680. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 681. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 682. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 683. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 684. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 685. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 686. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 687. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 688. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 689. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 690. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 691. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 692. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 693. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 694. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 695. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 696. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 697. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 698. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 699. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 700. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 701. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 702. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 703. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 704. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 705. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 706. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 707. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 708. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 709. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 710. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 711. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 712. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 713. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 714. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 715. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 716. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 717. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 718. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 719. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 720. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 721. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 722. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 723. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 724. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 725. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 726. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 727. In some embodiments, the one or more non-coding RNAs detected in a sample comprise at least 70%, 75%, 80%, 85%, 90%, 91%, 92%, 93%, 94%, 95%, 96%, 97%, 98%, 99% or 100% sequence identity to SEQ ID NO: 728. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 729. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 730. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 731. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 732. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 733. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 734. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 735. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 736. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 737. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 738. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 739. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 740. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 741. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 742. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 743. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 744. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 745. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 746. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 747. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 748. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 749. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 750. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 751. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 752. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 753. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 754. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 755. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 756. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 757. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 758. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 759. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 760. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 761. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 762. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 763. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 764. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 765. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 766. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 767. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 768. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 769. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 770. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 771. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 772. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 773. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 774. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 775. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 776. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 777. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 778. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 779. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 780. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 781. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 782. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 783. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 784. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 785. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 786. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 787. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 788. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 789. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 790. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 791. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 792. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 793. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 794. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 795. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 796. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 797. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 798. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 799. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 800. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 801. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 802. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 803. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 804. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 805. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 806. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 807. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 808. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 809. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 810. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 811. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 812. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 813. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 814. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 815. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 816. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 817. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 818. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 819. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 820. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 821. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 822. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 823. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 824. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 825. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 826. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 827. In some embodiments, the one or more non-coding RNAs detected in a sample comprise at least 70%, 75%, 80%, 85%, 90%, 91%, 92%, 93%, 94%, 95%, 96%, 97%, 98%, 99% or 100% sequence identity to SEQ ID NO: 828. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 829. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 830. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 831. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 832. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 833. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 834. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 835. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 836. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 837. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 838. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 839. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 840. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 841. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 842. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 843. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 844. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 845. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 846. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 847. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 848. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 849. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 850. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 851. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 852. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 853. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 854. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 855. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 856. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 857. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 858. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 859. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 860. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 861. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 862. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 863. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 864. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 865. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 866. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 867. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 868. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 869. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 870. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 871. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 872. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 873. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 874. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 875. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 876. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 877. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 878. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 879. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 880. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 881. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 882. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 883. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 884. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 885. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 886. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 887. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 888. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 889. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 890. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 891. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 892. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 893. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 894. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 895. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 896. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 897. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 898. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 899. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 900. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 901. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 902. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 903. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 904. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 905. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 906. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 907. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 908. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 909. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 910. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 911. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 912. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 913. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 914. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 915. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 916. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 917. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 918. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 919. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 920. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 921. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 922. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 923. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 924. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 925. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 926. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 927. In some embodiments, the one or more non-coding RNAs detected in a sample comprise at least 70%, 75%, 80%, 85%, 90%, 91%, 92%, 93%, 94%, 95%, 96%, 97%, 98%, 99% or 100% sequence identity to SEQ ID NO: 928. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 929. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 930. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 931. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 932. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 933. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 934. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 935. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 936. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 937. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 938. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 939. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 940. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 941. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 942. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 943. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 944. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 945. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 946. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 947. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 948. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 949. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 950. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 951. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 952. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 953. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 954. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 955. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 956. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 957. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 958. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 959. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 960. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 961. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 962. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 963. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 964. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 965. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 966. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 967. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 968. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 969. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 970. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 971. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 972. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 973. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 974. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 975. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 976. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 977. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 978. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 979. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 980. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 981. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 982. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 983. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 984. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 985. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 986. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 987. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 988. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 989. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 990. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 991. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 992. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 993. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 994. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 995. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 996. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 997. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 998. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 999. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1000. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1001. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1002. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1003. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1004. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1005. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1006. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1007. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1008. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1009. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1010. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1011. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1012. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1013. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1014. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1015. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1016. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1017. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1018. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1019. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1020. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1021. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1022. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1023. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1024. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1025. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1026. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1027. In some embodiments, the one or more non-coding RNAs detected in a sample comprise at least 70%, 75%, 80%, 85%, 90%, 91%, 92%, 93%, 94%, 95%, 96%, 97%, 98%, 99% or 100% sequence identity to SEQ ID NO: 1028. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1029. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1030. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1031. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1032. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1033. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1034. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1035. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1036. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1037. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1038. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1039. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1040. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1041. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1042. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1043. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1044. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1045. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1046. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1047. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1048. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1049. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1050. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1051. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1052. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1053. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1054. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1055. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1056. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1057. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1058. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1059. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1060. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1061. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1062. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1063. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1064. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1065. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1066. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1067. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1068. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1069. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1070. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1071. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1072. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1073. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1074. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1075. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1076. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1077. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1078. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1079. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1080. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1081. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1082. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1083. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1084. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1085. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1086. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1087. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1088. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1089. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1090. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1091. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1092. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1093. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1094. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1095. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1096. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1097. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1098. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1099. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1100. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1101. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1102. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1103. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1104. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1105. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1106. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1107. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1108. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1109. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1110. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1111. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1112. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1113. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1114. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1115. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1116. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1117. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1118. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1119. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1120. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1121. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1122. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1123. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1124. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1125. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1126. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1127. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1128. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1129. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1130. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO:1131. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1132. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1133. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1134. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1135. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1136. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1137. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1138. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1139. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1140. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1141. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1142. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1143. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1144. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1145. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1146. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1147. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1148. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1149. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1150. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1151. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1152. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1153. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1154. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1155. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1156. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1157. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1158. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1159. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1160. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1161. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1162. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1163. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1164. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1165. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1166. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1167. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1168. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1169. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1170. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1171. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1172. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1173. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1174. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1175. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1176. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1177. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1178. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1179. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1180. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1181. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1182. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1183. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1184. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1185. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1186. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1187. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1188. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1189. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1190. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1191. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1192. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1193. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1194. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1195. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1196. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1197. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1198. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1199. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1200. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1201. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1202. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1203. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1204. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1205. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1206. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1207. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1208. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1209. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1210. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1211. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1212. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1213. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1214. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1215. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1216. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1217. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1218. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1219. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1220. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1221. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1222. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1223. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1224. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1225. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1226. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1227. In some embodiments, the one or more non-coding RNAs detected in a sample comprise at least 70%, 75%, 80%, 85%, 90%, 91%, 92%, 93%, 94%, 95%, 96%, 97%, 98%, 99% or 100% sequence identity to SEQ ID NO: 1228. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1229. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1230. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1231. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1232. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1233. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1234. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1235. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1236. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1237. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1238. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1239. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1240. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1241. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1242. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1243. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1244. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1245. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1246. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1247. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1248. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1249. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1250. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1251. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1252. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1253. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1254. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1255. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1256. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1257. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1258. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1259. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1260. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1261. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1262. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1263. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1264. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1265. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1266. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1267. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1268. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1269. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1270. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1271. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1272. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1273. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1274. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1275. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1276. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1277. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1278. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1279. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1280. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1281. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1282. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1283. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1284. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1285. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1286. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1287. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1288. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1289. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1290. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1291. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1292. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1293. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1294. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1295. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1296. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1297. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1298. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1299. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1300. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1301. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1302. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1303. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1304. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1305. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1306. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1307. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1308. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1309. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1310. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1311. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1312. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1313. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1314. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1315. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1316. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1317. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1318. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1319. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1320. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1321. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1322. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1323. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1324. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1325. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1326. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1327. In some embodiments, the one or more non-coding RNAs detected in a sample comprise at least 70%, 75%, 80%, 85%, 90%, 91%, 92%, 93%, 94%, 95%, 96%, 97%, 98%, 99% or 100% sequence identity to SEQ ID NO: 1328. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1329. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1330. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1331. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1332. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1333. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1334. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1335. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1336. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1337. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1338. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1339. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1340. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1341. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1342. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1343. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1344. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1345. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1346. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1347. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1348. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1349. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1350. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1351. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1352. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1353. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1354. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1355. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1356. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1357. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1358. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1359. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1360. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1361. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1362. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1363. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1364. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1365. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1366. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1367. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1368. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1369. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1370. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1371. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1372. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1373. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1374. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1375. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1376. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1377. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1378. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1379. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1380. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1381. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1382. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1383. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1384. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1385. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1386. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1387. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1388. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1389. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1390. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1391. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1392. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1393. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1394. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1395. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1396. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1397. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1398. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1399. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1400. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1401. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1402. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1403. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1404. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1405. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1406. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1407. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1408. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1409. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1410. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1411. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1412. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1413. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1414. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1415. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1416. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1417. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1418. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1419. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1420. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1421. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1422. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1423. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1424. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1425. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1426. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1427. In some embodiments, the one or more non-coding RNAs detected in a sample comprise at least 70%, 75%, 80%, 85%, 90%, 91%, 92%, 93%, 94%, 95%, 96%, 97%, 98%, 99% or 100% sequence identity to SEQ ID NO: 1428. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1429. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1430. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1431. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1432. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1433. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1434. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1435. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1436. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1437. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1438. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1439. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1440. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1441. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1442. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1443. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1444. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1445. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1446. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1447. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1448. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1449. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1450. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO:1451. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1452. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1453. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1454. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1455. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1456. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1457. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1458. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1459. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1460. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1461. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1462. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1463. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1464. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1465. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1466. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1467. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1468. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1469. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1470. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1471. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1472. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1473. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1474. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1475. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1476. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1477. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1478. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1479. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1480. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1481. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1482. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1483. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1484. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1485. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1486. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1487. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1488. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1489. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1490. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1491. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1492. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1493. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1494. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1495. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1496. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1497. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1498. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1499. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1500. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1501. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1502. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1503. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1504. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1505. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1506. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1507. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1508. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1509. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1510. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1511. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1512. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1513. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1514. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1515. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1516. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1517. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1518. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1519. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1520. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1521. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1522. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1523. In some embodiments, the one or more non-coding RNAs detected in a sample comprise at least about 70%, 75%, 80%, 85%, 90%, 91%, 92%, 93%, 94%, 95%, 96%, 97%, 98%, 99% or 100% sequence identity to SEQ ID NO: 1524.

In some embodiments, the one or more non-coding RNAs detected in a sample comprise one or a plurality of functional fragments of the nucleic acid sequences disclosed in TABLE 1 (SEQ ID NO: 1 through SEQ ID NO: 1543). In some embodiments, the mere presence or expression of one or a plurality of small non-coding RNAs alone or in combination with expression of one or a plurality of the sequences in TABLE 1 (SEQ ID NO: 1 through SEQ ID NO: 1543) is indicative of the presence of cancer in a subject, particularly of the type of cancer associated with the small non-coding RNAs detected. In some embodiments, the mere presence or expression of one or a plurality of small non-coding RNAs alone or in combination with expression of one or a plurality of sequences having at least about 70%, 80%, 81%, 82%, 83%, 84, 85%, 86%, 87%, 88%, 89%, 90%, 91%, 92%, 93%, 94%, 95%, 96%, 97%, 98%, or about 99% sequence identity to any of the nucleic acid sequences in TABLE 1 (SEQ ID NO: 1 through SEQ ID NO: 1543) is indicative of the presence of cancer in a subject, particularly of the type of cancer associated with the small non-coding RNAs detected. The skilled person can readily envision additional suitable controls that may be appropriate depending on the assay in question. The aforementioned suitable controls are exemplary, and are not intended to be limiting.

Generally, an increase in the level of one or more of the small non-coding RNA biomarkers provided in TABLE 1 (SEQ ID NO: 1 through SEQ ID NO: 1543) in a biological sample of a test subject relative to a suitable control representative of the level of the same small non-coding RNA biomarkers in a normal subject will indicate that the test subject has cancer of a type and tissue of origin associated with the one or more small non-coding RNAs detected in the test subject. In some instances where the levels of two or more small non-coding RNA biomarkers are determined in a test subject, there may be an increase in the level of one or more small non-coding RNA biomarkers, and no change or an increase in the level of one or more additional small non-coding RNA biomarkers, relative to a suitable control. In such instances, a difference in the level of one or more of the small non-coding RNA biomarkers relative to a suitable control representative of the level of the small non-coding RNA biomarkers in a normal subject indicates that the test subject has cancer of a type and tissue of origin associated with the one or more small non-coding RNAs shown different level in the test subject. Determination of such a difference may be aided by the execution of a software classification algorithm, as described herein.

### Biological Samples

The expression level of one or more disclosed small non-coding RNA biomarkers may be determined in a biological sample obtained from a subject. A sample of a subject is one that originates from a subject. Such a sample may be further processed after it is obtained from the subject. For example, RNA may be isolated from a sample. In this example, the RNA isolated from the sample is also a sample obtained from the subject. A biological sample useful for determining the level of one or more disclosed small non-coding RNA biomarkers may be obtained from essentially any source, including cells, tissues, and fluids throughout the body.

In some embodiments, the biological sample used for determining the level of one or more disclosed small non-coding RNA biomarkers is a sample containing circulating small non-coding RNAs, e.g., extracellular small non-coding RNAs. Extracellular small non-coding RNAs freely circulate in a wide range of biological material, including bodily fluids, such as fluids from the circulatory system, e.g., a blood sample or a lymph sample, or from another bodily fluid such as urine or saliva. Accordingly, in some embodiments, the biological sample used for determining the level of one or more disclosed small non-coding RNA biomarkers is a bodily fluid, for example, blood, fractions thereof, serum, plasma, urine, saliva, tears, sweat, semen, vaginal secretions, lymph, bronchial secretions, CSF, whole blood, etc. In some embodiments, the sample is a sample that is obtained non-invasively. In some embodiments, the sample is a serum sample from a human. In some embodiments, the sample is a bodily fluid from a human. In some embodiments, the sample is a liquid biopsy from a human.

In some embodiments, any of the methods disclosed herein comprise using a small volume of sample for detection and/or diagnosis. In some embodiments, the disclosed methods comprise isolating total RNA and/or amplifying small non-coding RNA in a sample of no more than about 20 microliters, about 40 microliters, about 80 microliters, about 100 microliters, about 200 microliters, about 300 microliters, about 400 microliters, about 500 microliters, about 600 microliters, about 700 microliters, about 800 microliters, about 900 microliters, about 1 milliliter, about 1.1 milliliters, about 1.2 milliliters, about 1.3 milliliters, about 1.4 milliliters, about 1.5 milliliters, about 1.6 milliliters, about 1.7 milliliters, about 1.8 milliliters, about 1.9 milliliters, or about 2.0 milliliters. In some embodiments, the sample size is from about 20 microliters to about 2 milliliters of liquid sample in the form of subject plasma, whole blood or serum.

In some embodiments, the methods disclosed herein comprise isolating total RNA and/or amplifying small non-coding RNA in a sample of no more than about 20 microliters of serum, no more than about 40 microliters of serum, no more than about 80 microliters of serum, no more than about 100 microliters of serum, no more than about 200 microliters of serum, no more than about 300 microliters of serum, no more than about 400 microliters of serum, no more than about 500 microliters of serum, no more than about 600 microliters of serum, no more than about 700 microliters of serum, no more than about 800 microliters of serum, no more than about 900 microliters of serum, no more than about 1 milliliter of serum, no more than about 1.1 milliliters of serum, no more than about 1.2 milliliters of serum, no more than about 1.3 milliliters of serum, no more than about 1.4 milliliters of serum, no more than about 1.5 milliliters of serum, no more than about 1.6 milliliters of serum, no more than about 1.7 milliliters of serum, no more than about 1.8 milliliters of serum, no more than about 1.9 milliliters of serum, or no more than about 2.0 milliliters of serum.

Circulating small non-coding RNAs include small non-coding RNAs in cells, extracellular small non-coding RNAs in microvesicles, in exosomes and extracellular small non-coding RNAs that are not associated with cells or microvesicles (extracellular, non-vesicular small non-coding RNA). In some embodiments, the biological sample used for determining the level of one or more small non-coding RNA biomarkers (e.g., a sample containing circulating small non-coding RNA) may contain cells. In other embodiments, the biological sample may be free or substantially free of cells (e.g., a serum sample). In some embodiments, a sample containing circulating small non-coding RNAs, e.g., extracellular small non-coding RNAs, is a blood-derived sample. Exemplary blood-derived sample types include, e.g., a plasma sample, a serum sample, a blood sample, etc. In other embodiments, a sample containing circulating small non-coding RNAs is a lymph sample. Circulating small non-coding RNAs are also found in urine and saliva, and biological samples derived from these sources are likewise suitable for determining the level of one or more disclosed small non-coding RNA biomarkers.

In some embodiments, any of the methods of the disclosure comprises a step of isolating total RNA from a sample or cell or exosome or microvesicle. Methods of isolating RNA for expression analysis from blood, plasma and/or serum (see for example, Tsui N B et al. (2002) Clin. Chem. 48,1647-53, incorporated by reference in its entirety herein) and from urine (see for example, Boom R et al. (1990) J Clin Microbiol. 28, 495-503, incorporated by reference in its entirety herein) have been described and routinely used by the skilled person.

### Determining the Level of Small Non-Coding RNA Biomarkers in a Sample

The level of one or more disclosed small non-coding RNA biomarkers in a biological sample may be determined by any suitable method. Any reliable method for measuring the level or amount of small non-coding RNA in a sample may be used. Generally, small non-coding RNA can be detected and quantified from a sample (including fractions thereof), such as samples of isolated RNA by various methods known for mRNA, including, for example, amplification-based methods (e.g., Polymerase Chain Reaction (PCR), Real-Time Polymerase Chain Reaction (RT-PCR), Quantitative Polymerase Chain Reaction (qPCR), rolling circle amplification, etc.), hybridization-based methods (e.g., hybridization arrays (e.g., microarrays), NanoString analysis, Northern Blot analysis, branched DNA (bDNA) signal amplification, in situ hybridization, etc.), and sequencing-based methods (e.g., next-generation sequencing methods, for example, using the Illumina or IonTorrent platforms). Other exemplary techniques include ribonuclease protection assay (RPA) and mass spectroscopy.

In some embodiments, RNA is converted to DNA (cDNA) prior to analysis. cDNA can be generated by reverse transcription of isolated small non-coding RNA using conventional techniques. In some embodiments, small non-coding RNA is amplified prior to measurement. In other embodiments, the level of small non-coding RNA is measured during the amplification process. In still other embodiments, the level of small non-coding RNA is not amplified prior to measurement. Some exemplary methods suitable for determining the level of small non-coding RNA in a sample are described in greater detail below. These methods are provided by way of illustration only, and it will be apparent to a skilled person that other suitable methods may likewise be used.

Amplification-Based Methods

Many amplification-based methods exist for detecting the level of small non-coding RNA nucleic acid sequences, including, but not limited to, PCR, RT-PCR, qPCR, and rolling circle amplification. Other amplification-based techniques include, for example, ligase chain reaction, multiplex ligatable probe amplification, in vitro transcription (IVT), strand displacement amplification, transcription-mediated amplification, RNA (Eberwine) amplification, and other methods that are known to persons skilled in the art.

A typical PCR reaction includes multiple steps, or cycles, that selectively amplify target nucleic acid species: a denaturing step, in which a target nucleic acid is denatured; an annealing step, in which a set of PCR primers (i.e., forward and reverse primers) anneal to complementary DNA strands, and an elongation step, in which a thermostable DNA polymerase elongates the primers. By repeating these steps multiple times, a DNA fragment is amplified to produce an amplicon, corresponding to the target sequence. Typical PCR reactions include 20 or more cycles of denaturation, annealing, and elongation. In many cases, the annealing and elongation steps can be performed concurrently, in which case the cycle contains only two steps. A reverse transcription reaction (which produces a cDNA sequence having complementarity to a small non-coding RNA) may be performed prior to PCR amplification. Reverse transcription reactions include the use of, e.g., a RNA-based DNA polymerase (reverse transcriptase) and a primer.

The disclosure relates to compostions comprising cDNA sequences that are complementary to the full-length oncRNAs disclosed herein and appear in TABLE 2. In some embodiments, the disclosure relates to a composition comprising a fragment of a cDNA sequence provided in TABLE 2, that is from about 4 to about 200 nucleotides long and complementary to a contiguous portion of the full-length oncRNA from TABLE 2. In some embodiments, the disclosure relates to a kit comprising at least one or at least two probes that are complimentary to a portion of the oncRNAs disclosed in TABLE 2. In some embodiments, the disclosure relates to a composition comprising a fragment of a cDNA sequence provided in TABLE 2, that is from about 4 to about 300 nucleotides in length and complementary to a contiguous portion of the full-length oncRNA from TABLE 2 that is at any position nested within the full-length oncRNA or inclusive of the entire DNA sequence.

Kits for quantitative real time PCR of small non-coding RNA are known, and are commercially available. Examples of suitable kits include, but are not limited to, the TaqMan miRNA Assay (Applied Biosystems) and the mirVana. qRT-PCR miRNA detection kit (Ambion). The small non-coding RNA can be ligated to a single stranded oligonucleotide containing universal primer sequences, a polyadenylated sequence, or adaptor sequence prior to reverse transcriptase and amplified using a primer complementary to the universal primer sequence, poly(T) primer, or primer comprising a sequence that is complementary to the adaptor sequence. In some embodiments, the disclosure relates to a kit comprising at least one probe that is complementary to a portion of one oncRNA disclosed in TABLE 2. In some embodiments, the kit comprises a DNA sequence that is from TABLE 2, such DNA sequence corresponding to the oncRNA being targeted. In some embodiments, the kit comprises a DNA sequence identified in TABLE 2 or a fragment thereof having at least about 70%, 75%, 80%, 85%, 90%, 91%, 92%, 93%, 94%, 95%, 96%, 97%, 98%, or 99% sequence identity to the complementary DNA sequences of TABLE 2. In some embodiments, the aforementioned cDNA sequences can be one or two or more species of annealing oligos each independently selected from a sequence that is from about 4 to about 250 nucleotides in length nested within (e.g. is a fragment of) the DNA sequence provided in TABLE 2. In some embodiments, the cDNA sequences or fragments thereof can be utilized as probes for a PCR reaction in which they act as primers for detecting the oncRNA with which it is associated.

In some instances, custom qRT-PCR assays can be developed for determination of small non-coding RNA levels. Custom qRT-PCR assays to measure small non-coding RNAs in a biological sample, e.g., a body fluid, can be developed using, for example, methods that involve an extended reverse transcription primer and locked nucleic acid modified PCR. Custom small non-coding RNA assays can be tested by running the assay on a dilution series of chemically synthesized small non-coding RNA corresponding to the target sequence. This permits determination of the limit of detection and linear range of quantitation of each assay. Furthermore, when used as a standard curve, these data permit an estimate of the absolute abundance of small non-coding RNAs measured in biological samples.

Amplification curves may optionally be checked to verify that Ct values are assessed in the linear range of each amplification plot. Typically, the linear range spans several orders of magnitude. For each candidate small non-coding RNA assayed, a chemically synthesized version of the small non-coding RNA can be obtained and analyzed in a dilution series to determine the limit of sensitivity of the assay, and the linear range of quantitation. Relative expression levels may be determined, for example, as described by Livak et al., Methods (2001) December; 25(4):402-8.

In some embodiments, two or more small non-coding RNAs are amplified in a single reaction volume. For example, multiplex q-PCR, such as qRT-PCR, enables simultaneous amplification and quantification of at least two small non-coding RNAs of interest in one reaction volume by using more than one pair of primers and/or more than one probe. The primer pairs comprise at least one amplification primer that specifically binds each small non-coding RNA, and the probes are labeled such that they are distinguishable from one another, thus allowing simultaneous quantification of multiple small non-coding RNAs.

Rolling circle amplification is a DNA-polymerase driven reaction that can replicate circularized oligonucleotide probes with either linear or geometric kinetics under isothermal conditions (see, for example, Lizardi et al., Nat. Gen. (1998) 19(3):225-232; Gusev et al., Am. J. Pathol. (2001) 159(1):63-69; Nallur et al., Nucleic Acids Res. (2001) 29(23):E118). In the presence of two primers, one hybridizing to the (+) strand of DNA, and the other hybridizing to the (−) strand, a complex pattern of strand displacement results in the generation of over 109 copies of each DNA molecule in 90 minutes or less. Tandemly linked copies of a closed circle DNA molecule may be formed by using a single primer. The process can also be performed using a matrix-associated DNA. The template used for rolling circle amplification may be reverse transcribed. This method can be used as a highly sensitive indicator of small non-coding RNA sequence and expression level at very low small non-coding RNA concentrations (see, for example, Cheng et al., Angew Chem. Int. Ed. Engl. (2009) 48(18):3268-72; Neubacher et al., Chembiochem. (2009) 10(8):1289-91).

Hybridization-Based Methods

Small non-coding RNA may be detected using hybridization-based methods, including but not limited to hybridization arrays (e.g., microarrays), NanoString analysis, Northern Blot analysis, branched DNA (bDNA) signal amplification, and in situ hybridization.

Microarrays can be used to measure the expression levels of large numbers of small non-coding RNAs simultaneously. Microarrays can be fabricated using a variety of technologies, including printing with fine-pointed pins onto glass slides, photolithography using pre-made masks, photolithography using dynamic micromirror devices, ink-jet printing, or electrochemistry on microelectrode arrays. Also useful are microfluidic TaqMan Low-Density Arrays, which are based on an array of microfluidic qRT-PCR reactions, as well as related microfluidic qRT-PCR based methods.

Axon B-4000 scanner and Gene-Pix Pro 4.0 software or other suitable software can be used to scan images. Non-positive spots after background subtraction, and outliers detected by the ESD procedure, are removed. The resulting signal intensity values are normalized to per-chip median values and then used to obtain geometric means and standard errors for each small non-coding RNA. Each signal can be transformed to log base 2, and a one-sample t test can be conducted. Independent hybridizations for each sample can be performed on chips with each small non-coding RNA spotted multiple times to increase the robustness of the data.

Microarrays can be used for the expression profiling of small non-coding RNAs in diseases. For example, RNA can be extracted from a sample and, optionally, the small non-coding RNAs are size-selected from total RNA. Oligonucleotide linkers can be attached to the 5′ and 3′ ends of the small non-coding RNAs and the resulting ligation products are used as templates for an RT-PCR reaction. The sense strand PCR primer can have a fluorophore attached to its 5′ end, thereby labeling the sense strand of the PCR product. The PCR product is denatured and then hybridized to the microarray. A PCR product, referred to as the target nucleic acid that is complementary to the corresponding small non-coding RNA capture probe sequence on the array will hybridize, via base pairing, to the spot at which the, capture probes are affixed. The spot will then fluoresce when excited using a microarray laser scanner.

The fluorescence intensity of each spot is then evaluated in terms of the number of copies of a particular small non-coding RNA, using a number of positive and negative controls and array data normalization methods, which will result in assessment of the level of expression of a particular small non-coding RNA.

Total RNA containing the small non-coding RNA extracted from a body fluid sample can also be used directly without size-selection of the small non-coding RNAs. For example, the RNA can be 3′ end labeled using T4 RNA ligase and a fluorophore-labeled short RNA linker. Fluorophore-labeled small non-coding RNAs complementary to the corresponding small non-coding RNA capture probe sequences on the array hybridize, via base pairing, to the spot at which the capture probes are affixed. The fluorescence intensity of each spot is then evaluated in terms of the number of copies of a particular small non-coding RNA, using a number of positive and negative controls and array data normalization methods, which will result in assessment of the level of expression of a particular small non-coding RNA.

Several types of microarrays can be employed including, but not limited to, spotted oligonucleotide microarrays, pre-fabricated oligonucleotide microarrays or spotted long oligonucleotide arrays. In some embodiments, the disclosure relates to a solid support comprising silica, plastic or a combination of both silica or plastic

Small non-coding RNAs can also be detected without amplification using the nCounter Analysis System (NanoString Technologies, Seattle, Wash.). This technology employs two nucleic acid-based probes that hybridize in solution (e.g., a reporter probe and a capture probe). After hybridization, excess probes are removed, and probe/target complexes are analyzed in accordance with the manufacturer's protocol. nCounter miRNA assay kits are available from NanoString Technologies, which are capable of distinguishing between highly similar small non-coding RNAs with great specificity.

Small non-coding RNAs can also be detected using branched DNA (bDNA) signal amplification (see, for example, Urdea, Nature Biotechnology (1994), 12:926-928). Small non-coding RNA assays based on bDNA signal amplification are commercially available. One such assay is the QuantiGene® 2.0 miRNA Assay (Affymetrix, Santa Clara, Calif.).

Northern Blot and in situ hybridization may also be used to detect small non-coding RNAs. Suitable methods for performing Northern Blot and in situ hybridization are known in the art.

In some embodiments, biomarker expression is determined by an assay known to those of skill in the art, including but not limited to, multi-analyte profile test, enzyme-linked immunosorbent assay (ELISA), radioimmunoassay, Western blot assay, immunofluorescent assay, enzyme immunoassay, immunoprecipitation assay, chemiluminescent assay, immunohistochemical assay, dot blot assay, or slot blot assay. In some embodiments, wherein an antibody is used in the assay the antibody is detectably labeled. The antibody labels may include, but are not limited to, immunofluorescent label, chemiluminescent label, phosphorescent label, enzyme label, radiolabel, avidin/biotin, colloidal gold particles, colored particles, and magnetic particles. In some embodiments, biomarker expression is determined by an IHC assay.

In some embodiments, biomarker expression is determined using an agent that specifically binds the biomarker. Any molecular entity that displays specific binding to a biomarker can be employed to determine the level of that biomarker protein in a sample. Specific binding agents include, but are not limited to, antibodies, antibody fragments, antibody mimetics, and polynucleotides (e.g., aptamers). One of skill understands that the degree of specificity required is determined by the particular assay used to detect the biomarker protein. In some embodiments, the disclosure relates to a system comprising a solid support (such as an ELISA plate, gel, bead or column comprising an antibody, antibody fragment, antibody mimetic, and/or polynucleotides capable of binding to T3p or a salt thereof.

A. Sequencing-Based Methods

Advanced sequencing methods can likewise be used as available. For example, small non-coding RNAs can be detected using Illumina. Next Generation Sequencing (e.g., Sequencing-By-Synthesis or TruSeq methods, using, for example, the HiSeq, HiScan, GenomeAnalyzer, or MiSeq systems (Illumina, Inc., San Diego, Calif.)). Small non-coding RNAs can also be detected using Ion Torrent Sequencing (Ion Torrent Systems, Inc., Gulliford, Conn.), or other suitable methods of semiconductor sequencing.

Direct detection of the oncRNA can be identified in some embodiments, by sequencing the RNA and identifying the sequence as one or a fragment of the RNA sequences in Table 2.

Additional Small Non-Coding RNA Detection Tools

Mass spectroscopy can be used to quantify small non-coding RNA using RNase mapping. Isolated RNAs can be enzymatically digested with RNA endonucleases (RNases) having high specificity (e.g., RNase T1, which cleaves at the 3′-side of all unmodified guanosine residues) prior to their analysis by MS or tandem MS (MS/MS) approaches. The first approach developed utilized the on-line chromatographic separation of endonuclease digests by reversed phase HPLC coupled directly to ESI-MS. The presence of posttranscriptional modifications can be revealed by mass shifts from those expected based upon the RNA sequence. Ions of anomalous mass/charge values can then be isolated for tandem MS sequencing to locate the sequence placement of the posttranscriptionally modified nucleoside.

Matrix-assisted laser desorption/ionization mass spectrometry (MALDI-MS) has also been used as an analytical approach for obtaining information about posttranscriptionally modified nucleosides. MALDI-based approaches can be differentiated from ESI-based approaches by the separation step. In MALDI-MS, the mass spectrometer is used to separate the small non-coding RNA.

To analyze a limited quantity of intact small non-coding RNAs, a system of capillary LC coupled with nanoESI-MS can be employed, by using a linear ion trap-orbitrap hybrid mass spectrometer (LTQ Orbitrap XL, Thermo Fisher Scientific) or a tandem-quadrupole time-of-flight mass spectrometer (QSTAR XL, Applied Biosystems) equipped with a custom-made nanospray ion source, a Nanovolume Valve (Valco Instruments), and a splitless nano HPLC system (DiNa, KYA Technologies). Analyte/TEAA is loaded onto a nano-LC trap column, desalted, and then concentrated. Intact small non-coding RNAs are eluted from the trap column and directly injected into a Cl 8 capillary column, and chromatographed by RP-HPLC using a gradient of solvents of increasing polarity. The chromatographic eluent is sprayed from a sprayer tip attached to the capillary column, using an ionization voltage that allows ions to be scanned in the negative polarity mode.

Additional methods for small non-coding RNA detection and measurement include, for example, strand invasion assay (Third Wave Technologies, Inc.), surface plasmon resonance (SPR), cDNA, MTDNA (metallic DNA; Advance Technologies, Saskatoon, SK), and single-molecule methods such as the one developed by US Genomics. Multiple small non-coding RNAs can be detected in a microarray format using a novel approach that combines a surface enzyme reaction with nanoparticle-amplified SPR imaging (SPRI). The surface reaction of poly(A) polymerase creates poly(A) tails on small non-coding RNAs hybridized onto locked nucleic acid (LNA) microarrays. DNA-modified nanoparticles are then adsorbed onto the poly(A) tails and detected with SPRI. This ultrasensitive nanoparticle-amplified SPRI methodology can be used for small non-coding RNA profiling at attamole levels.

Detection of Amplified or Non-Amplified Small Non-Coding RNAs

In certain embodiments, labels, dyes, or labeled probes and/or primers are used to detect amplified or unamplified small non-coding RNAs. The skilled artisan will recognize which detection methods are appropriate based on the sensitivity of the detection method and the abundance of the target. Depending on the sensitivity of the detection method and the abundance of the target, amplification may or may not be required prior to detection. One skilled in the art will recognize the detection methods where small non-coding RNA amplification is preferred.

A probe or primer may include standard (A, T or U, G and C) bases, or modified bases. Modified bases include, but are not limited to, the AEGIS bases (from Eragen Biosciences), which have been described, e.g., in U.S. Pat. Nos. 5,432,272, 5,965,364, and 6,001,983. In certain aspects, bases are joined by a natural phosphodiester bond or a different chemical linkage. Different chemical linkages include, but are not limited to, a peptide bond or a Locked Nucleic Acid (LNA) linkage, which is described, e.g., in U.S. Pat. No. 7,060,809. In some embodiments, the disclosure relates to a system comprising a probe or primer comprising or consisting of one or a combination of DNA or cDNA sequences of Table 2. In some embodiments, the disclosure relates to a system comprising a probe or primer comprising or consisting of one or a combination of fragments of DNA or cDNA sequences of Table 2.

In a further aspect, oligonucleotide probes or primers present in an amplification reaction are suitable for monitoring the amount of amplification product produced as a function of time. In certain aspects, probes having different single stranded versus double stranded character are used to detect the nucleic acid. Probes include, but are not limited to, the 5′-exonuclease assay (e.g., TAQMAN) probes (see U.S. Pat. No. 5,538,848), stem-loop molecular beacons (see, e.g., U.S. Pat. Nos. 6,103,476 and 5,925,517), stemless or linear beacons (see, e.g., WO 9921881, U.S. Pat. Nos. 6,485,901 and 6,649,349), peptide nucleic acid (PNA) Molecular Beacons (see, e.g., U.S. Pat. Nos. 6,355,421 and 6,593,091), linear PNA beacons (see, e.g. U.S. Pat. No. 6,329,144), non-FRET probes (see, e.g., U.S. Pat. No. 6,150,097), Sunrise™/AmplifluorB™ probes (see, e.g., U.S. Pat. No. 6,548,250), stem-loop and duplex SCORPION probes (see, e.g., U.S. Pat. No. 6,589,743), bulge loop probes (see, e.g., U.S. Pat. No. 6,590,091), pseudo knot probes (see, e.g., U.S. Pat. No. 6,548,250), cyclicons (see, e.g., U.S. Pat. No. 6,383,752), MGB Eclipse™ probe (Epoch Biosciences), hairpin probes (see, e.g., U.S. Pat. No. 6,596,490), PNA light-up probes, antiprimer quench probes (Li et al., Clin. Chem. 53:624-633 (2006)), self-assembled nanoparticle probes, and ferrocene-modified probes described, for example, in U.S. Pat. No. 6,485,901.

In certain embodiments, one or more of the primers in an amplification reaction can include a label. In yet further embodiments, different probes or primers comprise detectable labels that are distinguishable from one another. In some embodiments, a nucleic acid, such as the probe or primer, may be labeled with two or more distinguishable labels.

In some aspects, a label is attached to one or more probes and has one or more of the following properties: (i) provides a detectable signal; (ii) interacts with a second label to modify the detectable signal provided by the second label, e.g., FRET (Fluorescent Resonance Energy Transfer); (iii) stabilizes hybridization, e.g., duplex formation; and (iv) provides a member of a binding complex or affinity set, e.g., affinity, antibody-antigen, ionic complexes, hapten-ligand (e.g., biotin-avidin). In still other aspects, use of labels can be accomplished using any one of a large number of known techniques employing known labels, linkages, linking groups, reagents, reaction conditions, and analysis and purification methods.

Small non-coding RNAs can be detected by direct or indirect methods. In a direct detection method, one or more small non-coding RNAs are detected by a detectable label that is linked to a nucleic acid molecule. In such methods, the small non-coding RNAs may be labeled prior to binding to the probe. Therefore, binding is detected by screening for the labeled small non-coding RNA that is bound to the probe. The probe is optionally linked to a bead in the reaction volume.

In certain embodiments, nucleic acids are detected by direct binding with a labeled probe, and the probe is subsequently detected. In some embodiments, the nucleic acids, such as amplified small non-coding RNAs, are detected using FlexMAP Microspheres (Luminex) conjugated with probes to capture the desired nucleic acids. Some methods may involve detection with polynucleotide probes modified with fluorescent labels or branched DNA (bDNA) detection, for example.

In some embodiments, biomarker expression is determined using a PCR-based assay comprising specific primers and/or probes for each biomarker. As used herein, the term “probe” refers to any molecule that is capable of selectively binding a specifically intended target biomolecule. In some embodiments, as used herein, the term “probe” refers to any molecule that may bind or associate, indirectly or directly, covalently or non-covalently, to any of the substrates and/or reaction products and/or proteases disclosed herein and whose association or binding is detectable using the methods disclosed herein. In some embodiments, the probe is a fluorogenic probe, antibody or absorbance-based probes. If an absorbance-based probe, the chromophore pNA (para-nitroaniline) may be used as a probe for detection and/or quantification of a target nucleic acid sequence disclosed herein. In some embodiments, the probe may comprise a nucleic acid sequence labeled with a fluorogenic molecule or a substrate that when exposed to an enzyme becomes fluorogenic and the nucleic acid sequence is complementary to any of the nucleic acid sequences disclosed in TABLE 1 or one comprising at least about 70%, 80%, 81%, 82%, 83%, 84, 85%, 86%, 87%, 88%, 89%, 90%, 91%, 92%, 93%, 94%, 95%, 96%, 97%, 98% or 99% sequence identity to any of the nucleic acid sequences disclosed in TABLE 1 (SEQ ID NO: 1 through SEQ ID NO: 1543). Probes can be synthesized by one of skill in the art using known techniques, or derived from biological preparations. Probes may include but are not limited to, RNA, DNA, proteins, peptides, aptamers, antibodies, and organic molecules. The term “primer” or “probe” encompasses oligonucleotides that have a specific sequence or oligoribonucleotides that have a specific sequence.

The target molecule could be any one or a combination of nucleic acid sequences identified in TABLE 1, or any one or combination of nucleic acid sequences chosen from SEQ ID NO: 1 through SEQ ID NO: 1543. In some embodiments, the target molecule is a nucleic acid sequence comprising at least about 70%, 80%, 81%, 82%, 83%, 84, 85%, 86%, 87%, 88%, 89%, 90%, 91%, 92%, 93%, 94%, 95%, 96%, 97%, 98%, or about 99% sequence identity to any one or combination of nucleic acid sequences provided in TABLE 1, or any one or combination of nucleic acid sequences chosen from SEQ ID NO: 1 through SEQ ID NO: 1543. In some embodiments, the target molecule is any amplified fragment of any one or combination of nucleic acid sequences identified in TABLE 2, or any one or combination of nucleic acid sequences chosen from SEQ ID NO: 1544 through SEQ ID NO: 6843, and/or any one or combination of nucleic acid sequence comprising at least about 70%, 80%, 81%, 82%, 83%, 84, 85%, 86%, 87%, 88%, 89%, 90%, 91%, 92%, 93%, 94%, 95%, 96%, 97%, 98%, or about 99% sequence identity to any one or combination of nucleic acid sequences in TABLE 2, or any one or combination of nucleic acid sequences chosen from SEQ ID NO: 1544 through SEQ ID NO: 6843. The target molecule could be any one or a combination of nucleic acid sequences identified in TABLE 2, or any one or combination of nucleic acid sequences chosen from SEQ ID NO: 1544 through SEQ ID NO: 18. In some embodiments, the target molecule is a nucleic acid sequence comprising at least about 70%, 80%, 81%, 82%, 83%, 84, 85%, 86%, 87%, 88%, 89%, 90%, 91%, 92%, 93%, 94%, 95%, 96%, 97%, 98%, or about 99% sequence identity to any one or combination of nucleic acid sequences provided in TABLE 1, or any one or combination of nucleic acid sequences chosen from SEQ ID NO: 1 through SEQ ID NO: 1543

In other embodiments, nucleic acids are detected by indirect detection methods. For example, a biotinylated probe may be combined with a streptavidin-conjugated dye to detect the bound nucleic acid. The streptavidin molecule binds a biotin label on amplified small non-coding RNA, and the bound small non-coding RNA is detected by detecting the dye molecule attached to the streptavidin molecule. In some embodiments, the streptavidin-conjugated dye molecule comprises PHYCOLINK. Streptavidin R-Phycoerythrin (PROzyme). Other conjugated dye molecules are known to persons skilled in the art.

Labels include, but are not limited to, light-emitting, light-scattering, and light-absorbing compounds which generate or quench a detectable fluorescent, chemiluminescent, or bioluminescent signal (see, e.g., Kricka, L., Nonisotopic DNA Probe Techniques, Academic Press, San Diego (1992) and Garman A., Non-Radioactive Labeling, Academic Press (1997)). A dual labeled fluorescent probe that includes a reporter fluorophore and a quencher fluorophore is used in some embodiments. It will be appreciated that pairs of fluorophores are chosen that have distinct emission spectra so that they can be easily distinguished.

In certain embodiments, labels are hybridization-stabilizing moieties which serve to enhance, stabilize, or influence hybridization of duplexes, e.g., intercalators and intercalating dyes (including, but not limited to, ethidium bromide and SYBR-Green), minor-groove binders, and cross-linking functional groups (see, e.g., Blackburn et al., eds. “DNA and RNA Structure” in Nucleic Acids in Chemistry and Biology (1996)).

In other embodiments, methods relying on hybridization and/or ligation to quantify small non-coding RNAs may be used, including oligonucleotide ligation (OLA) methods and methods that allow a distinguishable probe that hybridizes to the target nucleic acid sequence to be separated from an unbound probe. As an example, HARP-like probes, as disclosed in U.S. Publication No. 2006/0078894 may be used to measure the quantity of miRNAs. In such methods, after hybridization between a probe and the targeted nucleic acid, the probe is modified to distinguish the hybridized probe from the unhybridized probe. Thereafter, the probe may be amplified and/or detected. In general, a probe inactivation region comprises a subset of nucleotides within the target hybridization region of the probe. To reduce or prevent amplification or detection of a HARP probe that is not hybridized to its target nucleic acid, and thus allow detection of the target nucleic acid, a post-hybridization probe inactivation step is carried out using an agent which is able to distinguish between a HARP probe that is hybridized to its targeted nucleic acid sequence and the corresponding unhybridized HARP probe. The agent is able to inactivate or modify the unhybridized HARP probe such that it cannot be amplified. A probe ligation reaction may also be used to quantify small non-coding RNAs. In a Multiplex Ligation-dependent Probe Amplification (MLPA) technique (Schouten et al., Nucleic Acids Research 30:e57 (2002)), pairs of probes which hybridize immediately adjacent to each other on the target nucleic acid are ligated to each other driven by the presence of the target nucleic acid. In some aspects, MLPA probes have flanking PCR primer binding sites. MLPA probes are specifically amplified when ligated, thus allowing for detection and quantification of small non-coding RNA biomarkers.

### Detecting a Level of Small Non-Coding RNA Biomarkers

The small non-coding RNA biomarkers described herein can be used individually or in combination in diagnostic tests to assess the type of cancer, tissue of origin, and status or stage of the cancer in a subject. Cancer status or stage includes the presence or absence of the cancer. Cancer status or stage may also include monitoring the course of the cancer, for example, monitoring disease progression. Based on the cancer status or stage of a subject, additional procedures may be indicated, including, for example, additional diagnostic tests or therapeutic procedures.

The power of a diagnostic test to correctly predict disease status is commonly measured in terms of the accuracy of the assay, the sensitivity of the assay, the specificity of the assay, or the “Area Under a Curve” (AUC), for example, the area under a Receiver Operating Characteristic (ROC) curve. As used herein, accuracy is a measure of the fraction of misclassified samples. Accuracy may be calculated as the total number of correctly classified samples divided by the total number of samples, e.g., in a test population. Sensitivity is a measure of the “true positives” that are predicted by a test to be positive, and may be calculated as the number of correctly identified breast cancer samples divided by the total number of breast cancer samples. Specificity is a measure of the “true negatives” that are predicted by a test to be negative, and may be calculated as the number of correctly identified normal samples divided by the total number of normal samples. AUC is a measure of the area under a Receiver Operating Characteristic curve, which is a plot of sensitivity vs. the false positive rate (1-specificity). The greater the AUC, the more powerful the predictive value of the test. Other useful measures of the utility of a test include the “positive predictive value,” which is the percentage of actual positives who test as positives, and the “negative predictive value,” which is the percentage of actual negatives who test as negatives. In some embodiments, the level of one or more small non-coding RNA biomarkers in samples obtained from subjects having different cancer statuses show a statistically significant difference of at least about 0.05 (p=0.05) relative to normal subjects, as determined relative to a suitable control. In some embodiments, the level of one or more small non-coding RNA biomarkers in samples obtained from subjects having different cancer statuses show a statistically significant difference of at least about 0.01 (p=0.01) relative to normal subjects, as determined relative to a suitable control. In some embodiments, the level of one or more small non-coding RNA biomarkers in samples obtained from subjects having different cancer statuses show a statistically significant difference of at least about 0.005 (p=0.005) relative to normal subjects, as determined relative to a suitable control. In some embodiments, the level of one or more small non-coding RNA biomarkers in samples obtained from subjects having different cancer statuses show a statistically significant difference of at least about 0.001 (p=0.001) relative to normal subjects, as determined relative to a suitable control.

In other embodiments, diagnostic tests that use small non-coding RNA biomarkers described herein individually or in combination show an accuracy of at least about 75%, e.g., an accuracy of at least about 75%, about 80%, about 85%, about 90%, about 95%, about 97%, about 99% or about 100%. In other embodiments, diagnostic tests that use small non-coding RNA biomarkers described herein individually or in combination show a specificity of at least about 75%, e.g., a specificity of at least about 75%, about 80%, about 85%, about 90%, about 95%, about 97%, about 99% or about 100%. In other embodiments, diagnostic tests that use small non-coding RNA biomarkers described herein individually or in combination show a sensitivity of at least about 75%, e.g., a sensitivity of at least about 75%, about 80%, about 85%, about 90%, about 95%, about 97%, about 99% or about 100%. In other embodiments, diagnostic tests that use small non-coding RNA biomarkers described herein individually or in combination show a specificity and sensitivity of at least about 75% each, e.g., a specificity and sensitivity of at least about 75%, about 80%, about 85%, about 90%, about 95%, about 97%, about 99% or about 100% (for example, a specificity of at least about 80% and sensitivity of at least about 80%, or for example, a specificity of at least about 80% and sensitivity of at least about 95%).

Each biomarker listed in TABLE 1 (SEQ ID NO: 1 through SEQ ID NO: 1543) is identified as being associated with certain type(s) of cancer as provided. In some instances, one particular small non-coding RNA biomarker may be associated with more than one types of cancers (e.g., SEQ ID NO: 1). In other instances, one particular small non-coding RNA biomarker may be associated with only one type of cancer (e.g., SEQ ID NO: 28).

Each biomarker listed in TABLE 1 (SEQ ID NO: 1 through SEQ ID NO: 1543) is differentially present in biological samples derived from subjects having certain types of cancers as compared with normal subjects, and thus each is individually useful in facilitating the determination of those types of cancer in a test subject. Such a method involves determining the level of the biomarker in a sample obtained from the subject. Determining the level of the biomarker in a sample may include measuring, detecting, or assaying the level of the biomarker in the sample using any suitable method, for example, the methods set forth herein. Determining the level of the biomarker in a sample may also include examining the results of an assay that measured, detected, or assayed the level of the biomarker in the sample. The method may also involve comparing the level of the biomarker in a sample with a suitable control. A change in the level of the biomarker relative to that in a normal subject as assessed using a suitable control is indicative of the cancer status or stage of the subject. A diagnostic amount of a biomarker that represents an amount of the biomarker above or below which a subject is classified as having a particular cancer status or stage can be used. For example, if the biomarker is upregulated in samples from an individual having cancer as compared to a normal individual, a measured amount above the diagnostic cutoff provides a diagnosis of the type of cancer that individual has. Generally, the individual small non-coding RNA biomarkers in TABLE 1 (SEQ ID NO: 1 through SEQ ID NO: 1543) are upregulated in cancer samples relative to samples obtained from normal individuals. As is well-understood in the art, adjusting the particular diagnostic cut-off used in an assay allows one to adjust the sensitivity and/or specificity of the diagnostic assay as desired. The particular diagnostic cut-off can be determined, for example, by measuring the amount of the biomarker in a statistically significant number of samples from subjects with different cancer statuses, and drawing the cut-off at the desired level of accuracy, sensitivity, and/or specificity. In certain embodiments, the diagnostic cut-off can be determined with the assistance of a classification algorithm, as described herein. In some embodiments, the

Accordingly, methods are provided for diagnosing cancer in a subject, by determining the level of at least one small non-coding RNA in a sample from the subject, wherein a difference in the level of the at least one small non-coding RNA versus that in a normal subject (as determined relative to a suitable control) is indicative of cancer in the subject. In some embodiments, the at least one small non-coding RNA includes one or more small non-coding RNAs from TABLE 1 (SEQ ID NO: 1 through SEQ ID NO: 1543). In some embodiments, the at least one small non-coding RNA includes one or more small non-coding RNAs from TABLE 2 (SEQ ID NO: 1544 through SEQ ID NO: 6569). In some embodiments, a difference in the level of the at least one small non-coding RNA versus that in a normal subject (as determined relative to a suitable control) is indicative of the type(s) of cancer identified as being associated with the detected at least one small non-coding RNA in the subject. For example, the disclosed method of determining the level of at least one small non-coding RNA in a sample from a subject, wherein an increase in the level of the at least one small non-coding RNA relative to a control is indicative of cancer in the subject, particularly of the type(s) of cancer identified as being associated with the at least one small non-coding RNA detected.

Optionally, the method may further comprise providing a diagnosis that the subject has or does not have cancer based on the level of at least one small non-coding RNA in the sample. In addition or alternatively, the method may further comprise correlating a difference in the level or levels of at least one small non-coding RNA relative to a suitable control with a diagnosis of cancer in the subject. In some embodiments, such a diagnosis may be provided directly to the subject, or it may be provided to another party involved in the subject's care.

While individual small non-coding RNA biomarkers are useful in diagnostic applications for various types of cancer, as shown herein, a combination of small non-coding RNA biomarkers may provide greater predictive value of cancer status or stage than the small non-coding RNA biomarkers when used alone. Specifically, the detection of a plurality of small non-coding RNA biomarkers can increase the accuracy, sensitivity, and/or specificity of a diagnostic test. The detection of a plurality of small non-coding RNA biomarkers can also assist in narrowing down the type of cancer and/or status or stage thereof in a subject. This is particular useful when a given small non-coding RNA biomarker is identified as being associated with more than one type of cancer. For instance, if RNA biomarker A is identified as being associated with cancers X, Y and Z, RNA biomarker B is identified as being associated with cancers X and Y, and RNA biomarker C is identified as being associated with cancers X and Z, by a process of elimination, a detection of the presence of RNA biomarkers A, B and C in a subject is indicative that the subject has cancer X. The disclosure thus includes the individual RNA biomarker provided in TABLE 1 (SEQ ID NO: 1 through SEQ ID NO: 1543) and biomarker combinations as set forth herein, and their use in methods and kits described herein.

Accordingly, methods are provided for diagnosing cancer in a subject, by determining the level of two or more small non-coding RNAs in a sample from the subject, wherein a difference in the level of the small non-coding RNAs versus that in a normal subject (as determined relative to a suitable control) is indicative of cancer in the subject. In some embodiments, the small non-coding RNAs include one or more of small non-coding RNAs provided in TABLE 1 (SEQ ID NO: 1 through SEQ ID NO: 1543). In some embodiments, the type(s) of cancer thus diagnosed is/are the one(s) provided in TABLE 1 as being associated with each individual small non-coding RNA provided in TABLE 1 (SEQ ID NO: 1 through SEQ ID NO: 1543). In some embodiments, the type(s) of cancer thus diagnosed is/are the one(s) provided in TABLE 2 as being associated with each individual small non-coding RNA provided in TABLE 2 (SEQ ID NO: 1544 through SEQ ID NO: 6834).

Also provided is a method of diagnosing cancer in a subject by determining the levels of two or more small non-coding RNAs in a sample from the subject, comparing the levels of the two or more small non-coding RNAs in the sample to a set of data representing levels of the same small non-coding RNAs present in normal subjects and subjects having a particular type of cancer, and diagnosing the subject as having or not having that particular type of cancer based on the comparison. In such a method, the set of data serves as a suitable control or reference standard for comparison with the sample from the subject.

Comparison of the sample from the subject with the set of data may be assisted by a classification algorithm, which computes whether or not a statistically significant difference exists between the collective levels of the two or more small non-coding RNAs in the sample, and the levels of the same small non-coding RNAs present in normal subjects or subjects having cancer.

### Generation of Classification Algorithms for Qualifying Cancer Status

In some embodiments, data that are generated using samples such as “known samples” can then be used to “train” a classification model. A “known sample” is a sample that has been pre-classified, e.g., classified as being derived from a normal subject or from a subject having a particular type of cancer. The data that are derived from the spectra and are used to form the classification model can be referred to as a “training data set.” Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased versus non-diseased).

In some embodiments, data for the training data set that is used to form the classification model can be obtained directly from quantitative PCR (for example, Ct values obtained using the double delta Ct method), or from high-throughput expression profiling, such as microarray analysis (for example, total counts or normalized counts from a small non-coding RNA expression assay).

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review,” IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines).

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm. Learning algorithms asserted for use in classifying biological information are described, for example, in PCT International Publication No. WO 01/31580 (Barnhill et al., “Methods and devices for identifying patterns in biological systems and methods of use thereof”), U.S. application publication No. 2002/0193950 A1 (Gavin et al, “Method or analyzing mass spectra”), U.S. application publication No. 2003/0004402 A1 (Hitt et al., “Process for discriminating between biological states based on hidden patterns from biological data”), and U.S. application publication No. 2003/0055615 A1 (Zhang and Zhang, “Systems and methods for processing biological expression data”). The contents of the foregoing patent applications are incorporated herein by reference in their entireties.

The classification models can be formed on and used on any suitable digital computer. Suitable digital computers include micro, mini, or large computers using any standard or specialized operating system, such as a Unix, WINDOWS or LINUX based operating system.

The training data set(s) and the classification models can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

The learning algorithms described above can be used for developing classification algorithms for small non-coding RNA biomarkers for various types of cancer. The classification algorithms can, in turn, be used in diagnostic tests by providing diagnostic values (e.g., cut-off points) for biomarkers used singly or in combination.

### Additional Diagnostic Tests

The level of small non-coding RNA biomarkers indicative of various types of cancer may be used as a stand-alone diagnostic indicator of cancer in a subject. Optionally, the methods may include the performance of at least one additional test to facilitate the diagnosis of cancer. For example, other tests in addition to determining the level of one or more small non-coding RNA biomarkers in order to facilitate a diagnosis of cancer may be performed. Any other test or combination of tests used in clinical practice to facilitate a diagnosis of cancer may be used in conjunction with the small non-coding RNA biomarkers described herein.

### Methods of Treatment

In some embodiments, where a subject is diagnosed with a particular type of cancer by the methods described herein, the present disclosure further provides methods of treating the subject identified to have cancer. Accordingly, in some embodiments, the disclosure relates to a method of treating cancer in a subject, comprising determining the level of at least one small non-coding RNA biomarker in a sample from the subject, wherein a difference in the level of at least one small non-coding RNA biomarker versus that in a normal subject as determined relative to a suitable control is indicative of cancer in the subject, and administering a therapeutically effective amount of a cancer therapeutic to the subject. In another embodiments, the disclosure relates to a method of treating a subject having cancer, comprising identifying a subject having cancer in which the level of at least one small non-coding RNA biomarker in a sample from the subject is different (e.g., increased) versus that in a normal subject as determined relative to a suitable control, and administering a therapeutically effective amount of a cancer therapeutic to the subject.

In some embodiments, the disclosure relates to a method of treating lung cancer in a subject, comprising determining the level of at least one small non-coding RNA biomarker chosen from SEQ ID NO: 121 and 241-593 in a sample from the subject, wherein a difference in the level of the at least one small non-coding RNA biomarker versus that in a normal subject as determined relative to a suitable control is indicative of lung cancer in the subject, and administering a therapeutically effective amount of a cancer therapeutic to the subject. In some embodiments, the disclosure relates to a method of treating breast cancer in a subject, comprising determining the level of at least one small non-coding RNA biomarker chosen from SEQ ID NO: 1-11 in a sample from the subject, wherein a difference in the level of the at least one small non-coding RNA biomarker versus that in a normal subject as determined relative to a suitable control is indicative of breast cancer in the subject, and administering a therapeutically effective amount of a cancer therapeutic to the subject. In some embodiments, the disclosure relates to a method of treating prostate cancer in a subject, comprising determining the level of at least one small non-coding RNA biomarker chosen from SEQ ID NO: 1367 through SEQ ID NO: 1423 in a sample from the subject, wherein a difference in the level of the at least one small non-coding RNA biomarker versus that in a normal subject as determined relative to a suitable control is indicative of prostate cancer in the subject, and administering a therapeutically effective amount of a cancer therapeutic to the subject. In some embodiments, the disclosure relates to a method of treating colon cancer in a subject, comprising determining the level of at least one small non-coding RNA biomarker chosen from SEQ ID NO: 12-172 in a sample from the subject, wherein a difference in the level of the at least one small non-coding RNA biomarker versus that in a normal subject as determined relative to a suitable control is indicative of colon cancer in the subject, and administering a therapeutically effective amount of a cancer therapeutic to the subject. In some embodiments, the disclosure relates to a method of treating rectal cancer in a subject, comprising determining the level of at least one small non-coding RNA biomarker chosen from SEQ ID NO: 1191 and 1424-1543 in a sample from the subject, wherein a difference in the level of the at least one small non-coding RNA biomarker versus that in a normal subject as determined relative to a suitable control is indicative of rectal cancer in the subject, and administering a therapeutically effective amount of a cancer therapeutic to the subject. In some embodiments, the disclosure relates to a method of treating pancreatic cancer in a subject, comprising determining the level of at least one small non-coding RNA biomarker chosen from SEQ ID NO: 261, 264, 315, 494 and 1057-1366 in a sample from the subject, wherein a difference in the level of the at least one small non-coding RNA biomarker versus that in a normal subject as determined relative to a suitable control is indicative of pancreatic cancer in the subject, and administering a therapeutically effective amount of a cancer therapeutic to the subject. In some embodiments, the disclosure relates to a method of treating liver cancer in a subject, comprising determining the level of at least one small non-coding RNA biomarker chosen from SEQ ID NO: 148 and 173-240 in a sample from the subject, wherein a difference in the level of the at least one small non-coding RNA biomarker versus that in a normal subject as determined relative to a suitable control is indicative of liver cancer in the subject, and administering a therapeutically effective amount of a cancer therapeutic to the subject. In some embodiments, the disclosure relates to a method of treating ovarian cancer in a subject, comprising determining the level of at least one small non-coding RNA biomarker chosen from SEQ ID NO: 594-1056 in a sample from the subject, wherein a difference in the level of the at least one small non-coding RNA biomarker versus that in a normal subject as determined relative to a suitable control is indicative of ovarian cancer in the subject, and administering a therapeutically effective amount of a cancer therapeutic to the subject. In some embodiments, the methods of the disclosure comprise a step of detecting, wherein the step of detecting the oncRNA is detected by sequencing isolated RNA from a sample or by amplifying a reverse transcribed DNA corresponding to the oncRNA in the sample.

The term “cancer therapeutic” includes, for example, substances approved by the U.S. Food and Drug Administration for the treatment of cancer. For instance, drugs approved to treat breast cancer include, but are not limited to, Abemaciclib, Abitrexate (Methotrexate), Abraxane (Paclitaxel Albumin-stabilized Nanoparticle Formulation), Ado-Trastuzumab Emtansine, Afinitor (Everolimus), Anastrozole, Aredia (Pamidronate Disodium), Arimidex (Anastrozole), Aromasin (Exemestane), Capecitabine, Clafen (Cyclophosphamide), Cyclophosphamide, Cytoxan (Cyclophosphamide), Docetaxel, Doxorubicin Hydrochloride, Ellence (Epirubicin Hydrochloride), Epirubicin Hydrochloride, Eribulin Mesylate, Everolimus, Exemestane, 5-FU (Fluorouracil Injection), Fareston (Toremifene), Faslodex (Fulvestrant), Femara (Letrozole), Fluorouracil Injection, Folex (Methotrexate), Folex PFS (Methotrexate), Fulvestrant, Gemcitabine Hydrochloride, Gemzar (Gemcitabine Hydrochloride), Goserelin Acetate, Halaven (Eribulin Mesylate), Herceptin (Trastuzumab), Ibrance (Palbociclib), Ixabepilone, Ixempra (Ixabepilone), Kadcyla (Ado-Trastuzumab Emtansine), Kisqali (Ribociclib), Lapatinib, Ditosylate, Letrozole, Megestrol Acetate, Methotrexate, Methotrexate LPF (Methotrexate), Mexate (Methotrexate), Mexate-AQ (Methotrexate), Neosar (Cyclophosphamide), Neratinib Maleate, Nerlynx (Neratinib Maleate), Nolvadex (Tamoxifen Citrate), Paclitaxel, Paclitaxel Albumin-stabilized Nanoparticle Formulation, Palbociclib, Pamidronate Disodium, Perjeta (Pertuzumab), Pertuzumab, Ribociclib, Tamoxifen Citrate, Taxol (Paclitaxel), Taxotere (Docetaxel), Thiotepa, Toremifene, Trastuzumab, Tykerb (Lapatinib Ditosylate), Velban (Vinblastine Sulfate), Velsar (Vinblastine Sulfate), Verzenio (Abemaciclib), Vinblastine Sulfate, Xeloda (Capecitabine), Zoladex (Goserelin Acetate).

The cancer therapeutics may be administered to a subject using a pharmaceutical composition. Suitable pharmaceutical compositions comprise a pharmaceutically effective amount of a cancer therapeutic (or a pharmaceutically acceptable salt or ester thereof), and optionally comprise a pharmaceutically acceptable carrier. In certain embodiments, these compositions optionally further comprise one or more additional therapeutic agents.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds, or separately by reacting a free base or free acid function with a suitable reagent. For example, a free base function can be reacted with a suitable acid. Furthermore, where the compounds carry an acidic moiety, suitable pharmaceutically acceptable salts thereof may, include metal salts such as alkali metal salts, e.g., sodium or potassium salts, and alkaline earth metal salts, e.g., calcium or magnesium salts. In some embodiments, the cancer therapeutic is a pharmaceutically acceptable salt.

The term “pharmaceutically acceptable ester,” as used herein, refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. In some embodiments, the cancer therapeutic is a pharmaceutically acceptable ester.

As described above, the pharmaceutical compositions may additionally comprise a pharmaceutically acceptable carrier. The term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, suitable for preparing the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar, buffering agents such as magnesium hydroxide and aluminum hydroxide; alginic acid; pyrogen free water, isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Compositions for use in the present disclosure may be formulated to have any concentration of the cancer therapeutic desired. In some embodiments, the composition is formulated such that it comprises a therapeutically effective amount of the cancer therapeutic.

The disclosure generally relates to a method of diagnosing a subject with a benign, pre-malignant, or malignant hyperproliferative cell comprising: detecting the presence, absence, and/or quantity of at least one non-coding RNA or functional fragment thereof in a sample. In some embodiments, the step of detecting comprise exposing a sample from a subject (e.g., a human subject) to one or a plurality of probes, each probe capable of binding one or a plurality of non-coding RNA molecules in the sample. In some embodiments, the probe is a labeled nucleic acid molecule (DNA, RNA or hybrid thereof) that comprises at least about 70%, 80%, 85%, 90%, 95%, 96%, 97%, 98%, 99% or 100% sequence identity to the complement of any nucleic acid sequences of TABLE 1 (SEQ ID NO: 1 through SEQ ID NO: 1543). In some embodiments, the probe is a labeled nucleic acid molecule (DNA, RNA or hybrid thereof) that is an RNA sequence comprising at least about 70%, 80%, 85%, 90%, 95%, 96%, 97%, 98%, 99% or 100% sequence identity to the complement of any nucleic acid sequences of TABLE 1 (SEQ ID NO: 1 through SEQ ID NO: 1543), where each thymine is replaced with a uracil. In some embodiments, the probe is a labeled nucleic acid molecule (DNA, RNA or hybrid thereof) that comprises at least about 70%, 80%, 85%, 90%, 95%, 96%, 97%, 98%, 99% or 100% sequence identity to the DNA sequences of TABLE 2. In some embodiments, the plurality of probes are one or a combination of labeled nucleic acid sequences that are an RNA or DNA complementary to a nucleic acid sequence comprising at least about 70%, 80%, 85%, 90%, 95%, 96%, 97%, 98%, 99% or 100% sequence identity to any nucleic acid sequences of TABLE 2 (SEQ ID NO: 1544 through SEQ ID NO: 18676). In some embodiments, the plurality of probes are one or a combination of labeled nucleic acid sequences that are an RNA or DNA complementary to a nucleic acid sequence comprising at least about 70%, 80%, 85%, 90%, 95%, 96%, 97%, 98%, 99% or 100% sequence identity to any nucleic acid sequences of TABLE 1 (SEQ ID NO: 1 through SEQ ID NO: 1543). In some embodiments, the plurality of probes are one or a combination of labeled nucleic acid sequences chosen from any nucleic acid sequences of TABLE 1 (SEQ ID NO: 1 through SEQ ID NO: 1543). In some embodiments, the plurality of probes comprise one or a combination of nucleic acid sequences complementary to the nucleic acid sequences chosen from any nucleic acid sequences of TABLE 1 or TABLE 2 (SEQ ID NO: 1 through SEQ ID NO:18676).

In any of the disclosed method embodiments, the subject may be a human diagnosed with or suspected as having cancer. In any of the disclosed method embodiments, wherein the step of detecting is preceded by a step of acquiring a sample from the subject.

In some embodiments, the probe or plurality of probes are one or a plurality of antibodies or antibody fragments comprising a CDR that binds to a nucleic acid molecule (DNA, RNA or hybrid thereof) that comprises at least 70%, 80%, 85%, 90%, 95%, 96%, 97%, 98%, 99% or 100% sequence identity to any nucleic acid sequences of TABLE 1 (SEQ ID NO: 1 through SEQ ID NO: 18676). In some embodiments, the probe or plurality of probes are one or a plurality of antibodies or antibody fragments comprising a CDR that binds to a nucleic acid molecule (DNA, RNA or hybrid thereof) that comprises at least about 70%, 80%, 85%, 90%, 95%, 96%, 97%, 98%, 99% or 100% sequence identity to any nucleic acid sequences of TABLE 1 (SEQ ID NO: 1 through SEQ ID NO: 18676), wherein each of sequences are modified such that the thymines in each sequence are replaced with a uracil. In some of the embodiments, the methods further comprise isolating RNA from the sample before exposing the sample to one or a plurality of probes. In some embodiments, the method comprises detecting or quantifying an amount of non-coding RNAs, such as small RNAs (smRNAs), in a sample by performing semiquantitative or quantitative PCR or sequencing analysis of the non-coding RNAs in a sample. Probes may be immobilized to a solid support such as an ELISA plate, plastic, slide, microarray, silica chip or other surface such that the single-strand nucleotide sequences are exposed to a sample comprising non-coding RNAs from a subject. The probes may comprise, in some embodiments, from about 5 to bout 100 nucleotides in length and comprise any of the sequences provided in TABLE 1 (SEQ ID NO: 1 through SEQ ID NO: 1543) or any complementary sequence in RNA or DNA form of the sequences set forth in TABLE 1 (SEQ ID NO: 1 through SEQ ID NO: 1543). In any of the disclosed method embodiments, the step of detecting the presence, absence, and/or quantity of at least one small non-coding RNA or functional fragments thereof having at least about 70% sequence identity to one of the non-coding RNAs in a sample comprises using a chemoluminescent probe, fluorescent probe, and/or fluorescence microscopy, calculating the presence or quantity by correlating the signal of the detectable probe to the presence of the non-coding RNA.

In some embodiments, any of the methods disclosed herein further comprise a step of correlating the presence or quantity of one or more small non-coding RNAs such as those disclosed in TABLE 1 (SEQ ID NO: 1 through SEQ ID NO: 1543) or any combination thereof to the likelihood that the subject has cancer. In some embodiments, any of the methods disclosed herein further comprise a step of correlating the presence or quantity of one or more small non-coding RNAs such as those disclosed in TABLE 2 (SEQ ID NO: 1544 through SEQ ID NO: 6834) or any combination thereof to the likelihood that the subject has cancer In some embodiments, the disclosure relates to a method of preparing, isolating or assessing a ribonucleic acid (RNA) fraction from a subject useful for analyzing a miRNA involved in cancer comprising: extracting RNA from a substantially cell-free sample of blood plasma or blood serum of a subject to obtain RNA pools; (b) producing a fraction of the RNA extracted in (a) by: (i) sequence discrimination of the RNA; and (ii) selectively removing non-coding RNA by exposing one or a plurality of probes to the non-coding RNA, wherein the non-coding RNA after (b) comprises one or a plurality of RNAs disclosed in TABLE 1; and (c) analyzing the non-coding RNA in the fraction of RNA produced in (b). In some embodiments, the step of analyzing comprises normalizing the amount of non-coding RNA in the sample as compared to a control amount of non-coding RNA from a control sample and determining whether the subject has cancer by comparing the normalized presence, absence or quantity of non-coding RNA in the sample to presence, absence or quantity of non-coding RNA in a control sample.

### Kits for Detection Small RNA Biomarkers

In another aspect, the present disclosure provides kits for diagnosing type of cancer, tissue of origin, and status or stage of the cancer in a subject, which kits are useful for determining the level of one or more small non-coding RNA biomarkers from TABLE 1 (SEQ ID NO: 1 through SEQ ID NO: 1543; wherein the sequences optionally comprise uracils in place of one, more than one, or all of the disclosed thymines), and combinations thereof. In some embodiments, the one or more small non-coding RNAs are selected from the biomarkers listed in TABLE 1 (SEQ ID NO: 1 through SEQ ID NO: 1543). Kits may include materials and reagents adapted to selectively detect the presence of a small non-coding RNA or group of small non-coding RNAs diagnostic for cancer in a sample of a subject. For example, in one embodiment, the kit may include a reagent that specifically hybridizes to a small non-coding RNA. Such a reagent may be a nucleic acid molecule in a form suitable for detecting the small non-coding RNA, for example, a probe or a primer. The kit may include reagents useful for performing an assay to detect one or more small non-coding RNAs, for example, reagents which may be used to detect one or more small non-coding RNAs in a qPCR reaction. The kit may likewise include a microarray useful for detecting one or more small non-coding RNAs.

In some embodiments, the kit may contain instructions for suitable operational parameters in the form of a label or product insert. For example, the instructions may include information or directions regarding how to collect a sample, how to determine the level of one or more small non-coding RNA biomarkers in a sample, and/or how to correlate the level of one or more small non-coding RNA biomarkers in a sample with the type of cancer, tissue of origin, and status or stage of the cancer of a subject.

In some embodiments, the kit can contain one or more containers with small non-coding RNA biomarker samples, to be used as reference standards, suitable controls, or for calibration of an assay to detect the biomarkers in a test sample.

Other embodiments are described in the following non-limiting Examples. Various publications, including patents, published applications, technical articles and scholarly articles are cited throughout the specification. Each of these cited publications is incorporated by reference herein in its entirety.

### EXAMPLES

### Example 1. A Systematic Search for Orphan Small Non-Coding RNAs in Cancers

Performing a systematic screen for small non-coding RNAs that are expressed by breast cancer but are absent from healthy normal, a set of 201 small non-coding RNAs were previously identified as being strongly associated with breast cancer but mostly undetectable in normal cells. See WO 2019/094780 published on May 16, 2019 and Fish et al., Nat. Med., 2018, 24:1743-1751 (incorporated herein by reference). Because these small non-coding RNA are cancer-associated but functionally unknown, they have been named “orphan non-coding RNAs” or “oncRNAs.” The approaches and concepts were expanded and applied across several cancers to examine the cancer-specific RNA landscape.

To identify pan-cancer orphan noncoding RNAs (oncRNAs) that are expressed in cancer cells but undetectable in normal tissue, a database was designed and a systemic approach using small-RNA (smRNA) sequencing datasets from The Cancer Genome Atlas (TCGA) was implemented. 11082 datasets were downloaded via the Genomic Data Center Data Portal in BAM format. The read sequences, along with the reads' normalized counts (counts-per-million), study metadata, and chromosomal coordinates, were then uploaded onto the database and then grouped based on their strand-specific sequences across all datasets. Following the aggregation, smRNAs with low-complexity sequences were eliminated from the database. The smRNAs were then grouped based on the tissue type in which they are present, allowing for overlaps across different tissues. For each tissue type, the proportion of normal, undiseased samples and the proportion of cancer samples in which the smRNAs were present were calculated. Small-RNAs that were present in more than 10% of the normal samples for at least one tissue type or present in less than 10% of the cancer samples for every tissue type were filtered out. The remaining smRNAs were again grouped based on tissue type, allowing for overlaps. For each tissue type, a one-sided Fisher Exact Test was used to compare the presence and absence of each smRNA within the tissue group between the tissue-specific cancer samples and the normal samples of all tissue types. The smRNAs with corrected fdr<0.1 in at least one of the tissue-specific tests were selected. There were 1,543 smRNAs that satisfied all these criteria in the oncRNA database. The sequence of each of the 1,543 smRNAs thus identified are provided in TABLE 1 below together with their respective cancerous indications.

TABLE 1

| SEQ ID | | |
|---|---|---|
| NO: | Sequence | Cancers |
| 1 | CAGCTTCCATGACTCCTGATGG | acute myeloid leukemia, breast invasive |
| | | carcinoma |
| 2 | CATAGTGTAATGGTTAGCACTCTGGACT | adrenocortical carcinoma, breast invasive |
| | | carcinoma |
| 3 | AACTGATACTGGACAAGGA | breast invasive carcinoma |
| 4 | ATGCCATGATGCTAGACTCCTGAGCAGA | breast invasive carcinoma |
| 5 | GCATGGGTGGTTCAGTGGTAGAAGTCTCGC | breast invasive carcinoma |
| 6 | GTAGTCGTGGCCGAGTGTTAAGGC | breast invasive carcinoma |
| 7 | GTTATAAGCTAATTTTTTGTAAGC | breast invasive carcinoma |
| 8 | TATAAGCTAATTTTTTGTAAGC | breast invasive carcinoma |
| 9 | TGGGTCGGAAAAAAGGACTTTTT | breast invasive carcinoma, esophageal |
| | | carcinoma |
| 10 | TGGTAGAATTCACTG | breast invasive carcinoma |
| 11 | TTTGGCAATGGTAGAACTCACACTATT | breast invasive carcinoma |
| 12 | CACATGGGTCGGAAAAAAGG | adrenocortical carcinoma, colon |
| | | adenocarcinoma |
| 13 | ACATGGGTCGGAAAAAAGGA | adrenocortical carcinoma, colon |
| | | adenocarcinoma |
| 14 | AGCCTGGTCCACATGGGTCGGAA | adrenocortical carcinoma, colon |
| | | adenocarcinoma |
| 15 | CATGGGTCGGAAAAAAGG | adrenocortical carcinoma, colon |
| | | adenocarcinoma |
| 16 | GGGTCGGAAAAAAGGACTTTT | adrenocortical carcinoma, colon |
| | | adenocarcinoma |
| 17 | TGCTGTGATGACTATCTTAGGACACCT | adrenocortical carcinoma, colon |
| | | adenocarcinoma |
| 18 | TGGGTCGGAAAAAAGGACTT | adrenocortical carcinoma, colon |
| | | adenocarcinoma |
| 19 | TGGGTCGGAAAAAAGGACTTTTTT | adrenocortical carcinoma, colon |
| | | adenocarcinoma |
| 20 | AAACTGTAATTACTTTTGGACA | colon adenocarcinoma |
| 21 | AAAGGTCCCTGGTTCGTTCCCG | colon adenocarcinoma |
| 22 | AACACTGTCTGGTAAAGATGGA | colon adenocarcinoma |
| 23 | AACGCCTCGGGGACCTGTTTGA | colon adenocarcinoma |
| 24 | AAGGTCCTGAGTTCGTACCTCAGAGGG | colon adenocarcinoma |
| 25 | ACACGTGTCGGTACCGGAG | colon adenocarcinoma |
| 26 | ACAGAATCGCCGGACAGGTGGC | colon adenocarcinoma |
| 27 | ACAGACCAAGGAGAACTGGAG | colon adenocarcinoma |
| 28 | ACCGCGCCGGGCTGCGGG | colon adenocarcinoma |
| 29 | ACCTACAGAGCTCATGGTCGG | colon adenocarcinoma |
| 30 | ACCTCGTGGCGCAATGGTAGCGC | colon adenocarcinoma |
| 31 | ACTAACGGAACGTCAGGAAG | colon adenocarcinoma |
| 32 | ACTATGGAACTTTCCCCC | colon adenocarcinoma |
| 33 | ACTCGGGCTGTAGAAACTG | colon adenocarcinoma |
| 34 | ACTTGCCAGATACGGGGG | colon adenocarcinoma |
| 35 | AGACGCGACCTCAGATCAGACGTGGCGA | colon adenocarcinoma |
| 36 | AGAGCTACTGAACCAGAG | colon adenocarcinoma |
| 37 | AGCACCCCGTGCTGCTGACCGAGG | colon adenocarcinoma |
| 38 | AGCTTATCAGACTCATGTTGA | colon adenocarcinoma |
| 39 | AGGAGGTGGTTAGGAATGTGG | colon adenocarcinoma |
| 40 | AGGTACAGGCCTGGAGA | colon adenocarcinoma |
| 41 | AGTCCGCCGAGGGCGCACCAC | colon adenocarcinoma |
| 42 | AGTGGCTAAGTTCTGA | colon adenocarcinoma |
| 43 | AGTGGTTGATCGTTGG | colon adenocarcinoma |
| 44 | ATAGCTCAGTGGTAGAGCATTTGACTATCG | colon adenocarcinoma |
| 45 | ATCATTATTTGCTGCTCTA | colon adenocarcinoma |
| 46 | ATCGTAGGACAGTGAAG | colon adenocarcinoma |
| 47 | ATGAACGAGATTCCCACT | colon adenocarcinoma |
| 48 | ATGATGAAATCACCCAAAATA | colon adenocarcinoma |
| 49 | ATGGGTCGGAAAAAAGG | colon adenocarcinoma |
| 50 | ATGGTTAGCACTCTGGACTCTGAATCGT | colon adenocarcinoma |
| 51 | ATTGGTCGTGGTTGTAGGA | colon adenocarcinoma |
| 52 | ATTGTCCAGGCTCTAGAAC | colon adenocarcinoma |
| 53 | CACTGTCTGGTAAAGATGGCAT | colon adenocarcinoma |
| 54 | CAGGACTGCTCACTACTG | colon adenocarcinoma |
| 55 | CAGTGCATCACAGAACTTTGTA | colon adenocarcinoma |
| 56 | CATGGGTCAGTCGGTCCTGAGAGATCGT | colon adenocarcinoma |
| 57 | CCCAGGGTCGTGGGTTCG | colon adenocarcinoma |
| 58 | CCGCGCGCCGGCCGGGCG | colon adenocarcinoma |
| 59 | CCTCCCCGGAGGGGGCGG | colon adenocarcinoma |
| 60 | CGAGGTGGCCGAGTGGTT | colon adenocarcinoma |
| 61 | CGATCACTCCCGTTGAG | colon adenocarcinoma |
| 62 | CGATGTGATTTCTGCCCCGT | colon adenocarcinoma |
| 63 | CGCAAGACGGACCAGAGCGAAAGCA | colon adenocarcinoma |
| 64 | CGCTGTCGGTGAGTTTT | colon adenocarcinoma |
| 65 | CGGTTTGGGTCCGAGAGGTCCCG | colon adenocarcinoma |
| 66 | CGTGGATACCCTGGGA | colon adenocarcinoma |
| 67 | CTAAATGATGCAACTCTGACT | colon adenocarcinoma |
| 68 | CTCGACCGGACCGGTGC | colon adenocarcinoma |
| 69 | CTCTGAGTAAGGGGAGGAC | colon adenocarcinoma, kidney renal clear cell |
| | | carcinoma, uterine corpus endometrial |
| | | carcinoma |
| 70 | CTCTGCGTATGTGGACGGC | colon adenocarcinoma |
| 71 | CTCTGTACGGCTTCGGCGGTT | colon adenocarcinoma |
| 72 | CTGAAGAAGAGGAAACTG | colon adenocarcinoma |
| 73 | CTGAAGGAGCTGATCCAGAAG | colon adenocarcinoma |
| 74 | CTGACTTCTGAGGAAGAGGC | colon adenocarcinoma |
| 75 | CTGAGAGAACCAGACGGA | colon adenocarcinoma |
| 76 | CTGCGGGATGAACCGAA | colon adenocarcinoma |
| 77 | CTGCGGGATGAACCGAACGCCGGGTT | colon adenocarcinoma |
| 78 | CTGGGAATACCGGGTGCTGTAGGCTTCGT | colon adenocarcinoma |
| 79 | CTGTGGGATTATGACTGAACGCCTCGTATG | colon adenocarcinoma |
| 80 | CTTAATGATGACTGTTTTA | colon adenocarcinoma |
| 81 | CTTCTCACTACTGCACTTGACTAGTTCGT | colon adenocarcinoma |
| 82 | CTTCTGACACATACTTTTCT | colon adenocarcinoma |
| 83 | CTTCTGCGTCCCGCCCGCCGC | colon adenocarcinoma |
| 84 | CTTGCAGTTTGATCTCAGACTG | colon adenocarcinoma |
| 85 | CTTTCCCTAGGGCTGGATG | colon adenocarcinoma |
| 86 | CTTTTAATGGATAACAGCTAT | colon adenocarcinoma |
| 87 | GAAACCTCTGCGCCATGAGAGC | colon adenocarcinoma |
| 88 | GAAGGACTGTGGAAGTTG | colon adenocarcinoma |
| 89 | GAAGGTCGTGAGTTCGTTCCT | colon adenocarcinoma |
| 90 | GACCCAGTGGCCTAATGGATAAGGCATCGT | colon adenocarcinoma |
| 91 | GACCCTTAGCGGTGGATCACTCGGCTCGT | colon adenocarcinoma |
| 92 | GACGACCTGCTTCTGG | colon adenocarcinoma |
| 93 | GACTCTTAGCGGTGGATCACTCGGCTAGT | colon adenocarcinoma |
| 94 | GAGAGTTCAAGAGGGCGTG | colon adenocarcinoma |
| 95 | GAGGCGTGGGTTCGTATCC | colon adenocarcinoma |
| 96 | GAGGTCCCGGGTTCAGATCCC | colon adenocarcinoma |
| 97 | GAGTTCTGGGCTGTAGTGCGCCA | colon adenocarcinoma |
| 98 | GATATTCCTGGACTGACTGAT | colon adenocarcinoma |
| 99 | GATCCCGAGGCCTCTCCAGT | colon adenocarcinoma |
| 100 | GATCGAGGAGCTCACAGTCTAGT | colon adenocarcinoma |
| 101 | GATCTGGCTGCGACATCTGT | colon adenocarcinoma |
| 102 | GATGACCGTGGGACCTGCC | colon adenocarcinoma |
| 103 | GATGGACGAGAATCACGAGC | colon adenocarcinoma |
| 104 | GCCCCCCCCCCCTCGCGGC | colon adenocarcinoma |
| 105 | GCCCGGATAGCTCAGTCGGTAGAGCATCGT | colon adenocarcinoma |
| 106 | GCCCGGCTAGCTCAGTCGGTAGAGCATCGT | colon adenocarcinoma |
| 107 | GCGCGTTCGGCTGTTAACC | colon adenocarcinoma |
| 108 | GCGGAGCGAGCGCACGGGGTCG | colon adenocarcinoma |
| 109 | GCGGCGGCGGCGACTCTGGACGCGAGCCAC | colon adenocarcinoma |
| 110 | GCTGATAGACTTGAACTG | colon adenocarcinoma |
| 111 | GCTGCATGTGGCAGTCTG | colon adenocarcinoma |
| 112 | GCTTATCAGCCTGATGTTGA | colon adenocarcinoma |
| 113 | GGAAGAGCCCAGCGCCGAA | colon adenocarcinoma |
| 114 | GGAATTACAAGACAGTTG | colon adenocarcinoma |
| 115 | GGACCGAAACCCCCCCC | colon adenocarcinoma, head and neck |
| | | squamous cell carcinoma |
| 116 | GGAGATTTCAACTTAACTTGACAG | colon adenocarcinoma |
| 117 | GGAGCCATTGTGGCTCAGGCCGG | colon adenocarcinoma |
| 118 | GGAGTTGTTCCTTTGGCCAC | colon adenocarcinoma |
| 119 | GGATGAGAACTCTAAT | colon adenocarcinoma |
| 120 | GGATGGCCGAGTGGTTAAGGC | colon adenocarcinoma |
| 121 | GGCACTGGTAGAATTCACTGA | colon adenocarcinoma, lung squamous cell |
| | | carcinoma |
| 122 | GGCCGCGTGGCCTAATGGATAAGTCGTATG | colon adenocarcinoma |
| 123 | GGCTCGTTGGTCTAGGGGTATGATTCGT | colon adenocarcinoma |
| 124 | GGCTCGTTGGTCTAGGGGTATGATTTTCGT | colon adenocarcinoma |
| 125 | GGCTCGTTGGTCTAGTGGTATGATTATCGT | colon adenocarcinoma |
| 126 | GGGCAAGTCTGGTGCCAGCA | colon adenocarcinoma |
| 127 | GGGTCGGAAAAAAGGACTTT | colon adenocarcinoma |
| 128 | GGGTCGGAAAAAAGGATTTTT | colon adenocarcinoma |
| 129 | GGGTTCAGGTCCCTGTTCAG | colon adenocarcinoma |
| 130 | GGGTTCATGTCCCTGTCCAG | colon adenocarcinoma, head and neck |
| | | squamous cell carcinoma |
| 131 | GGTAGAACTCACACTG | colon adenocarcinoma |
| 132 | GGTCGGAAAAAAGGATTT | colon adenocarcinoma |
| 133 | GGTTCGGATCCCACTCCTGA | colon adenocarcinoma |
| 134 | GGTTCGGGTCCCTTC | colon adenocarcinoma |
| 135 | GGTTTGGTTCCGAGAGGTCCCG | colon adenocarcinoma |
| 136 | GTAGCAAAGTGCTTACAGTGCAG | colon adenocarcinoma |
| 137 | GTCACTAGTGGTTCCGTT | colon adenocarcinoma |
| 138 | GTCATTTCGATGGCGTGG | colon adenocarcinoma |
| 139 | GTCGGAAAAAAGGACTTTT | colon adenocarcinoma |
| 140 | GTGACTGGAAGGGCCTGA | colon adenocarcinoma |
| 141 | GTGCAAATCTATGCAAAACTGA | colon adenocarcinoma |
| 142 | GTGCTGATAACGCCAAGGTCGCG | colon adenocarcinoma |
| 143 | GTTGACCCGGGTTCGTTTC | colon adenocarcinoma |
| 144 | GTTGTGGGCTGTAGTGCGCT | colon adenocarcinoma |
| 145 | TCCCCAAATTAATACTTTTATA | colon adenocarcinoma |
| 146 | TCGTTGTAAGTTGAAAATATAG | colon adenocarcinoma |
| 147 | TCTGGGCTGTAGTGCGCTATGCCGATCGT | colon adenocarcinoma |
| 148 | TCTGTGAGAACTAGGAGC | colon adenocarcinoma, liver hepatocellular |
| | | carcinoma |
| 149 | TGAAAAGAACTTTGAAGAGAC | colon adenocarcinoma |
| 150 | TGATCAGAAAGCTGTGGA | colon adenocarcinoma |
| 151 | TGATGTAATGATTCTGC | colon adenocarcinoma |
| 152 | TGCAGATCTTGGTGGTAGTAGCAAATCGT | colon adenocarcinoma |
| 153 | TGCATCACAGAACTTTGTT | colon adenocarcinoma |
| 154 | TGCTGATCGGCTGTATG | colon adenocarcinoma |
| 155 | TGGAAGATCGAAAGACTG | colon adenocarcinoma |
| 156 | TGGAATGGAAATCCATTGGG | colon adenocarcinoma |
| 157 | TGGATTGGAAATCCATTG | colon adenocarcinoma, kidney renal clear cell |
| | | carcinoma |
| 158 | TGGATTTGAAATCCATTGGG | colon adenocarcinoma |
| 159 | TGGCACTGGCAGAATTCACTG | colon adenocarcinoma |
| 160 | TGGCGCAATGAAGGTGAAGG | colon adenocarcinoma |
| 161 | TGGGCTGTAGTGCGCTATGCCGATCGTATG | colon adenocarcinoma |
| 162 | TGGGTCGGAAAAAAGG | colon adenocarcinoma |
| 163 | TGGGTCGGAAAAAAGGA | colon adenocarcinoma |
| 164 | TGTAGTTTCCTGTTGTTGG | colon adenocarcinoma |
| 165 | TGTCATCTTCCTCATCGGTATCATCGTATG | colon adenocarcinoma |
| 166 | TGTTAGTGATGATTTTAAAA | colon adenocarcinoma |
| 167 | TTAGATCGCTGTGAAGGA | colon adenocarcinoma |
| 168 | TTCCAGGTTCGTCTCCTG | colon adenocarcinoma |
| 169 | TTCTGTGGAGCTGTGAAGGA | colon adenocarcinoma |
| 170 | TTGACCTCTGAATTGACAGCC | colon adenocarcinoma |
| 171 | TTGCTGCTGTGCAGAAG | colon adenocarcinoma |
| 172 | TTGGCAATGGTAGAACTCCCAC | colon adenocarcinoma |
| 173 | AGATCGGAACTGGTAGACT | cervical squamous cell carcinoma and |
| | | endocervical adenocarcinoma, liver |
| | | hepatocellular carcinoma |
| 174 | AACTGGCCCTCAAAGTCCCGATT | liver hepatocellular carcinoma |
| 175 | AATGATGTTGATCAAATGTCTG | liver hepatocellular carcinoma |
| 176 | AATGGTGAGCACTTTGGACTCTGT | liver hepatocellular carcinoma |
| 177 | ACTGTGATGAACTTCTGAGG | liver hepatocellular carcinoma, uterine corpus |
| | | endometrial carcinoma |
| 178 | AGCCATGATGATACCACTGAG | liver hepatocellular carcinoma, uterine corpus |
| | | endometrial carcinoma |
| 179 | AGGAGGACTGGGACCCATGAA | liver hepatocellular carcinoma |
| 180 | AGGTGACTGGTTAATAGAACTA | liver hepatocellular carcinoma |
| 181 | ATGGACTGAGCCATGCTACT | liver hepatocellular carcinoma |
| 182 | ATGTCTGACCTGAAATGAGCATGTAGACA | liver hepatocellular carcinoma |
| 183 | ATTAATGATGAGATATAACCGTGACTGAAG | liver hepatocellular carcinoma |
| 184 | ATTTCTGACGAGATCTTGGATGATGC | liver hepatocellular carcinoma |
| 185 | CAAAAGTAACTGTGGTTTTG | liver hepatocellular carcinoma |
| 186 | CAACACCAGTCGATGGGCTAAC | liver hepatocellular carcinoma, uterine corpus |
| | | endometrial carcinoma |
| 187 | CAACACCTGTCGATGGGCTGTCA | liver hepatocellular carcinoma |
| 188 | CAAGTCACTAGTGGTTCCGTGTAG | liver hepatocellular carcinoma |
| 189 | CACAGTGATGAACAAGACAGACCTGATG | liver hepatocellular carcinoma, uveal melanoma |
| 190 | CCAGATCGTCTGTGAACTCAGG | liver hepatocellular carcinoma |
| 191 | CCCCCACTGCTAAATTTGACGGG | liver hepatocellular carcinoma |
| 192 | CCTGGGTGATGATAAGCAAATGCTGACT | liver hepatocellular carcinoma |
| 193 | CGGGACTGAGCCATGCTAC | liver hepatocellular carcinoma |
| 194 | CGTGATGATGACTTGCTGAAC | liver hepatocellular carcinoma |
| 195 | CTAACCGATGAATTGACAGC | liver hepatocellular carcinoma |
| 196 | CTAGAGGAGCCTGTTCTGTACA | liver hepatocellular carcinoma |
| 197 | CTCACCTATGAATTGAC | liver hepatocellular carcinoma |
| 198 | CTGCAAAGGGAAGCCCTTTC | liver hepatocellular carcinoma |
| 199 | CTGCACTGAGCCATGCTACT | liver hepatocellular carcinoma |
| 200 | CTGGACTGAGCCATGCTAAGA | liver hepatocellular carcinoma |
| 201 | CTGGACTGAGCCATGCTACTGAC | liver hepatocellular carcinoma |
| 202 | CTGGACTGAGCCATGCTACTGAG | liver hepatocellular carcinoma |
| 203 | CTGGACTGAGCCATGCTACTGGAC | liver hepatocellular carcinoma |
| 204 | CTGGACTGAGCCATGCTACTGGAG | liver hepatocellular carcinoma |
| 205 | CTGGACTGAGCCATGCTACTGGAGA | liver hepatocellular carcinoma |
| 206 | CTGGACTGAGCCATGCTACTGGTTT | liver hepatocellular carcinoma |
| 207 | CTGGACTGAGCCATGCTTT | liver hepatocellular carcinoma |
| 208 | CTGGACTGAGTCATGCTACTGG | liver hepatocellular carcinoma |
| 209 | CTGGATGAGGATAAGCAAATGCTGACTGA | liver hepatocellular carcinoma |
| 210 | CTGGATGATGATAAGCAAATGCCGACTGA | liver hepatocellular carcinoma |
| 211 | CTGGATGATGATAAGCAAATGCTGACGGA | liver hepatocellular carcinoma, uterine |
| | | carcinosarcoma |
| 212 | CTGGATGATGTAGCCTGACA | liver hepatocellular carcinoma |
| 213 | CTTCGTGATCGATGTGGTGACGTCGA | liver hepatocellular carcinoma |
| 214 | CTTTAGCTCTAGAATTACTCTG | liver hepatocellular carcinoma |
| 215 | CTTTGTGACCTCTGATG | liver hepatocellular carcinoma |
| 216 | GAATATGATGGTTGCTGAGA | liver hepatocellular carcinoma |
| 217 | GACAATGATGAACTGCTGA | liver hepatocellular carcinoma |
| 218 | GATTATGATGAATTCTGAAA | liver hepatocellular carcinoma |
| 219 | GCGAAGGTTGTCGGGATC | liver hepatocellular carcinoma |
| 220 | GGAGGGCCGGCGGCGGCGGCGACT | liver hepatocellular carcinoma |
| 221 | GTAGCTTATCAGACTGTTGTTGA | liver hepatocellular carcinoma |
| 222 | GTCACTAGTGGTTCCGTTTAGT | liver hepatocellular carcinoma |
| 223 | GTCCCGGGTTCAGATCCCGGACG | liver hepatocellular carcinoma |
| 224 | GTGCGCGCGGGTCGGGGCGGCGGCG | liver hepatocellular carcinoma |
| 225 | GTGGACTGAGCCGTGCTACTGA | liver hepatocellular carcinoma |
| 226 | NTGGACTGAGCCATGCTACTGG | liver hepatocellular carcinoma |
| 227 | TAATTGAAGTTCTTGCCATCAC | liver hepatocellular carcinoma |
| 228 | TCCCACTCCTGACACCATTT | liver hepatocellular carcinoma |
| 229 | TCCGGCTCGGAGGACCATAG | liver hepatocellular carcinoma, uterine corpus |
| | | endometrial carcinoma |
| 230 | TCTTTGTGACCTCTGATG | liver hepatocellular carcinoma |
| 231 | TGACCTATTAATTGACAGCCAAT | liver hepatocellular carcinoma |
| 232 | TGCCGGTGATGCTGATCTGAGG | liver hepatocellular carcinoma |
| 233 | TTACGGCTCGAAGGACCAAG | liver hepatocellular carcinoma |
| 234 | TTCTGACGAGATCTTGGATGATGC | liver hepatocellular carcinoma |
| 235 | TTCTGACGAGATCTTGGATGATGCT | liver hepatocellular carcinoma |
| 236 | TTGGACTGAGCCATGCTACTGG | liver hepatocellular carcinoma |
| 237 | TTTCTGACGAGATCTTGGATGATGCT | liver hepatocellular carcinoma |
| 238 | TTTGGCGATGGTAGAACTCACACTGT | liver hepatocellular carcinoma |
| 239 | TTTTCTGACATGCTCTAAAGGATGATGA | liver hepatocellular carcinoma |
| 240 | TTTTGTTATCTAGCTGTATG | liver hepatocellular carcinoma |
| 241 | AACTGGATTTTTGGAGCAGG | adrenocortical carcinoma, lung adenocarcinoma |
| 242 | AAGAGCCCAGCGCCGAATCCCCGCC | adrenocortical carcinoma, esophageal |
| | | carcinoma , lung adenocarcinoma |
| 243 | AATTGATTTTTGGAGCAGGGAGAT | adrenocortical carcinoma, lung adenocarcinoma |
| 244 | AGTGGATTTTTGGAGCAGG | adrenocortical carcinoma, lung adenocarcinoma |
| 245 | ATGTATTTTTGGAGCAGGGAGA | adrenocortical carcinoma, lung adenocarcinoma |
| 246 | ATGTATTTTTGGAGCAGGGAGAT | adrenocortical carcinoma, cervical squamous |
| | | cell carcinoma and endocervical |
| | | adenocarcinoma, lung adenocarcinoma |
| 247 | ATTGGATTTTTGGAGCAGGGA | adrenocortical carcinoma, cervical squamous |
| | | cell carcinoma and endocervical |
| | | adenocarcinoma, lung adenocarcinoma |
| 248 | CATTTTGGAACAATGTAGGTAA | adrenocortical carcinoma, lung squamous cell |
| | | carcinoma |
| 249 | GATTTTGGCCTAGTTCTGTG | adrenocortical carcinoma, lung squamous cell |
| | | carcinoma |
| 250 | GCCGCGAGGCGTCCAGTGCGGTAACGCGA | adrenocortical carcinoma, lung adenocarcinoma |
| 251 | GTCTCTGGAGCGTCGGGCCCA | adrenocortical carcinoma, lung squamous cell |
| | | carcinoma |
| 252 | TGTGCGGACCAGGGGAATC | bladder urothelial carcinoma, lung |
| | | adenocarcinoma |
| 253 | AATTCCGATAACGAACGCGACTCTG | cervical squamous cell carcinoma and |
| | | endocervical adenocarcinoma, lung |
| | | adenocarcinoma |
| 254 | ACGCGCATGAATGGATGCACG | cervical squamous cell carcinoma and |
| | | endocervical adenocarcinoma, lung |
| | | adenocarcinoma |
| 255 | AGACTGTGATGACTGGGAGAG | cervical squamous cell carcinoma and |
| | | endocervical adenocarcinoma, lung squamous |
| | | cell carcinoma |
| 256 | CTCTAACTTTGAAGGCCGAAG | cervical squamous cell carcinoma and |
| | | endocervical adenocarcinoma, lung squamous |
| | | cell carcinoma |
| 257 | GACAGGGAGTCGGGTTCAGAT | cervical squamous cell carcinoma and |
| | | endocervical adenocarcinoma, lung squamous |
| | | cell carcinoma |
| 258 | ATGTGAATCCGACTGTT | cervical squamous cell carcinoma and |
| | | endocervical adenocarcinoma, lung squamous |
| | | cell carcinoma |
| 259 | CATGGTGACCACGGGTGCCG | cervical squamous cell carcinoma and |
| | | endocervical adenocarcinoma, lung |
| | | adenocarcinoma |
| 260 | CGCCTGTCACGCGGGAGCCCGG | cervical squamous cell carcinoma and |
| | | endocervical adenocarcinoma, lung |
| | | adenocarcinoma |
| 261 | GCGCCGCCGCCCCCCCCCCG | cervical squamous cell carcinoma and |
| | | endocervical adenocarcinoma, lung |
| | | adenocarcinoma, pancreatic adenocarcinoma |
| 262 | GCGCGGACCAGGGGAATCC | cervical squamous cell carcinoma and |
| | | endocervical adenocarcinoma, lung squamous |
| | | cell carcinoma |
| 263 | GCTACCACATCCAAGGACGG | cervical squamous cell carcinoma and |
| | | endocervical adenocarcinoma, lung |
| | | adenocarcinoma |
| 264 | GGACCAGCCCGTGGACGGTGTG | cervical squamous cell carcinoma and |
| | | endocervical adenocarcinoma, head and neck |
| | | squamous cell carcinoma, lung adenocarcinoma, |
| | | pancreatic adenocarcinoma |
| 265 | GGCCGCCGCCGGGCGCA | cervical squamous cell carcinoma and |
| | | endocervical adenocarcinoma, lung squamous |
| | | cell carcinoma, skin cutaneous melanoma |
| 266 | GTCGGGGTTTCGTACGTCG | cervical squamous cell carcinoma and |
| | | endocervical adenocarcinoma, lung |
| | | adenocarcinoma |
| 267 | TGGGATCCCGAGGCCTCTCCAG | cervical squamous cell carcinoma and |
| | | endocervical adenocarcinoma, lung |
| | | adenocarcinoma |
| 268 | CCCGTGCCGAGTCGTGACCGGTG | esophageal carcinoma , lung squamous cell |
| | | carcinoma |
| 269 | AACAGGGTTTGTTAGG | head and neck squamous cell carcinoma, lung |
| | | adenocarcinoma |
| 270 | AAGCGGAGTCGGGTTCAGA | head and neck squamous cell carcinoma, lung |
| | | adenocarcinoma |
| 271 | ACGAAAGCCGCCGTGGCG | head and neck squamous cell carcinoma, lung |
| | | adenocarcinoma |
| 272 | AGAGGCAACTCTGGTGGAGG | head and neck squamous cell carcinoma, lung |
| | | squamous cell carcinoma |
| 273 | AGCTGTTGAACATGGGTCAGTCGG | head and neck squamous cell carcinoma, lung |
| | | adenocarcinoma |
| 274 | ATGGCCAGAGCTCACACAGAGG | head and neck squamous cell carcinoma, lung |
| | | adenocarcinoma |
| 275 | CAACACCCGTCGATGGGCT | head and neck squamous cell carcinoma, lung |
| | | adenocarcinoma |
| 276 | CAGGTGTCCTAAGGCGAGCTCA | head and neck squamous cell carcinoma, lung |
| | | adenocarcinoma |
| 277 | CAGTCCGCCGAGGGCGCACC | head and neck squamous cell carcinoma, lung |
| | | squamous cell carcinoma |
| 278 | CCGGGTGATGTAGGCTT | head and neck squamous cell carcinoma, lung |
| | | adenocarcinoma |
| 279 | GAGCCAGTCGGTCCTGAGAG | head and neck squamous cell carcinoma, lung |
| | | squamous cell carcinoma |
| 280 | GCCAAAGCTCGCTTGATCTT | head and neck squamous cell carcinoma, lung |
| | | squamous cell carcinoma |
| 281 | GTGCTGTGATGAGCTCTGAGC | kidney renal clear cell carcinoma, lung |
| | | adenocarcinoma |
| 282 | TGTAGGTAAGGGAAGTCGGCAAGCCG | kidney renal clear cell carcinoma, lung |
| | | adenocarcinoma |
| 283 | GTGCTGTGATGAGCTCTGAGCC | head and neck squamous cell carcinoma, kidney |
| | | renal clear cell carcinoma, lung adenocarcinoma |
| 284 | AAAATTCAACGCTGTCGGTTAG | lung adenocarcinoma |
| 285 | AAACGGATTTTTGGAGCAGG | lung adenocarcinoma |
| 286 | AACACCAGTCGATGGGCTGTAAG | lung adenocarcinoma |
| 287 | AACGATGCCGACCGGCGCTGCGG | lung adenocarcinoma |
| 288 | AACTAGACTCTGGCA | lung adenocarcinoma |
| 289 | AAGCATTTTTGGAAATAGGAGA | lung adenocarcinoma |
| 290 | AATAGCTCAGAATGTCAGT | lung adenocarcinoma |
| 291 | ACAGCACTAGATTGTAAAGACTGGGGTGG | lung adenocarcinoma |
| 292 | ACCCGGTCAGCCCCTCT | lung adenocarcinoma |
| 293 | ACCGGGTGCTGTCGGCTTG | lung adenocarcinoma |
| 294 | AGATCTGGGCTGTAGTGCG | lung adenocarcinoma |
| 295 | AGCACTAGATTGTAAAGACTGGGGTG | lung adenocarcinoma |
| 296 | AGTAGGAGGGCCGCTGCGGTGAG | lung adenocarcinoma |
| 297 | ATCCCACCTTCGTCGCCAT | lung adenocarcinoma, uterine corpus |
| | | endometrial carcinoma |
| 298 | ATCGATTTTTGGAGCAGGG | lung adenocarcinoma |
| 299 | ATGCATTTTTGGAGCAGGGA | lung adenocarcinoma |
| 300 | ATGCTGACTGAACATGAAGGTC | lung adenocarcinoma |
| 301 | ATGGATTTTTGGAAATCGGA | lung adenocarcinoma |
| 302 | ATGGATTTTTGGCGCAGGGA | lung adenocarcinoma |
| 303 | ATTAGCAATGGTAGAACTCAC | lung adenocarcinoma |
| 304 | CAAACCGGGTGCTGTAGGCT | lung adenocarcinoma |
| 305 | CAACACCAGTAGATGG | lung adenocarcinoma |
| 306 | CAAGACGGACCAGAGCG | lung adenocarcinoma |
| 307 | CACAGCTGGGTTGAGAGGGCGT | lung adenocarcinoma |
| 308 | CACGACGGTGGCCATGGAAGTCGGA | lung adenocarcinoma |
| 309 | CAGCCAGTAGTGGACAGG | lung adenocarcinoma |
| 310 | CAGCGCGGTAACGCGACCGATCCCGGAGA | lung adenocarcinoma |
| 311 | CAGGATCGCGTGAGCTCGGAG | lung adenocarcinoma |
| 312 | CATCTCGCTGTAGCATCGAACC | lung adenocarcinoma |
| 313 | CATGGTGCTGTCGGAGCTAG | lung adenocarcinoma |
| 314 | CCACCGCCCGTCCCCG | lung adenocarcinoma |
| 315 | CCCCCCACAACCGCGCTTGACTGG | lung adenocarcinoma, pancreatic |
| | | adenocarcinoma |
| 316 | CCCCGAGGGGCTCTCGCTTCTGGCG | lung adenocarcinoma |
| 317 | CCTACCAGAGTCGCCA | lung adenocarcinoma |
| 318 | CCTATTGGCCAGTTTTGTCTGATG | lung adenocarcinoma |
| 319 | CCTCGGGCCGCTCGCAC | lung adenocarcinoma |
| 320 | CCTGAGGGAGCTCGTCGGTG | lung adenocarcinoma |
| 321 | CGAGCTTATCAGACTGATGTTGAC | lung adenocarcinoma |
| 322 | CGAGGCCCAGCCCGTGGCCGGTGTGA | lung adenocarcinoma |
| 323 | CGGCGACGGGGGGGGTGCCGCG | lung adenocarcinoma |
| 324 | CGGCGCCGCTCGTGGGGG | lung adenocarcinoma |
| 325 | CGTACATTGCCAGGGATTTC | lung adenocarcinoma |
| 326 | CGTGTGGTGTGCGTCGGCGGGC | lung adenocarcinoma |
| 327 | CTAGCACTGGTAGAATTCA | lung adenocarcinoma |
| 328 | CTCCCGTGCTGATCAGTCGTGG | lung adenocarcinoma |
| 329 | CTCTCTCTCTCTGCCT | lung adenocarcinoma |
| 330 | CTGGAATTCTGGACCCGG | lung adenocarcinoma |
| 331 | CTGTGCGTGCGACAGCGGCTG | lung adenocarcinoma |
| 332 | CTGTGCGTGTGACAGCGGCTTC | lung adenocarcinoma |
| 333 | CTTGTGGTGTGCGTCGGAGGG | lung adenocarcinoma |
| 334 | GAATGTTTAGACGGGCTCTGG | lung adenocarcinoma |
| 335 | GACACCAGGCCGGCCCCGG | lung adenocarcinoma |
| 336 | GACGAATTTTTGAGCGGGTA | lung adenocarcinoma |
| 337 | GAGCACAGCATCGTCGGG | lung adenocarcinoma |
| 338 | GAGCCCAGGAACTGGAGGCTGT | lung adenocarcinoma |
| 339 | GAGCCGAAGCTGGACTGTACTG | lung adenocarcinoma |
| 340 | GAGGCCAGCGCCGAATCCCCG | lung adenocarcinoma |
| 341 | GCCCCCCGTGGCGGCGACGACCCAT | lung adenocarcinoma |
| 342 | GCCCCGAGGCGTCCAGTGCGG | lung adenocarcinoma |
| 343 | GCCGCCGGTGCAATACCACTACTCTG | lung adenocarcinoma |
| 344 | GCGACGAGTAGGAGGGCCGCTG | lung adenocarcinoma |
| 345 | GCGCTCGCCGGCCGAGGTGGGAT | lung adenocarcinoma |
| 346 | GGCGGCGGCGGCAGGCGGCG | lung adenocarcinoma |
| 347 | GGGATGAACCGACCGCCGG | lung adenocarcinoma |
| 348 | GGGCTGTGATGAAGCTGAGCA | lung adenocarcinoma |
| 349 | GGTCCCGTGCCCGTCGTCGTC | lung adenocarcinoma, skin cutaneous |
| | | melanoma |
| 350 | GTAGCTTATCAGCCTGATGTT | lung adenocarcinoma |
| 351 | GTGCGGAACGCTGGCCGG | lung adenocarcinoma |
| 352 | GTGCGGTAACGCGACCGCTCCCGG | lung adenocarcinoma |
| 353 | TAATGTGATGAACTTCTGAGG | lung adenocarcinoma, mesothelioma |
| 354 | TACTTTCTCAGACTGATGTTGA | lung adenocarcinoma |
| 355 | TAGATGTCCGGGGCTGCCCG | lung adenocarcinoma |
| 356 | TATCGCACTGGTAGAATTCAAT | lung adenocarcinoma |
| 357 | TATCGCACTTGTAGAATTCACT | lung adenocarcinoma |
| 358 | TATCGCGCTGGTAGAATTCACTG | lung adenocarcinoma |
| 359 | TATGATTCTCGCTTCGGTGTG | lung adenocarcinoma |
| 360 | TATGTTCGTTCGGCTCGCGTGAAG | lung adenocarcinoma |
| 361 | TATTGCACTCGTCCCGA | lung adenocarcinoma |
| 362 | TATTGTACTGGTAGAATTCACTG | lung adenocarcinoma |
| 363 | TCACACCAGAGTCGCCA | lung adenocarcinoma |
| 364 | TCAGGTCTCGGTGGAACCTC | lung adenocarcinoma |
| 365 | TCATCATCGGACTGGACGGGAGCTT | lung adenocarcinoma |
| 366 | TCTCACTGTAGCATCGACC | lung adenocarcinoma |
| 367 | TCTGGACTCTGAATCCGGTA | lung adenocarcinoma |
| 368 | TGAGGCAATGCTAGAACTCACA | lung adenocarcinoma |
| 369 | TGCTATGATGAACTGTCCTGAGA | lung adenocarcinoma |
| 370 | TGGTCGTGGTTGTAGTCCCCA | lung adenocarcinoma |
| 371 | TGTTGCACTCGTCCCGGCC | lung adenocarcinoma |
| 372 | TTACAGCCCCCCCGGCA | lung adenocarcinoma |
| 373 | TTCATCTTAAACTGAAC | lung adenocarcinoma |
| 374 | TTCCTTATCACACTGATGTTGAC | lung adenocarcinoma |
| 375 | TTCTTTATAAGACTGATGTTGAC | lung adenocarcinoma |
| 376 | TTGAAGGCTCCGCGGACCGG | lung adenocarcinoma |
| 377 | TTGCTTATCAGACTGAAGTTGAC | lung adenocarcinoma |
| 378 | TTGGATTTTTGGAAATAGG | lung adenocarcinoma |
| 379 | TTGGTGATCAGACTGATGTTG | lung adenocarcinoma |
| 380 | TTGGTTATCAGACTGAAGTTGAC | lung adenocarcinoma |
| 381 | TTGGTTATCAGACTGATGATGA | lung adenocarcinoma |
| 382 | TTGGTTATCAGACTGATGCTG | lung adenocarcinoma |
| 383 | TTTAGCAATGGTAGAACTCAAAC | lung adenocarcinoma |
| 384 | TTTGGCAATGGTAGAATTAAC | lung adenocarcinoma |
| 385 | TTTTGCAATGGTAGAAATAACACT | lung adenocarcinoma |
| 386 | AAATCACTGGTAGAATTCACTG | lung squamous cell carcinoma |
| 387 | AAATGAGGCCATGATTAAGAGG | lung squamous cell carcinoma |
| 388 | AACCCCATTCGTGATGG | lung squamous cell carcinoma |
| 389 | AACGTCGGGCCGATCGCA | lung squamous cell carcinoma |
| 390 | AACTCGGAGGTTCGAAGACG | lung squamous cell carcinoma |
| 391 | AAGACACTGGTAGAATTCAC | lung squamous cell carcinoma |
| 392 | AATCAGCGGGGAAAGAA | lung squamous cell carcinoma |
| 393 | AATGGATTTTTGGAAATAGGG | lung squamous cell carcinoma |
| 394 | AATGTATTGTACATCGGATGAG | lung squamous cell carcinoma |
| 395 | ACAGCAGATCAGACGTGGCGACC | lung squamous cell carcinoma |
| 396 | ACAGGGTGCTGTAGGATT | lung squamous cell carcinoma |
| 397 | ACCCGACCTCAGATCAGAC | lung squamous cell carcinoma |
| 398 | ACCGAAGTCTTTGGGTTCCGG | lung squamous cell carcinoma |
| 399 | ACGCGTTAGGACCCGAAA | lung squamous cell carcinoma |
| 400 | ACGTGGCGACCCGCTGAATTT | lung squamous cell carcinoma |
| 401 | ACGTTCGTGGGGAACC | lung squamous cell carcinoma |
| 402 | ACTCCCTGATGAACA | lung squamous cell carcinoma |
| 403 | ACTGCGGGATAAGGATTGGCTCTA | lung squamous cell carcinoma |
| 404 | AGACGACTTAGAACTGGTG | lung squamous cell carcinoma |
| 405 | AGAGATGTGGTGACGTCGTG | lung squamous cell carcinoma |
| 406 | AGCGCACTGGTAGAATTCAC | lung squamous cell carcinoma |
| 407 | AGCTGGTGCTGTAGGCT | lung squamous cell carcinoma |
| 408 | AGCTGTCCGGGGCTGCACG | lung squamous cell carcinoma |
| 409 | AGGAACCGCAGGTTCAGACA | lung squamous cell carcinoma |
| 410 | AGGACAGAAACCTCCCGTGGAGCA | lung squamous cell carcinoma |
| 411 | AGGCCGCGGAGCCAGCGACGAC | lung squamous cell carcinoma, testicular germ |
| | | cell tumors, uterine corpus endometrial |
| | | carcinoma |
| 412 | AGGTATTTTTGGAGCAGGGAGA | lung squamous cell carcinoma |
| 413 | AGTCGGGGGCTCGCA | lung squamous cell carcinoma |
| 414 | ATCCCACTACTCTGATCGT | lung squamous cell carcinoma |
| 415 | ATCCCAGCGCCGAATCCCC | lung squamous cell carcinoma |
| 416 | ATCCCCGGCCGTCGCCGGC | lung squamous cell carcinoma |
| 417 | ATCTCAAAAGCTCGCTTGATCT | lung squamous cell carcinoma |
| 418 | ATGCCAAAAGCTCGCTTGATC | lung squamous cell carcinoma |
| 419 | ATGTCAGTGGTAGAATTCACT | lung squamous cell carcinoma |
| 420 | ATGTGTGGTTCAGTGGTAGA | lung squamous cell carcinoma |
| 421 | CAACACCAGTCGATGGCCTG | lung squamous cell carcinoma |
| 422 | CAACATGATGAAACCCTGACT | lung squamous cell carcinoma |
| 423 | CACATGCTCAGACTCCTGTGGT | lung squamous cell carcinoma |
| 424 | CACCGCGGCGGTGCG | lung squamous cell carcinoma |
| 425 | CACGCACCGCACGTTCGTG | lung squamous cell carcinoma |
| 426 | CACTGACCAAGGAGTCTAAC | lung squamous cell carcinoma |
| 427 | CAGACCAGGGGAATCCGACTG | lung squamous cell carcinoma |
| 428 | CAGAGCACAGCATCGTCGGG | lung squamous cell carcinoma |
| 429 | CAGCCCAGGGAACGGGCTTG | lung squamous cell carcinoma |
| 430 | CAGCCGGTCCTGAGAGATGGG | lung squamous cell carcinoma |
| 431 | CATGTGTTAGGACCCGAAAG | lung squamous cell carcinoma |
| 432 | CCACGCCCGGGGCACCCC | lung squamous cell carcinoma |
| 433 | CCAGCCACTGCTAAATTTGAC | lung squamous cell carcinoma |
| 434 | CCAGGACACGGACAGGATTG | lung squamous cell carcinoma |
| 435 | CCAGGACACGGACAGGATTG | lung squamous cell carcinoma |
| 436 | CCAGGGAGCGGGGGACCACCAG | lung squamous cell carcinoma |
| 437 | CCAGTCCGCCGAGGGCGCAC | lung squamous cell carcinoma |
| 438 | CCATACCCCCCCGGCCCCGTC | lung squamous cell carcinoma |
| 439 | CCCACTGCTAAATTTGCCTGG | lung squamous cell carcinoma |
| 440 | CCCCCAGGAGTGGAGCCTGCG | lung squamous cell carcinoma |
| 441 | CCCCTACTCTGATCGTTTTTT | lung squamous cell carcinoma |
| 442 | CCCGACCCGGGGAGGTAGTGAC | lung squamous cell carcinoma |
| 443 | CCCGCACTGCTAAATTTGAC | lung squamous cell carcinoma |
| 444 | CCCTACCTACTATCC | lung squamous cell carcinoma |
| 445 | CCGAAGTCTTTGGGTTCCGG | lung squamous cell carcinoma |
| 446 | CCGCAGGTGCAGATCTTGGTG | lung squamous cell carcinoma |
| 447 | CCGCGCGGCGCCTCGCCTCGGCCGGCGCCT | lung squamous cell carcinoma |
| 448 | CCGCGGAGCCTCGGTT | lung squamous cell carcinoma |
| 449 | CCGGCCCCACCGGTCCCGG | lung squamous cell carcinoma |
| 450 | CCGGCGGGCCCGGGGGAGG | lung squamous cell carcinoma |
| 451 | CCGTAGAGGGAGCCTGAGA | lung squamous cell carcinoma |
| 452 | CCGTCCCCCTAAGCGCAGACCC | lung squamous cell carcinoma |
| 453 | CCTTTACGACTCTTAGCGGTG | lung squamous cell carcinoma |
| 454 | CGAGGCGTGTGACAGCGGCT | lung squamous cell carcinoma |
| 455 | CGAGGGAGCGAGACCCG | lung squamous cell carcinoma |
| 456 | CGCGCAAATTACCCACTCCCGAC | lung squamous cell carcinoma |
| 457 | CGCGCGTGTGGTGTG | lung squamous cell carcinoma |
| 458 | CGCTCTGAGCGTCGCT | lung squamous cell carcinoma |
| 459 | CGGCGGTGCGCCGCGACCGGCTCCGGGAC | lung squamous cell carcinoma |
| 460 | CGGGTGCTGTAGGCTCTG | lung squamous cell carcinoma |
| 461 | CGTGGGTTATCTAGCTGTATG | lung squamous cell carcinoma |
| 462 | CGTTCCCGGGTTTCGGCAGA | lung squamous cell carcinoma |
| 463 | CTAAGGGCTGGGTCGGGCGGGC | lung squamous cell carcinoma |
| 464 | CTAGACCTCAGATCAGACGTGGCGACCC | lung squamous cell carcinoma |
| 465 | CTCAAGGGAGTCGGGTTCAG | lung squamous cell carcinoma |
| 466 | CTCAAGTCGGAGGTTCGAAGA | lung squamous cell carcinoma |
| 467 | CTCAGTTGGATCTTGGGAGCG | lung squamous cell carcinoma |
| 468 | CTCCACCAGTCGATGGGCTG | lung squamous cell carcinoma |
| 469 | CTCCCATGGTGACCACG | lung squamous cell carcinoma |
| 470 | CTCCTACCGATTGGATGG | lung squamous cell carcinoma |
| 471 | CTCCTTAGAACTGGTGCG | lung squamous cell carcinoma |
| 472 | CTCGATAACCTCGGGCCGA | lung squamous cell carcinoma |
| 473 | CTCGATAACCTCGGGCCGATCG | lung squamous cell carcinoma |
| 474 | CTCGCTGGCCCTTGAAAAT | lung squamous cell carcinoma |
| 475 | CTCGGTTTCTATCTCTCCATCC | lung squamous cell carcinoma |
| 476 | CTCTCCCCCGCTCCCCGCC | lung squamous cell carcinoma |
| 477 | CTCTCTGCCTGTTTCTCTTT | lung squamous cell carcinoma |
| 478 | CTGCGGGATGAACCGA | lung squamous cell carcinoma |
| 479 | CTGGGAAAGAAGACCCTGTTG | lung squamous cell carcinoma |
| 480 | CTGGGATTATGACTGAACG | lung squamous cell carcinoma |
| 481 | CTGTACACAAGGGTTTGT | lung squamous cell carcinoma |
| 482 | CTGTAGCGTCGGGCCCAT | lung squamous cell carcinoma |
| 483 | CTGTGCGTGTGACAGCGCC | lung squamous cell carcinoma |
| 484 | CTGTGCTGTAGTGCGCTATGCCG | lung squamous cell carcinoma |
| 485 | CTGTGCTTGTGACAGCGGCTGT | lung squamous cell carcinoma |
| 486 | CTGTGTGTGTGACAGCGGCTG | lung squamous cell carcinoma |
| 487 | CTGTTGCTGTCGGCTT | lung squamous cell carcinoma |
| 488 | CTTCTGGCGCCAAGCGCCC | lung squamous cell carcinoma |
| 489 | CTTTGGAGCGGGCGGGCGGTCC | lung squamous cell carcinoma |
| 490 | GAACCATCTAGTAGCT | lung squamous cell carcinoma |
| 491 | GACAAGAACTTTGAAGAG | lung squamous cell carcinoma |
| 492 | GACCGCACTTGCGGCCCCG | lung squamous cell carcinoma |
| 493 | GACCGCCGGTGAAATACCACTACTCTGA | lung squamous cell carcinoma |
| 494 | GACCTGGTGCGGACCAGGGGAA | lung squamous cell carcinoma, pancreatic |
| | | adenocarcinoma |
| 495 | GACGCTTAGCGGTGGAT | lung squamous cell carcinoma |
| 496 | GACGGAAGGGCACCACC | lung squamous cell carcinoma |
| 497 | GAGAGTTCAAGAGGGCGTGAA | lung squamous cell carcinoma |
| 498 | GAGCGAAAGCATTTGCCAA | lung squamous cell carcinoma |
| 499 | GATGCCGACGCTCATCAGAC | lung squamous cell carcinoma |
| 500 | GATGGGCTCTAAGGGCTGG | lung squamous cell carcinoma |
| 501 | GATTTCCGGGGCTGCACGC | lung squamous cell carcinoma |
| 502 | GATTTGTTGTTGCCATGG | lung squamous cell carcinoma |
| 503 | GCACTGGAGCGTCGGGCC | lung squamous cell carcinoma |
| 504 | GCAGACTTAGAACTGGTG | lung squamous cell carcinoma |
| 505 | GCATACTTAGAACTGGTGCGGACC | lung squamous cell carcinoma |
| 506 | GCATCGAGGCGTCCAGTGCGG | lung squamous cell carcinoma |
| 507 | GCATGATGATGAGCACTCTGAAG | lung squamous cell carcinoma |
| 508 | GCCATGGTAATCCTGCT | lung squamous cell carcinoma |
| 509 | GCCCCCCGTCCTCCCCCCT | lung squamous cell carcinoma |
| 510 | GCCCGGAGGATTCAACCC | lung squamous cell carcinoma |
| 511 | GCCGCGGCCGGGGGGCGGGCGC | lung squamous cell carcinoma |
| 512 | GCCTACTTAGAACTG | lung squamous cell carcinoma |
| 513 | GCCTGGCGTGGAATGCG | lung squamous cell carcinoma |
| 514 | GCGCACCAGGGGAATCC | lung squamous cell carcinoma |
| 515 | GCGCGTCCCCCGAAGA | lung squamous cell carcinoma |
| 516 | GCGGGCATTTATCAGATC | lung squamous cell carcinoma |
| 517 | GCGGTGAGCCTTGAAGCCTA | lung squamous cell carcinoma |
| 518 | GCGTACCAGGGGAATCC | lung squamous cell carcinoma |
| 519 | GCTCGAGCGTCGGGCC | lung squamous cell carcinoma |
| 520 | GCTGGCGCGTCGGGCCCAT | lung squamous cell carcinoma |
| 521 | GGAAGGCAGCAGGCGCGCA | lung squamous cell carcinoma |
| 522 | GGAGCCCGAGGCCTCTCCAG | lung squamous cell carcinoma |
| 523 | GGATCGGTCGGGCGGCGGCGG | lung squamous cell carcinoma |
| 524 | GGATCGTCGGGCCCATAC | lung squamous cell carcinoma |
| 525 | GGATTGTGGGTTCGTG | lung squamous cell carcinoma |
| 526 | GGCACGGTGAAGAGACATGA | lung squamous cell carcinoma |
| 527 | GGCCGACTCTTAGCGGTGG | lung squamous cell carcinoma |
| 528 | GGCGCGCGCCTTGGGGACCGGG | lung squamous cell carcinoma |
| 529 | GGCTGGGTTTCGTACGTAG | lung squamous cell carcinoma |
| 530 | GGGGGATTATGACTGAACGCC | lung squamous cell carcinoma |
| 531 | GTATTTGGATCTTGG | lung squamous cell carcinoma |
| 532 | GTCCCGGACACGGACAGGATTG | lung squamous cell carcinoma |
| 533 | GTCCGGGGGGCGGGGCGGACTGT | lung squamous cell carcinoma |
| 534 | GTCGAGCCGGGCGTGGAAT | lung squamous cell carcinoma |
| 535 | GTCGCGGACCAGGGGAATCCGA | lung squamous cell carcinoma |
| 536 | GTCGGGGTTTCGTAC | lung squamous cell carcinoma |
| 537 | GTCTCGATTCCCGGTCAGGGAACCA | lung squamous cell carcinoma |
| 538 | GTCTCGGGTTCATATCCCGGA | lung squamous cell carcinoma |
| 539 | GTCTGGGCGGCGGCGGCGGCGGC | lung squamous cell carcinoma |
| 540 | GTCTGGGTGGTTCAGTGGTAGAATTCTCG | lung squamous cell carcinoma |
| 541 | GTGAATCTGACAACAGAGG | lung squamous cell carcinoma |
| 542 | GTGATAACTGGCTTGTGG | lung squamous cell carcinoma |
| 543 | GTGATCGGGGATTGCA | lung squamous cell carcinoma |
| 544 | GTGGGCGTCGGAGGG | lung squamous cell carcinoma |
| 545 | GTGGTTAGTAATCTGCGTT | lung squamous cell carcinoma |
| 546 | GTGTCAGTCGGTCCTGAGA | lung squamous cell carcinoma |
| 547 | TACTTTATCACACTGATGTTGAC | lung squamous cell carcinoma |
| 548 | TAGCTGGATCTTGGG | lung squamous cell carcinoma |
| 549 | TAGCTTATCAGACTGATGTTCTC | lung squamous cell carcinoma |
| 550 | TCAACTGCTCAGACTCCTGTGG | lung squamous cell carcinoma |
| 551 | TCAGCCGGCCCGGACACG | lung squamous cell carcinoma |
| 552 | TCAGTCATTCCACCGGAG | lung squamous cell carcinoma, thymoma |
| 553 | TCAGTGTTTATCTAGCTGTATGA | lung squamous cell carcinoma |
| 554 | TCCCGCCGTCGGGCCCGGGGGAG | lung squamous cell carcinoma |
| 555 | TCCGACGACTCTTAGCGGT | lung squamous cell carcinoma |
| 556 | TCCGACGACTCTTAGCGGTGGAT | lung squamous cell carcinoma |
| 557 | TCCGATGGTAGTGGGTTATCAGAA | lung squamous cell carcinoma |
| 558 | TCCGCCCAGTGCTCTGAATG | lung squamous cell carcinoma |
| 559 | TCCGGCGAGGGAGCCTGA | lung squamous cell carcinoma |
| 560 | TCCGGGCACTACAGACCTTTGT | lung squamous cell carcinoma |
| 561 | TCTGTGTTTATCTAGCTGTAT | lung squamous cell carcinoma |
| 562 | TCTGTGTTTATCTAGCTGTAT | lung squamous cell carcinoma |
| 563 | TGAAAGAAAACTATTCTGAGC | lung squamous cell carcinoma |
| 564 | TGAAGGACACATTGATCATCG | lung squamous cell carcinoma |
| 565 | TGACGGAGAGGGAGCCTGAGA | lung squamous cell carcinoma |
| 566 | TGAGCCATGGTTGTCTGAGCATGC | lung squamous cell carcinoma |
| 567 | TGATGTAATGATTCTGCCAAATGAAATAT | lung squamous cell carcinoma |
| 568 | TGCCTCTAGTCTGGCACGGTG | lung squamous cell carcinoma |
| 569 | TGCGTGGGGAACCTGGCGCT | lung squamous cell carcinoma |
| 570 | TGCTAGTGTGCTGATG | lung squamous cell carcinoma |
| 571 | TGGAACAATGTAGGTAAGGGAA | lung squamous cell carcinoma |
| 572 | TGGACTGAGCCATGCTACTGG | lung squamous cell carcinoma |
| 573 | TGGTGCGTCGGGCCCATACCC | lung squamous cell carcinoma |
| 574 | TGTGGCAATGGTAGACCTCACACTG | lung squamous cell carcinoma |
| 575 | TGTGGCAATGGTAGACCTCACACTGG | lung squamous cell carcinoma |
| 576 | TTACCAGACTGATGTTGAC | lung squamous cell carcinoma |
| 577 | TTACGGAGAGGGAGCCTGAG | lung squamous cell carcinoma |
| 578 | TTATCATACTGATGTTGA | lung squamous cell carcinoma |
| 579 | TTCAATGTTTAGGACCACTAA | lung squamous cell carcinoma |
| 580 | TTCCAGTGTCTTAGCTGGTTG | lung squamous cell carcinoma |
| 581 | TTCCCAGACTGATGTTGA | lung squamous cell carcinoma |
| 582 | TTCGTCTTTGCGGGCGAGATG | lung squamous cell carcinoma |
| 583 | TTCGTGCATTTATCAGATCAAAA | lung squamous cell carcinoma |
| 584 | TTCGTTTACGTTGGGAGAACT | lung squamous cell carcinoma |
| 585 | TTCGTTTACGTTGGGAGAACTT | lung squamous cell carcinoma |
| 586 | TTCTGACCAGGGGAAT | lung squamous cell carcinoma |
| 587 | TTCTGACCAGGGGAATCC | lung squamous cell carcinoma |
| 588 | TTCTGGATAAGGATTGGCT | lung squamous cell carcinoma |
| 589 | TTCTTTATCAGACTGATGTGGAC | lung squamous cell carcinoma |
| 590 | TTGAGCCGGGCGTGGAA | lung squamous cell carcinoma |
| 591 | TTGCCTCTAAGGGCTGGG | lung squamous cell carcinoma |
| 592 | TTGGATTATGACTGAACGCCTC | lung squamous cell carcinoma |
| 593 | TTTTGTGTGCGTCGGAGGGC | lung squamous cell carcinoma |
| 594 | AAAAGCTGGGTTGCGAGGGAAT | ovarian serous cystadenocarcinoma |
| 595 | AAAATGGCGGATTCCTCGG | ovarian serous cystadenocarcinoma |
| 596 | AAAGAGCGCCCGGACCTG | ovarian serous cystadenocarcinoma |
| 597 | AACGGGGCGCGGCCGGAAA | ovarian serous cystadenocarcinoma |
| 598 | AACTCCAGAGAGGCCGGGCAG | ovarian serous cystadenocarcinoma |
| 599 | AACTGAGGCTCTGAGGA | ovarian serous cystadenocarcinoma |
| 600 | AAGAAAAGGACCAGGGAAGG | ovarian serous cystadenocarcinoma |
| 601 | AAGAAAGTCGACAGAGACA | ovarian serous cystadenocarcinoma |
| 602 | AAGATCGCGGGCAGATG | ovarian serous cystadenocarcinoma |
| 603 | AAGATGATCAGGTTGATAGGT | ovarian serous cystadenocarcinoma |
| 604 | AAGCAGATCTAAGGATGAGTATGAGAA | ovarian serous cystadenocarcinoma |
| 605 | AAGCTCAGCAGGGTCGGGCCTG | ovarian serous cystadenocarcinoma |
| 606 | AAGGCAGGGCCCCCGCTCCAT | ovarian serous cystadenocarcinoma |
| 607 | AAGGTCAGGGCCCAGGG | ovarian serous cystadenocarcinoma |
| 608 | AAGTGATTCTCCTGCCTCAGC | ovarian serous cystadenocarcinoma |
| 609 | AAGTGCACTTGGACGAAAA | ovarian serous cystadenocarcinoma |
| 610 | AATACCGGGTGCTGTAGGCTCTG | ovarian serous cystadenocarcinoma |
| 611 | AATACCGGGTGGTGTAGGC | ovarian serous cystadenocarcinoma |
| 612 | AATACCGGGTGGTGTAGGCTA | ovarian serous cystadenocarcinoma |
| 613 | AATCGGTCGTCCGCCCTGG | ovarian serous cystadenocarcinoma |
| 614 | AATGGCTGGCGCTGGAG | ovarian serous cystadenocarcinoma |
| 615 | ACAGAAACCTCCCGTG | ovarian serous cystadenocarcinoma |
| 616 | ACAGATCGACTATGTTGAT | ovarian serous cystadenocarcinoma |
| 617 | ACAGTCCGCCGATCTG | ovarian serous cystadenocarcinoma |
| 618 | ACCCCCGGGCGCCTG | ovarian serous cystadenocarcinoma |
| 619 | ACCCTGCTCGCTGCGCCTAT | ovarian serous cystadenocarcinoma |
| 620 | ACCCTGGTCGCACTGCTG | ovarian serous cystadenocarcinoma |
| 621 | ACCGACGTCGGCGCGGGCTGCA | ovarian serous cystadenocarcinoma |
| 622 | ACCTCAGATCGGACGTGGCGAC | ovarian serous cystadenocarcinoma |
| 623 | ACCTCGCCGTCCCGCCCGCCGC | ovarian serous cystadenocarcinoma |
| 624 | ACGATGAAGGTCTTCGGATT | ovarian serous cystadenocarcinoma |
| 625 | ACGCCAGGGTAGTGGATAGG | ovarian serous cystadenocarcinoma |
| 626 | ACGCCTCCTCCCCTCCTG | ovarian serous cystadenocarcinoma |
| 627 | ACGCTCGTGAAGGAACTCTG | ovarian serous cystadenocarcinoma |
| 628 | ACGGCTGTGGGGCAGAGAAG | ovarian serous cystadenocarcinoma |
| 629 | ACGTCGGCTGCTGGGAA | ovarian serous cystadenocarcinoma |
| 630 | ACTACCAGGGCCGGAGGATT | ovarian serous cystadenocarcinoma |
| 631 | ACTACTGCACTTGCCTAG | ovarian serous cystadenocarcinoma |
| 632 | ACTAGAACTCAGAATTAAGA | ovarian serous cystadenocarcinoma |
| 633 | ACTCAGAGGCCAGCGGACCGG | ovarian serous cystadenocarcinoma |
| 634 | ACTCAGCTTCGGACACCCTGG | ovarian serous cystadenocarcinoma |
| 635 | ACTCCCTGGGGTTGGCCGG | ovarian serous cystadenocarcinoma |
| 636 | ACTCGATCCTAAGACATAG | ovarian serous cystadenocarcinoma |
| 637 | ACTGCCGGGTACTGATGG | ovarian serous cystadenocarcinoma |
| 638 | ACTGTCTGGTCTTCTGCTTGT | ovarian serous cystadenocarcinoma |
| 639 | ACTGTGATGAGCTTCTGCAGT | ovarian serous cystadenocarcinoma |
| 640 | AGAAGATGGGCAGGGCTGTGG | ovarian serous cystadenocarcinoma |
| 641 | AGAGAACGCGGTCTGGTGGT | ovarian serous cystadenocarcinoma |
| 642 | AGCAGTGGAAGAAGACTGAAT | ovarian serous cystadenocarcinoma |
| 643 | AGCGCCGGAGAGGACCACTGA | ovarian serous cystadenocarcinoma |
| 644 | AGCGGTGGTCTGTGGAGCTC | ovarian serous cystadenocarcinoma |
| 645 | AGCTCCGGGGAGAGCATTTGACT | ovarian serous cystadenocarcinoma |
| 646 | AGCTGGCCGGGTTGGAGG | ovarian serous cystadenocarcinoma |
| 647 | AGCTGTGGTCTCTGAGAGA | ovarian serous cystadenocarcinoma |
| 648 | AGCTTGACTGTGAGA | ovarian serous cystadenocarcinoma |
| 649 | AGGACTGTGAGCCGGCTGC | ovarian serous cystadenocarcinoma |
| 650 | AGGAGCTCTGGGCCCACCCAT | ovarian serous cystadenocarcinoma, thymoma |
| 651 | AGGAGCTGGAGGATCTGG | ovarian serous cystadenocarcinoma |
| 652 | AGGAGGTCGTCGTGGCGC | ovarian serous cystadenocarcinoma |
| 653 | AGGCCAAGAGCAGGAGAGA | ovarian serous cystadenocarcinoma |
| 654 | AGGCCGAGAGCGAAGCGCG | ovarian serous cystadenocarcinoma |
| 655 | AGGCCTGCAGCGAACTCTGG | ovarian serous cystadenocarcinoma |
| 656 | AGGCGGACGGACTGAGGCTG | ovarian serous cystadenocarcinoma |
| 657 | AGGCTGTAGGCACCACGCA | ovarian serous cystadenocarcinoma |
| 658 | AGGGAACCTGGGCTGCAG | ovarian serous cystadenocarcinoma |
| 659 | AGGGACTGACGGGGGCATT | ovarian serous cystadenocarcinoma |
| 660 | AGGGACTGTGGCCTCGGCGGTAT | ovarian serous cystadenocarcinoma |
| 661 | AGGGAGAGAGTCGGACGA | ovarian serous cystadenocarcinoma |
| 662 | AGGGATTGTGGAGCTCTGG | ovarian serous cystadenocarcinoma |
| 663 | AGGGCCCCCCCTCAATCCCGT | ovarian serous cystadenocarcinoma |
| 664 | AGGGCCGTCGCGGTG | ovarian serous cystadenocarcinoma |
| 665 | AGGGCTGCGCATTGCACTGGCC | ovarian serous cystadenocarcinoma |
| 666 | AGGGCTGCGGCAGGGACT | ovarian serous cystadenocarcinoma |
| 667 | AGGGTGAAGGATGTCGGTTGG | ovarian serous cystadenocarcinoma |
| 668 | AGGGTTCGTGACTGGGGT | ovarian serous cystadenocarcinoma |
| 669 | AGGTCGCGGAGAGAAGGGG | ovarian serous cystadenocarcinoma |
| 670 | AGGTCGGCAGGGCTGGTG | ovarian serous cystadenocarcinoma |
| 671 | AGGTGATCGAGAGAACTCT | ovarian serous cystadenocarcinoma |
| 672 | AGGTGGACCCGGGCTGTG | ovarian serous cystadenocarcinoma |
| 673 | AGGTGGTGGGACTGGGGGC | ovarian serous cystadenocarcinoma |
| 674 | AGTCCACGATCTGGTCAC | ovarian serous cystadenocarcinoma |
| 675 | AGTCCGACGATCTGATCCA | ovarian serous cystadenocarcinoma |
| 676 | AGTCGAACGCTTGGCCAGAT | ovarian serous cystadenocarcinoma |
| 677 | AGTCGCAGGAGCAGCCGCAGTAG | ovarian serous cystadenocarcinoma |
| 678 | ATAATACTGCCGGGTAATGAT | ovarian serous cystadenocarcinoma |
| 679 | ATACCGGTTGCTGTAGGCTA | ovarian serous cystadenocarcinoma |
| 680 | ATAGGAAACCGAACTCATGC | ovarian serous cystadenocarcinoma |
| 681 | ATATCGCCGGGCCGGCCGC | ovarian serous cystadenocarcinoma |
| 682 | ATCGAGGCCCAGCCCGTG | ovarian serous cystadenocarcinoma |
| 683 | ATGCCATCACGGCAGCGCTCTGACC | ovarian serous cystadenocarcinoma |
| 684 | ATGCGAACCGCGTGGCTGGG | ovarian serous cystadenocarcinoma |
| 685 | ATGCTATGACTGTATCTTGAT | ovarian serous cystadenocarcinoma |
| 686 | ATGCTCAACATCTCCCCCTT | ovarian serous cystadenocarcinoma |
| 687 | ATGGAGCCGGGCGTGG | ovarian serous cystadenocarcinoma |
| 688 | ATGGCAGCCAGGTAGTCAGG | ovarian serous cystadenocarcinoma |
| 689 | ATGGGCGAGCGCCGTTCCGA | ovarian serous cystadenocarcinoma |
| 690 | ATGTGCGGACGCCGGGAGCTT | ovarian serous cystadenocarcinoma |
| 691 | ATTAATAAATTCTGATCAG | ovarian serous cystadenocarcinoma |
| 692 | ATTCCAGGGTTCGGTTGCG | ovarian serous cystadenocarcinoma |
| 693 | ATTGGCTCTAAGGGCTGGGTCGGGCGGG | ovarian serous cystadenocarcinoma |
| 694 | ATTGGTGGCTCTCGCCGGGCAGC | ovarian serous cystadenocarcinoma |
| 695 | CAAACGAGCACTTTGA | ovarian serous cystadenocarcinoma |
| 696 | CAACATCTCTGCCAAGT | ovarian serous cystadenocarcinoma |
| 697 | CAACCCAGATCACCCGCTAAG | ovarian serous cystadenocarcinoma |
| 698 | CAAGAACCGAGAGCTCTTC | ovarian serous cystadenocarcinoma |
| 699 | CAAGCCGGATCCGTACCTTCGGGA | ovarian serous cystadenocarcinoma |
| 700 | CAAGGAAGAAGTGGGCTGAAG | ovarian serous cystadenocarcinoma |
| 701 | CAAGGCGACGATCTGTAG | ovarian serous cystadenocarcinoma |
| 702 | CAAGTGTTGGATTGTT | ovarian serous cystadenocarcinoma |
| 703 | CAATACCCCGCCATGACGAC | ovarian serous cystadenocarcinoma |
| 704 | CACACCGGATGTCATCTTTGT | ovarian serous cystadenocarcinoma |
| 705 | CACAGAGGCCGGGGAGAG | ovarian serous cystadenocarcinoma |
| 706 | CACCAAGATCTCTGATGCAC | ovarian serous cystadenocarcinoma |
| 707 | CACCGTGATGACCAGGTCGGTCTCAAATTT | ovarian serous cystadenocarcinoma |
| 708 | CACCTCGCGGACTCCATG | ovarian serous cystadenocarcinoma |
| 709 | CACCTGACCTCCCCTGCCTGTGT | ovarian serous cystadenocarcinoma |
| 710 | CACGATCCTTCTGACCTT | ovarian serous cystadenocarcinoma |
| 711 | CACGCGTCGGGCCCCCA | ovarian serous cystadenocarcinoma |
| 712 | CACTGCCCCAGGTGCTGCT | ovarian serous cystadenocarcinoma |
| 713 | CACTGGGACTGAGCCACGGC | ovarian serous cystadenocarcinoma |
| 714 | CAGAAGGAAGGATTGTGGGG | ovarian serous cystadenocarcinoma |
| 715 | CAGCAGCACTCGCCGAA | ovarian serous cystadenocarcinoma |
| 716 | CAGCTCCAGAACCGACTCCG | ovarian serous cystadenocarcinoma |
| 717 | CAGCTCCCTCGCTGCGATCTAT | ovarian serous cystadenocarcinoma |
| 718 | CAGCTGTAGATTTCAACTTAG | ovarian serous cystadenocarcinoma |
| 719 | CAGGAGCGAGCAGCCAAGGG | ovarian serous cystadenocarcinoma |
| 720 | CAGGCGGCTCGGACTGA | ovarian serous cystadenocarcinoma |
| 721 | CAGTCCGACCATCTGGTCAC | ovarian serous cystadenocarcinoma |
| 722 | CATACAGACTAGGCCGGGCG | ovarian serous cystadenocarcinoma |
| 723 | CATCCCGGACGAGCCCTG | ovarian serous cystadenocarcinoma |
| 724 | CATCCGACGATCTGGTCA | ovarian serous cystadenocarcinoma |
| 725 | CATGCAAGTCGAGCGGACTTT | ovarian serous cystadenocarcinoma |
| 726 | CATGCTGACCTCCCTCCTGCCCCAAA | ovarian serous cystadenocarcinoma |
| 727 | CATGGGTCGGAAAAAAGGATT | ovarian serous cystadenocarcinoma |
| 728 | CATGTGGATGACTCTGTG | ovarian serous cystadenocarcinoma |
| 729 | CATTGCACTCCGGATGTGC | ovarian serous cystadenocarcinoma |
| 730 | CCAAGATCGGGGACCCGGC | ovarian serous cystadenocarcinoma |
| 731 | CCACGAGGTCTGAGTGGT | ovarian serous cystadenocarcinoma |
| 732 | CCACTAATAAACATAGTAGTCTTA | ovarian serous cystadenocarcinoma |
| 733 | CCAGGCGGCCCGGGTTCGTCTCCCGGTGTG | ovarian serous cystadenocarcinoma |
| 734 | CCATTGATGATCGTTCT | ovarian serous cystadenocarcinoma |
| 735 | CCCACCGCGGACAGAGCCTGA | ovarian serous cystadenocarcinoma |
| 736 | CCCACCTCGGCCTCC | ovarian serous cystadenocarcinoma |
| 737 | CCCAGAACTCCGGCCGTGTG | ovarian serous cystadenocarcinoma |
| 738 | CCCAGAAGTCCAAGTTATGGAAGATCTCG | ovarian serous cystadenocarcinoma |
| 739 | CCCCCGCCGCCCCTCCTA | ovarian serous cystadenocarcinoma |
| 740 | CCCCCGCCGCCCCTCCTCCTA | ovarian serous cystadenocarcinoma |
| 741 | CCCGCCGGGGCCCGTCGTA | ovarian serous cystadenocarcinoma |
| 742 | CCCGCGTCGGCTGCGCAGCGC | ovarian serous cystadenocarcinoma |
| 743 | CCCGCTGGTGAGGACTCGCG | ovarian serous cystadenocarcinoma |
| 744 | CCCGGGTCGAGCATGTAGCG | ovarian serous cystadenocarcinoma |
| 745 | CCCGTCCGTCCGTCCGTA | ovarian serous cystadenocarcinoma |
| 746 | CCCTGAGGATGACAGAATAAGAACTGAG | ovarian serous cystadenocarcinoma |
| 747 | CCCTGGGCCGGGGAGGAGTGGA | ovarian serous cystadenocarcinoma |
| 748 | CCCTTCCTGGCCGCCGGGCTG | ovarian serous cystadenocarcinoma |
| 749 | CCGAATCCGGAGTGGCG | ovarian serous cystadenocarcinoma |
| 750 | CCGCCCCCCGGCCCCC | ovarian serous cystadenocarcinoma |
| 751 | CCGGCAGATTGTCTCTGG | ovarian serous cystadenocarcinoma |
| 752 | CCGTTCCCCCCTCCTAT | ovarian serous cystadenocarcinoma |
| 753 | CCGTTCCCCCCTCCTTA | ovarian serous cystadenocarcinoma |
| 754 | CCGTTTCGCCGGACTCTTCGC | ovarian serous cystadenocarcinoma |
| 755 | CCTACCTTGAGCACATCTTTGAG | ovarian serous cystadenocarcinoma |
| 756 | CCTAGTCAAGGGTTAGATTTTTATT | ovarian serous cystadenocarcinoma |
| 757 | CCTCACTGAGATCAGGGACATGTTGCTGGC | ovarian serous cystadenocarcinoma |
| 758 | CCTGCTGGTCCCACTGGAGCTCG | ovarian serous cystadenocarcinoma |
| 759 | CCTGGCGGCCGGGGGCGAT | ovarian serous cystadenocarcinoma |
| 760 | CCTTCGTCCTGGGAA | ovarian serous cystadenocarcinoma |
| 761 | CGAATACAGACCGTGAAAG | ovarian serous cystadenocarcinoma |
| 762 | CGAGAGGGCGCGAGCGGCGGCG | ovarian serous cystadenocarcinoma |
| 763 | CGAGGCCGCGGCGGAGGC | ovarian serous cystadenocarcinoma |
| 764 | CGAGTTGTTGCCTGGGCTGG | ovarian serous cystadenocarcinoma |
| 765 | CGATCGCCGCCGGGACCTGA | ovarian serous cystadenocarcinoma |
| 766 | CGCACAGTTAACTGGAACTCT | ovarian serous cystadenocarcinoma |
| 767 | CGCCGCGGGGCCGGCGGCGGGGGC | ovarian serous cystadenocarcinoma |
| 768 | CGCCTAGCAGCCGACTTCG | ovarian serous cystadenocarcinoma |
| 769 | CGCGCCAGGCAGTCGTCGGTAA | ovarian serous cystadenocarcinoma |
| 770 | CGCGCTGAGGCTCCAGGACC | ovarian serous cystadenocarcinoma |
| 771 | CGCGGACTGGGGCGGCCA | ovarian serous cystadenocarcinoma |
| 772 | CGCGGCGGCCGTCGGGTGGGGGCGT | ovarian serous cystadenocarcinoma |
| 773 | CGCGGGCGGGCGCGACGGA | ovarian serous cystadenocarcinoma |
| 774 | CGCGGGGATGCGAACTGGAGTCTGAGC | ovarian serous cystadenocarcinoma |
| 775 | CGGAGGGCGGCGGCGGCGGCGGGGGGGTGG | ovarian serous cystadenocarcinoma |
| 776 | CGGAGGTTCGAAGACGATCCG | ovarian serous cystadenocarcinoma |
| 777 | CGGATCGCCGGTCCCCCGCCTGT | ovarian serous cystadenocarcinoma |
| 778 | CGGATCGCGTGGGTAGAAGGTC | ovarian serous cystadenocarcinoma |
| 779 | CGGCCGCGTCGGGGC | ovarian serous cystadenocarcinoma |
| 780 | CGGCGCACCCCCGCGGGGA | ovarian serous cystadenocarcinoma |
| 781 | CGGCGCACTGAGGACTGTGGT | ovarian serous cystadenocarcinoma |
| 782 | CGGCGCCGGGCGAGGCTGTGCG | ovarian serous cystadenocarcinoma |
| 783 | CGGCGCGCGCCGGCGGCGGCGGC | ovarian serous cystadenocarcinoma |
| 784 | CGGCGCGCGGCGGCGGCGGT | ovarian serous cystadenocarcinoma |
| 785 | CGGCGCTGGACTGGAGACC | ovarian serous cystadenocarcinoma |
| 786 | CGGCTGGGGGCCCTGGGTACG | ovarian serous cystadenocarcinoma |
| 787 | CGGCTGTGGTGCTGGCGGCGAC | ovarian serous cystadenocarcinoma |
| 788 | CGGGATCTGGCTGCGCTATGACT | ovarian serous cystadenocarcinoma |
| 789 | CGGGCCGGGCGACTTGAGA | ovarian serous cystadenocarcinoma |
| 790 | CGGGCGGACGAGCGGGCGGG | ovarian serous cystadenocarcinoma |
| 791 | CGGGGACTGTGAGGCTGC | ovarian serous cystadenocarcinoma |
| 792 | CGGGTGACTGGGCGCCGG | ovarian serous cystadenocarcinoma |
| 793 | CGGTAGAGCATCTGACTTT | ovarian serous cystadenocarcinoma |
| 794 | CGGTCTAAGGGGCTGCGTTC | ovarian serous cystadenocarcinoma |
| 795 | CGGTGCCAGACTAGGACG | ovarian serous cystadenocarcinoma |
| 796 | CGTACCAGAGCTTGCGGCTGGAGT | ovarian serous cystadenocarcinoma |
| 797 | CGTGATCCGCCCGCCTCGGC | ovarian serous cystadenocarcinoma |
| 798 | CGTGCCTGAGGTTTCTCCCCGA | ovarian serous cystadenocarcinoma |
| 799 | CGTGGCGGACTCTGGGAC | ovarian serous cystadenocarcinoma |
| 800 | CGTGGGGCTGCTTAGATG | ovarian serous cystadenocarcinoma, thyroid |
| | | carcinoma |
| 801 | CGTTCCTCGGCTTGTAG | ovarian serous cystadenocarcinoma |
| 802 | CTAAGGCTGGGTCGGTCGGGC | ovarian serous cystadenocarcinoma |
| 803 | CTAGACTGCAGCTCCTTGAGGTG | ovarian serous cystadenocarcinoma |
| 804 | CTAGAGCTCTCCGGCGGCGGCG | ovarian serous cystadenocarcinoma |
| 805 | CTAGCGCCGATGGTAGTTGG | ovarian serous cystadenocarcinoma |
| 806 | CTAGGACGTGTCGGGCTGC | ovarian serous cystadenocarcinoma |
| 807 | CTATCATTGTGAAGCAGAATTC | ovarian serous cystadenocarcinoma |
| 808 | CTCAGAACGAACGTTGGC | ovarian serous cystadenocarcinoma |
| 809 | CTCAGCTTTGGGTGGTGGCCG | ovarian serous cystadenocarcinoma |
| 810 | CTCCAGGCCAGGTGGGAC | ovarian serous cystadenocarcinoma |
| 811 | CTCCCACGGCGCGCCCGCT | ovarian serous cystadenocarcinoma |
| 812 | CTCCCTCGCTGCGATCTATTGAA | ovarian serous cystadenocarcinoma |
| 813 | CTCGCCCTGGAGTCTCGG | ovarian serous cystadenocarcinoma |
| 814 | CTCGGCGGAGCTGTAAGATGGC | ovarian serous cystadenocarcinoma |
| 815 | CTCGGTGGGGGCCGAGGGCCG | ovarian serous cystadenocarcinoma |
| 816 | CTCGTCGCGCGCGCGTCCGCAT | ovarian serous cystadenocarcinoma |
| 817 | CTCGTCGGCCTGCGGCGGC | ovarian serous cystadenocarcinoma |
| 818 | CTCGTCTGATCTCGGCAG | ovarian serous cystadenocarcinoma |
| 819 | CTCTCTTGGACTGCCACGGC | ovarian serous cystadenocarcinoma |
| 820 | CTCTGAAATACAAAATGAAA | ovarian serous cystadenocarcinoma |
| 821 | CTCTGAATCCAGTCTGTCCTT | ovarian serous cystadenocarcinoma |
| 822 | CTCTGAATGTCAACGTGAAGA | ovarian serous cystadenocarcinoma |
| 823 | CTCTGAGGCAAGCCAGGACC | ovarian serous cystadenocarcinoma |
| 824 | CTCTGCGTGCGCCGGCGGCTGC | ovarian serous cystadenocarcinoma |
| 825 | CTCTGGACCAGACGCCGCCTG | ovarian serous cystadenocarcinoma |
| 826 | CTCTGTCTCTGTCTCT | ovarian serous cystadenocarcinoma |
| 827 | CTCTGTGTCTCCCACCGGC | ovarian serous cystadenocarcinoma |
| 828 | CTGACCTTCGTGCCCCGCCAGA | ovarian serous cystadenocarcinoma |
| 829 | CTGATATCTCATTCCTGAACACTC | ovarian serous cystadenocarcinoma |
| 830 | CTGATCTCTGAAGCTAAGCAG | ovarian serous cystadenocarcinoma |
| 831 | CTGCAGGTGGGTCGGATGAGGCT | ovarian serous cystadenocarcinoma |
| 832 | CTGCCGATCGCGGGCCCGGC | ovarian serous cystadenocarcinoma |
| 833 | CTGCGCCCCCAGTTGGCTCTG | ovarian serous cystadenocarcinoma |
| 834 | CTGCGGACCGAGCGGCGAGC | ovarian serous cystadenocarcinoma |
| 835 | CTGCGGCTGTGGGTTCTCTGC | ovarian serous cystadenocarcinoma |
| 836 | CTGCTGATCGGGGACTCG | ovarian serous cystadenocarcinoma |
| 837 | CTGCTGGCCCTCTCCTGGTGC | ovarian serous cystadenocarcinoma |
| 838 | CTGCTGGTCCCACTGGAGC | ovarian serous cystadenocarcinoma |
| 839 | CTGGAGCGTCGGGCCATACC | ovarian serous cystadenocarcinoma |
| 840 | CTGGATCGGCGGCGCGGCG | ovarian serous cystadenocarcinoma |
| 841 | CTGGATGATGATACGCAAATGCTG | ovarian serous cystadenocarcinoma |
| 842 | CTGGCATGTTGGACCAATGTAGG | ovarian serous cystadenocarcinoma |
| 843 | CTGGCCCCTGACCCCTCCCTGGC | ovarian serous cystadenocarcinoma |
| 844 | CTGGCTCAGGACTCTGCAGC | ovarian serous cystadenocarcinoma |
| 845 | CTGGCTCTCTGGATGGTAGGC | ovarian serous cystadenocarcinoma |
| 846 | CTGGCTGGGGCTCGTGTGGCAGT | ovarian serous cystadenocarcinoma |
| 847 | CTGGGACACGGGGAACTG | ovarian serous cystadenocarcinoma |
| 848 | CTGGGCTGGGAGCAACGCGAC | ovarian serous cystadenocarcinoma |
| 849 | CTGGGGTTGTAGGGCGT | ovarian serous cystadenocarcinoma |
| 850 | CTGGTAGGTGACTGGAGG | ovarian serous cystadenocarcinoma |
| 851 | CTGTAGCTGGGCAGGGAGAGGG | ovarian serous cystadenocarcinoma |
| 852 | CTGTCCTATGAGCTCTAGCA | ovarian serous cystadenocarcinoma |
| 853 | CTGTCGGTGTAACTTCTTAGA | ovarian serous cystadenocarcinoma |
| 854 | CTGTCTGAATCTTTAACCGAC | ovarian serous cystadenocarcinoma |
| 855 | CTGTGCACAGAGTTGTCTGCCC | ovarian serous cystadenocarcinoma |
| 856 | CTTAACCACCATTATCTTAACTG | ovarian serous cystadenocarcinoma |
| 857 | CTTATTTTGGTAAGTTGATCTTCAAT | ovarian serous cystadenocarcinoma |
| 858 | CTTCGATGAAGAGATGATGCCGAGTCTGAC | ovarian serous cystadenocarcinoma |
| 859 | CTTCGGGTTGTAAAG | ovarian serous cystadenocarcinoma |
| 860 | CTTGTAATTGCCATTCATT | ovarian serous cystadenocarcinoma |
| 861 | GAAAATGAAGAAGGAACG | ovarian serous cystadenocarcinoma |
| 862 | GAAACATCGGATTTGGGGAACGCGT | ovarian serous cystadenocarcinoma |
| 863 | GAACAGTCGGAACGCGGTGG | ovarian serous cystadenocarcinoma |
| 864 | GAACTTAGGTCGCCGGGACG | ovarian serous cystadenocarcinoma |
| 865 | GAAGTGATCTGGGGCTGGAGC | ovarian serous cystadenocarcinoma |
| 866 | GAATCCTGCTCACAGCGCCA | ovarian serous cystadenocarcinoma |
| 867 | GAATGTTTTCATTAATCAAGAACGAAAGTC | ovarian serous cystadenocarcinoma |
| 868 | GACACTCTGCGACCGTG | ovarian serous cystadenocarcinoma |
| 869 | GACCCCCATCTGTGCCTCCAGT | ovarian serous cystadenocarcinoma |
| 870 | GACCTGCTTCTTTGACCCAG | ovarian serous cystadenocarcinoma |
| 871 | GAGAAGCTCTGAATCGAAG | ovarian serous cystadenocarcinoma |
| 872 | GAGACTGACCCGGACTTAGG | ovarian serous cystadenocarcinoma |
| 873 | GAGACTGGCCCTGCTGGAC | ovarian serous cystadenocarcinoma |
| 874 | GAGAGCTGTTGTACTGGGTGG | ovarian serous cystadenocarcinoma |
| 875 | GAGCCGGGCGTGGCA | ovarian serous cystadenocarcinoma |
| 876 | GAGCGGTGGTCTGTGGAGCTCGG | ovarian serous cystadenocarcinoma |
| 877 | GAGCTCGTTGTGGAACCTG | ovarian serous cystadenocarcinoma |
| 878 | GAGCTGCCAAATGAAGGGCTGT | ovarian serous cystadenocarcinoma |
| 879 | GAGGAGGAGCTCTGCGCGGCG | ovarian serous cystadenocarcinoma |
| 880 | GAGGCAGGGGGGCGGGCGA | ovarian serous cystadenocarcinoma |
| 881 | GAGGCTGTAGGGACCGACGG | ovarian serous cystadenocarcinoma |
| 882 | GAGGTAGAGCTACTGATTGG | ovarian serous cystadenocarcinoma |
| 883 | GAGGTCGGACCCGGGGAG | ovarian serous cystadenocarcinoma |
| 884 | GAGGTCGGGGTGGGGAGA | ovarian serous cystadenocarcinoma |
| 885 | GAGTATCTGGGGCTGTTCTGG | ovarian serous cystadenocarcinoma |
| 886 | GAGTCGCCGGAGGAGCCAG | ovarian serous cystadenocarcinoma |
| 887 | GAGTCGGGTTGCTTGGGC | ovarian serous cystadenocarcinoma |
| 888 | GAGTGACTGGGCGGTAGA | ovarian serous cystadenocarcinoma |
| 889 | GAGTGATTTGTCTGG | ovarian serous cystadenocarcinoma |
| 890 | GATAGGCCGGGTGTGGAAGCG | ovarian serous cystadenocarcinoma |
| 891 | GATCATTAACGGAGCCCGGAGG | ovarian serous cystadenocarcinoma |
| 892 | GATTTCTGCCCCGTGCTCTG | ovarian serous cystadenocarcinoma |
| 893 | GCAGACCCGGGGAACTGAACC | ovarian serous cystadenocarcinoma |
| 894 | GCAGCCAGGCTCGGAGCG | ovarian serous cystadenocarcinoma |
| 895 | GCAGCGGCTCGGGGACTGGG | ovarian serous cystadenocarcinoma |
| 896 | GCAGGTCGGAGCTCGGAGCT | ovarian serous cystadenocarcinoma |
| 897 | GCAGTCTTCAGCCTGAGA | ovarian serous cystadenocarcinoma |
| 898 | GCCCCCCCGGTGTCCCCGCA | ovarian serous cystadenocarcinoma |
| 899 | GCCGGCGACTGTGGAGTTAG | ovarian serous cystadenocarcinoma |
| 900 | GCCGTCCGGGGTCTGAAGA | ovarian serous cystadenocarcinoma |
| 901 | GCCGTGCCTGCTGTAGAGA | ovarian serous cystadenocarcinoma |
| 902 | GCCTCCGAGGACTGTGAAG | ovarian serous cystadenocarcinoma |
| 903 | GCCTCGGCCGGCGCCTAGCAGC | ovarian serous cystadenocarcinoma |
| 904 | GCCTGCGGGGTCGTCTGG | ovarian serous cystadenocarcinoma |
| 905 | GCCTTCTCTGAACACCCCGGC | ovarian serous cystadenocarcinoma |
| 906 | GCGAGCCGCTGGGAACTGAAC | ovarian serous cystadenocarcinoma |
| 907 | GCGAGCGCGGACTGGGGCG | ovarian serous cystadenocarcinoma |
| 908 | GCGCGCCGGCCGGGCGCGACCCGC | ovarian serous cystadenocarcinoma |
| 909 | GCGCGCGTGTCGCCCCCGG | ovarian serous cystadenocarcinoma |
| 910 | GCGGACTATCGGGCGGCTAG | ovarian serous cystadenocarcinoma |
| 911 | GCGGCCGGGGGCGAT | ovarian serous cystadenocarcinoma |
| 912 | GCGGGCCGGGCGACTTGAGA | ovarian serous cystadenocarcinoma |
| 913 | GCGGGCGGGCGCGACGGA | ovarian serous cystadenocarcinoma |
| 914 | GCGGGCGGGCGCGACGGAG | ovarian serous cystadenocarcinoma |
| 915 | GCGGGCGTCAGTACAGTAGAG | ovarian serous cystadenocarcinoma |
| 916 | GCGGGTTATGCGACCTGGAGTCTGAGC | ovarian serous cystadenocarcinoma |
| 917 | GCGTCGGGCCCGGGGGAGGT | ovarian serous cystadenocarcinoma |
| 918 | GCGTGTGCCACCCCTGCG | ovarian serous cystadenocarcinoma |
| 919 | GCTAATGATTAGTGGACTCG | ovarian serous cystadenocarcinoma |
| 920 | GCTCGGGCGTCGGATCGGA | ovarian serous cystadenocarcinoma |
| 921 | GCTGGGAGTGGATCTGAGA | ovarian serous cystadenocarcinoma |
| 922 | GCTGGGCTGGAGTCTCTG | ovarian serous cystadenocarcinoma |
| 923 | GCTGGGCTGGAGTCTCTGC | ovarian serous cystadenocarcinoma |
| 924 | GCTGGTCGGCGGCCGCTCTG | ovarian serous cystadenocarcinoma |
| 925 | GCTTGACATTGAACTTTGATC | ovarian serous cystadenocarcinoma |
| 926 | GGAGGTAGAGCACTGATTGG | ovarian serous cystadenocarcinoma |
| 927 | GGAGTCGGGTTCAGAT | ovarian serous cystadenocarcinoma |
| 928 | GGATCCTGCTCGGGCTG | ovarian serous cystadenocarcinoma |
| 929 | GGCGGCGGCGGGGGTGTGGGGTCT | ovarian serous cystadenocarcinoma |
| 930 | GGCTGCGGAGCCAATGGGGCGAAG | ovarian serous cystadenocarcinoma |
| 931 | GGCTGCTCTGGACTGTCCTAG | ovarian serous cystadenocarcinoma |
| 932 | GGGACTGGGAGCGGGCGG | ovarian serous cystadenocarcinoma |
| 933 | GGGAGAGGACGCGGTCTGAGTGGT | ovarian serous cystadenocarcinoma |
| 934 | GGGATATGGGACTGAGACCA | ovarian serous cystadenocarcinoma |
| 935 | GGGCAAAAGCTCGCTTGCTCTT | ovarian serous cystadenocarcinoma |
| 936 | GGGCAGCTTCCGGGAAACCAAAGT | ovarian serous cystadenocarcinoma |
| 937 | GGGCCGACCAGAAAAAGGACG | ovarian serous cystadenocarcinoma |
| 938 | GGGCGCACCACCGGCCCGTCTC | ovarian serous cystadenocarcinoma |
| 939 | GGGCGCGCAGGCAGAGAACG | ovarian serous cystadenocarcinoma |
| 940 | GGGCTGCGGGCGGCCGGGCGCGG | ovarian serous cystadenocarcinoma |
| 941 | GGGCTGCTAAGGGAACTGTGA | ovarian serous cystadenocarcinoma |
| 942 | GGGGCTGCCTCTGGAATG | ovarian serous cystadenocarcinoma |
| 943 | GGGGCTTCGGGCTGTCGGGC | ovarian serous cystadenocarcinoma |
| 944 | GGGGGAAGAGCATTTGACT | ovarian serous cystadenocarcinoma |
| 945 | GGGGGGAGAAGGGTCGGGGA | ovarian serous cystadenocarcinoma |
| 946 | GGGGGGAGCCAAAAGGGTCATC | ovarian serous cystadenocarcinoma |
| 947 | GGGGGGTAGAGCGACTGAATC | ovarian serous cystadenocarcinoma |
| 948 | GGGGGTATAGCTCAGGGTTCAGAGC | ovarian serous cystadenocarcinoma |
| 949 | GGGTCCGTCGGGCTGGGGCGC | ovarian serous cystadenocarcinoma |
| 950 | GGGTCGGGGCGGCGGCA | ovarian serous cystadenocarcinoma |
| 951 | GGTCAGTCGGTCCTAAGAG | ovarian serous cystadenocarcinoma |
| 952 | GGTCGCGCCGTTGGAGGGC | ovarian serous cystadenocarcinoma |
| 953 | GGTCGGCGGGCGGCGGGGGGGGGGGGTT | ovarian serous cystadenocarcinoma |
| 954 | GGTGAGCTCGTCGTCCGGATTGC | ovarian serous cystadenocarcinoma |
| 955 | GGTGCGGACCAGGGGCATCCGACTG | ovarian serous cystadenocarcinoma |
| 956 | GGTGGAGGCGTCGGTGGGA | ovarian serous cystadenocarcinoma |
| 957 | GGTGGGGATCGGGGATTGC | ovarian serous cystadenocarcinoma |
| 958 | GGTTCGAATCCTCCATG | ovarian serous cystadenocarcinoma |
| 959 | GTAGTTTGACTGGGGCGGT | ovarian serous cystadenocarcinoma |
| 960 | GTCGAGTGACTCTGCGCC | ovarian serous cystadenocarcinoma |
| 961 | GTCGCGGCCTGAGGAGACC | ovarian serous cystadenocarcinoma |
| 962 | GTCGGGAAAGAAGGGCTGGG | ovarian serous cystadenocarcinoma |
| 963 | GTCTGATCTCGGAAGCTAAGCAA | ovarian serous cystadenocarcinoma |
| 964 | GTGAGATCTGGGAACTCCGGGC | ovarian serous cystadenocarcinoma |
| 965 | GTGATTTCTGCCCCGTGCTCTGA | ovarian serous cystadenocarcinoma |
| 966 | GTGCGGGACCTGAGGCTGTGTG | ovarian serous cystadenocarcinoma |
| 967 | GTGCGGTGGGGGACCCTGA | ovarian serous cystadenocarcinoma |
| 968 | GTGCGTCAAGGACTACTG | ovarian serous cystadenocarcinoma |
| 969 | GTGGACAGACACAGGCCTGAGG | ovarian serous cystadenocarcinoma |
| 970 | GTGGACTCGGGCCGCTGC | ovarian serous cystadenocarcinoma |
| 971 | GTGGGGCCGGAAGGACTG | ovarian serous cystadenocarcinoma |
| 972 | GTGGGTAGCGCATTTGACTG | ovarian serous cystadenocarcinoma |
| 973 | GTGTATTTTTAGATGGAA | ovarian serous cystadenocarcinoma |
| 974 | GTGTCGCGCCGTCGGGCT | ovarian serous cystadenocarcinoma |
| 975 | GTGTTAGGACCCGCAAGATGGT | ovarian serous cystadenocarcinoma |
| 976 | GTTAGCGTTCGGATAGTTGA | ovarian serous cystadenocarcinoma |
| 977 | GTTCTCAGTTCGGATTGGAG | ovarian serous cystadenocarcinoma |
| 978 | GTTCTTCCAGTTCGCAGTCAAG | ovarian serous cystadenocarcinoma |
| 979 | GTTGGCTGGGGCTGGATG | ovarian serous cystadenocarcinoma |
| 980 | GTTTGATCTCGGAAGCTAAGC | ovarian serous cystadenocarcinoma |
| 981 | GTTTTCGGAACTGAGGCCATGATTAA | ovarian serous cystadenocarcinoma |
| 982 | GTTTTCGGATCGTAAAAC | ovarian serous cystadenocarcinoma |
| 983 | TAAGCTCTGTCGTCGAG | ovarian serous cystadenocarcinoma |
| 984 | TAAGCTTGCGTTGATTAAGTCCCTG | ovarian serous cystadenocarcinoma |
| 985 | TAAGGAACTGTCTGAGGGA | ovarian serous cystadenocarcinoma |
| 986 | TAAGGGTCGGGGCGGCAGGGGC | ovarian serous cystadenocarcinoma |
| 987 | TACGGAGCGGACGGACTGGGG | ovarian serous cystadenocarcinoma |
| 988 | TAGAGCGCCGGGACCTGTGAC | ovarian serous cystadenocarcinoma |
| 989 | TAGATCCATTCATGTTTGCAC | ovarian serous cystadenocarcinoma |
| 990 | TAGCAGCGGGACCAGTTCTGCAG | ovarian serous cystadenocarcinoma |
| 991 | TAGGCCCTGAATCAAGAC | ovarian serous cystadenocarcinoma |
| 992 | TAGTATGTGGTTGTAGAC | ovarian serous cystadenocarcinoma |
| 993 | TATCCATTGCACTCCG | ovarian serous cystadenocarcinoma |
| 994 | TATTGGCCAGTTTTGTCTGATGC | ovarian serous cystadenocarcinoma, uterine |
| | | corpus endometrial carcinoma |
| 995 | TCACTCCTCTCCTCCCGTCTTCAG | ovarian serous cystadenocarcinoma |
| 996 | TCATTGACCTCGTGTTGGACCGA | ovarian serous cystadenocarcinoma |
| 997 | TCCCCGGCACCTCCACTAAA | ovarian serous cystadenocarcinoma |
| 998 | TCCCCGGCCTCTCCACCAAGA | ovarian serous cystadenocarcinoma |
| 999 | TCCCTGAGCCCCTAACTT | ovarian serous cystadenocarcinoma |
| 1000 | TCCGCCGGGCATCGCTCTG | ovarian serous cystadenocarcinoma |
| 1001 | TCCGGGCTCTGGGTGGCGGC | ovarian serous cystadenocarcinoma |
| 1002 | TCGAGCCCAGGTCGAGG | ovarian serous cystadenocarcinoma |
| 1003 | TCGCACCCGGACCGAGACCAG | ovarian serous cystadenocarcinoma |
| 1004 | TCGGAAGCTAAGCAGGGTCGGG | ovarian serous cystadenocarcinoma |
| 1005 | TCGGTCGGGCTGGGGCGAAGC | ovarian serous cystadenocarcinoma |
| 1006 | TCGTCTGAACCTGTCTGCAGC | ovarian serous cystadenocarcinoma |
| 1007 | TCTAACTGGTGTGAGATGGT | ovarian serous cystadenocarcinoma |
| 1008 | TCTCACCCCAACTCTGCCCCAGA | ovarian serous cystadenocarcinoma |
| 1009 | TCTCGTCGCGCGCGCGTCCGT | ovarian serous cystadenocarcinoma |
| 1010 | TCTCGTCTGATCTCGGACGCTAAG | ovarian serous cystadenocarcinoma |
| 1011 | TCTGACTGCCTGTGCTGAG | ovarian serous cystadenocarcinoma |
| 1012 | TCTGTGCGAAGGTCCGTGGA | ovarian serous cystadenocarcinoma |
| 1013 | TCTTATCTCGGAAGC | ovarian serous cystadenocarcinoma |
| 1014 | TGAACGATCGTCAGGGTTTT | ovarian serous cystadenocarcinoma |
| 1015 | TGAAGCAGCCATGATCTG | ovarian serous cystadenocarcinoma |
| 1016 | TGCCCTGCGGCCCCGGCTCTGA | ovarian serous cystadenocarcinoma |
| 1017 | TGCCGGTCGCTCGCCCTCTA | ovarian serous cystadenocarcinoma |
| 1018 | TGGACAGACACAGGCCTGAGG | ovarian serous cystadenocarcinoma |
| 1019 | TGGACTCGAGGGCTGGGCG | ovarian serous cystadenocarcinoma |
| 1020 | TGGAGCGTCGGGCCCCTACCCGGCCGTCGC | ovarian serous cystadenocarcinoma |
| 1021 | TGGAGGTGCTGAACTGTCTGAGCCTG | ovarian serous cystadenocarcinoma |
| 1022 | TGGAGTGATTTGTCTGG | ovarian serous cystadenocarcinoma |
| 1023 | TGGATGTCCGGGGCTGCA | ovarian serous cystadenocarcinoma |
| 1024 | TGGCGTTCGCCGGGCTGGAGC | ovarian serous cystadenocarcinoma |
| 1025 | TGGCTGCTGGGACTCAAG | ovarian serous cystadenocarcinoma |
| 1026 | TGGGTCGGTCGAGCTGGGGCGC | ovarian serous cystadenocarcinoma |
| 1027 | TGGTCTGGAACTGAACCTGCAGT | ovarian serous cystadenocarcinoma |
| 1028 | TGGTCTGTGGAGCTCGGCGGCG | ovarian serous cystadenocarcinoma |
| 1029 | TGGTGGCGGGGAGCGGT | ovarian serous cystadenocarcinoma |
| 1030 | TGTGAGAACGCGGTCTGAGTGGT | ovarian serous cystadenocarcinoma |
| 1031 | TGTGATGCGAACTGGAGTCTGAGCCT | ovarian serous cystadenocarcinoma |
| 1032 | TGTGATTTCTGCCCAGTGC | ovarian serous cystadenocarcinoma |
| 1033 | TGTGTAGGATTCTGACCCAC | ovarian serous cystadenocarcinoma |
| 1034 | TGTTGGTTTTCGGCACTGAG | ovarian serous cystadenocarcinoma |
| 1035 | TGTTTACCCTCTTACTTTC | ovarian serous cystadenocarcinoma |
| 1036 | TTAATAGGAACAGTCGG | ovarian serous cystadenocarcinoma |
| 1037 | TTATGCCTGAACGCCTCTA | ovarian serous cystadenocarcinoma |
| 1038 | TTCAATACGGGACTGATG | ovarian serous cystadenocarcinoma |
| 1039 | TTCAGATTTTTGGATTAAGGACGTTCAACT | ovarian serous cystadenocarcinoma |
| 1040 | TTCCACGCTCTTCCCTGCCT | ovarian serous cystadenocarcinoma |
| 1041 | TTCCGATACCGAACGAGACTCTGGCAT | ovarian serous cystadenocarcinoma |
| 1042 | TTCGAACTTGACTATCT | ovarian serous cystadenocarcinoma |
| 1043 | TTCGGATGGGGCTGTTCTGCG | ovarian serous cystadenocarcinoma |
| 1044 | TTCGGTCGGGCTGGG | ovarian serous cystadenocarcinoma |
| 1045 | TTCTTTCGCTCCTATCTGATG | ovarian serous cystadenocarcinoma |
| 1046 | TTGAGGTCCGGGGCAGAG | ovarian serous cystadenocarcinoma |
| 1047 | TTGATCACTCGGCTCG | ovarian serous cystadenocarcinoma |
| 1048 | TTGCCGGTCGCTCGCCCTCTT | ovarian serous cystadenocarcinoma |
| 1049 | TTGGGGTTGTGGGACAGT | ovarian serous cystadenocarcinoma |
| 1050 | TTGGGTTTTAAGCAGGAGGT | ovarian serous cystadenocarcinoma |
| 1051 | TTGGTCCGCGAGGTCCCGGGT | ovarian serous cystadenocarcinoma |
| 1052 | TTGTCTGGGCCCGGAGTAGG | ovarian serous cystadenocarcinoma |
| 1053 | TTGTTTGGGATTGCAG | ovarian serous cystadenocarcinoma |
| 1054 | TTTCAGAGTAGTTGGGGTTG | ovarian serous cystadenocarcinoma |
| 1055 | TTTGCAGGACCAGGGCCGG | ovarian serous cystadenocarcinoma |
| 1056 | TTTGGCAATGGTAGACCTCCCACT | ovarian serous cystadenocarcinoma |
| 1057 | AACATCGGATTTGGGGAACGC | mesothelioma, pancreatic adenocarcinoma |
| 1058 | CCGCTCCCCGGCGCCGCTCGTGGG | adrenocortical carcinoma, pancreatic |
| | | adenocarcinoma |
| 1059 | GGAGCGCGCCGCGGCTGGACGAG | adrenocortical carcinoma, pancreatic |
| | | adenocarcinoma |
| 1060 | ATGTTTGTACTCTGGCCGCT | brain lower grade glioma, pancreatic |
| | | adenocarcinoma, uveal melanoma |
| 1061 | AGGGGGGGAGTTTGACTGGGGCG | cervical squamous cell carcinoma and |
| | | endocervical adenocarcinoma, pancreatic |
| | | adenocarcinoma |
| 1062 | CACTGTCCCTACCTACTCTC | cervical squamous cell carcinoma and |
| | | endocervical adenocarcinoma, pancreatic |
| | | adenocarcinoma |
| 1063 | CGGACCAGGGGAATCCGCCTGTT | cervical squamous cell carcinoma and |
| | | endocervical adenocarcinoma, pancreatic |
| | | adenocarcinoma |
| 1064 | GTCGGGGTTTCGTACGTCG | cervical squamous cell carcinoma and |
| | | endocervical adenocarcinoma, pancreatic |
| | | adenocarcinoma, uterine corpus endometrial |
| | | carcinoma |
| 1065 | GTCTGGGTTTCGTACGTAGCA | cervical squamous cell carcinoma and |
| | | endocervical adenocarcinoma, pancreatic |
| | | adenocarcinoma |
| 1066 | ACCATGACCGAGACGTGTGGAA | cholangiocarcinoma, pancreatic |
| | | adenocarcinoma |
| 1067 | CGAGCCGTCGGGCCCGGGGGAGGTT | cholangiocarcinoma, pancreatic |
| | | adenocarcinoma |
| 1068 | GATGGCGCTGGAGCGTCGGGCCCCT | cholangiocarcinoma, pancreatic |
| | | adenocarcinoma |
| 1069 | CGAGAGAGGGGCCCGTGCCTTGG | head and neck squamous cell carcinoma, |
| | | pancreatic adenocarcinoma |
| 1070 | AAAACCAGTCGATGGGCTGTCTT | pancreatic adenocarcinoma |
| 1071 | AAAACCTTGCCAGGGATTTCC | pancreatic adenocarcinoma |
| 1072 | AAAATTCAACGCTGTCTGTGA | pancreatic adenocarcinoma |
| 1073 | AAACCTCTGCAGGCAAATGTG | pancreatic adenocarcinoma |
| 1074 | AACACGTGTGGAAAACTAATGACTGAGC | pancreatic adenocarcinoma |
| 1075 | AACATTCAACGCTGTCCGCGAGT | pancreatic adenocarcinoma |
| 1076 | AAGACATTTCCAGGGATTACC | pancreatic adenocarcinoma |
| 1077 | AAGACATTTCCAGGGATTTCC | pancreatic adenocarcinoma |
| 1078 | AAGACCTTGCCAGGGATTACCA | pancreatic adenocarcinoma |
| 1079 | AAGACCTTGCCAGGGATTTC | pancreatic adenocarcinoma |
| 1080 | AAGACCTTGCCAGGGATTTCCAA | pancreatic adenocarcinoma |
| 1081 | AAGCCGTAAATCCGATCTTGT | pancreatic adenocarcinoma |
| 1082 | AAGCTGCCAGTTGAAGCCTTGA | pancreatic adenocarcinoma |
| 1083 | AAGCTGCCAGTTTAAGCCGTGT | pancreatic adenocarcinoma |
| 1084 | AAGGTGCCAGCTGAAGCACTGT | pancreatic adenocarcinoma |
| 1085 | AAGGTGCCAGTTTAAGAAATGT | pancreatic adenocarcinoma |
| 1086 | AATCTATCAGACTGATGTTGA | pancreatic adenocarcinoma |
| 1087 | ACACTGTATACCCGAATTTGT | pancreatic adenocarcinoma |
| 1088 | ACAGCAGTCTGCCCATTGGTT | pancreatic adenocarcinoma |
| 1089 | ACAGTGTATATCCGAATTTGTGT | pancreatic adenocarcinoma |
| 1090 | ACCATGACCGAGACGTGTGGAAA | pancreatic adenocarcinoma |
| 1091 | ACCGTAGTCAGCACATTGGTT | pancreatic adenocarcinoma |
| 1092 | ACCGTAGTCTGCACATTCGTT | pancreatic adenocarcinoma |
| 1093 | ACCGTATTCTGCACATTGGTA | pancreatic adenocarcinoma |
| 1094 | ACCGTATTCTGCACATTGTTT | pancreatic adenocarcinoma |
| 1095 | ACCTTATTCTGCACATTGCT | pancreatic adenocarcinoma |
| 1096 | ACCTTTGTCTGCACATTGGTTA | pancreatic adenocarcinoma |
| 1097 | ACGCTGTAAATCCGAATTTG | pancreatic adenocarcinoma |
| 1098 | ACGCTGTACATCCGAATTTGTG | pancreatic adenocarcinoma |
| 1099 | ACGCTGTATATCCGAATTTG | pancreatic adenocarcinoma |
| 1100 | ACTACTGACCGTTGACTGTTCC | pancreatic adenocarcinoma |
| 1101 | ACTAGATTGTGAGCTCATCGAG | pancreatic adenocarcinoma |
| 1102 | ACTCTGTATATCCGAATTTGTTT | pancreatic adenocarcinoma |
| 1103 | ACTGCACTTGACCGCTCTGA | pancreatic adenocarcinoma |
| 1104 | ACTTTCGTCTGCACATTGGT | pancreatic adenocarcinoma |
| 1105 | AGAGCCTTGCCAGGGATTTCCAG | pancreatic adenocarcinoma |
| 1106 | AGAGTATTCTGCACATTGGT | pancreatic adenocarcinoma |
| 1107 | AGAGTGGGAGTTGAAGAACTGT | pancreatic adenocarcinoma |
| 1108 | AGATTGGACCAGACCCTGA | pancreatic adenocarcinoma |
| 1109 | AGCAGCATTGCACAGGGCTCTGA | pancreatic adenocarcinoma |
| 1110 | AGCGTCTCAGACTGATGTTGA | pancreatic adenocarcinoma |
| 1111 | AGGGCACTGGTAGAATTCGCT | pancreatic adenocarcinoma |
| 1112 | AGGGCACTTGTAGAATTCACT | pancreatic adenocarcinoma |
| 1113 | AGTGCTGGGCTGTAGTGCGCT | pancreatic adenocarcinoma |
| 1114 | ATAACATGGCCAGGGATTTCCA | pancreatic adenocarcinoma |
| 1115 | ATAGCCATTGTCTGCTGGGTTT | pancreatic adenocarcinoma |
| 1116 | ATCAGAATTGTACAGGTCTCTGA | pancreatic adenocarcinoma |
| 1117 | ATCAGCATTGTCCAGGTCTATGA | pancreatic adenocarcinoma |
| 1118 | ATCATATCAGACTGATGTTGA | pancreatic adenocarcinoma |
| 1119 | ATCTGCCATTTGAAGACCTGT | pancreatic adenocarcinoma |
| 1120 | ATGACATTTCCAGGGATTTCC | pancreatic adenocarcinoma |
| 1121 | ATGCTGCCAGTTGAAGTAGTGT | pancreatic adenocarcinoma |
| 1122 | ATGGACATTGTCTGCTGGGTT | pancreatic adenocarcinoma |
| 1123 | ATGGCACTTGTAGAATTCAATG | pancreatic adenocarcinoma |
| 1124 | ATGTCTTTTTGGAAATAGGA | pancreatic adenocarcinoma |
| 1125 | ATTCTGCATCAGGAACTGATTGT | pancreatic adenocarcinoma |
| 1126 | ATTGGGTCCGAGTGTTGTGGGTTATTGTT | pancreatic adenocarcinoma |
| 1127 | CAACATGATGGAACTGAGGCTCTGAGG | pancreatic adenocarcinoma |
| 1128 | CAAGACCAGTCGATGGGCTGA | pancreatic adenocarcinoma |
| 1129 | CACAGTTCTGCTCGTGCAGGTAG | pancreatic adenocarcinoma |
| 1130 | CACCACCAGTCGATGGGCTATT | pancreatic adenocarcinoma |
| 1131 | CACCCCCGGTCGATGGGCTGT | pancreatic adenocarcinoma |
| 1132 | CACGTCACTAGTGGTTCCGTTTAGT | pancreatic adenocarcinoma |
| 1133 | CAGACCTATGAATTGACAGTCA | pancreatic adenocarcinoma |
| 1134 | CAGAGTGGTCTCTGGGATTATG | pancreatic adenocarcinoma |
| 1135 | CAGCGCCTGTGACAGCGGCTGA | pancreatic adenocarcinoma |
| 1136 | CAGCGCGTTTGACAGCGGCTGA | pancreatic adenocarcinoma |
| 1137 | CAGGAGTGTGCACATTGGTT | pancreatic adenocarcinoma |
| 1138 | CAGTTCGGAGTTAGCTCAAGCGG | pancreatic adenocarcinoma |
| 1139 | CATGGTACTTGTCTCGGTCTG | pancreatic adenocarcinoma |
| 1140 | CCAAGTGTTTAGACTACCTGTTC | pancreatic adenocarcinoma |
| 1141 | CCCACCCTCAGATCAGACGT | pancreatic adenocarcinoma |
| 1142 | CCCAGTTTTCAGACTAGCTGTT | pancreatic adenocarcinoma |
| 1143 | CCCATCTGACTCCTTTTCTTCTAGA | pancreatic adenocarcinoma |
| 1144 | CCCCGTCTCGTCGCGCGCGCGTC | pancreatic adenocarcinoma |
| 1145 | CCCTGGTCGGAGTTAGCTCAAGC | pancreatic adenocarcinoma, skin cutaneous |
| | | melanoma |
| 1146 | CCGAGTGTTCAGACTATCTGTTC | pancreatic adenocarcinoma |
| 1147 | CCGCGGGGCCCCGGCGGCGGGGGCACG | pancreatic adenocarcinoma |
| 1148 | CCTAGTTTTCAGACTAGCTGTTC | pancreatic adenocarcinoma |
| 1149 | CCTCGGACTACCGGGTGCTGTAGGCTT | pancreatic adenocarcinoma |
| 1150 | CCTGAGCCAGCAGATCGAGT | pancreatic adenocarcinoma |
| 1151 | CGAGCACCAGCTGAAATCGGTT | pancreatic adenocarcinoma |
| 1152 | CGAGCCCCATCTGAAATCGG | pancreatic adenocarcinoma |
| 1153 | CGGGACGCGAGCCGGGCCCTT | pancreatic adenocarcinoma |
| 1154 | CGTGCTGTCGGATGTTTACAG | pancreatic adenocarcinoma |
| 1155 | CTATCTCCCCCGCTCCCCGTC | pancreatic adenocarcinoma |
| 1156 | CTATCTCTCTCCCCCGCTCC | pancreatic adenocarcinoma |
| 1157 | CTCTGCGGGTGACAGCGGCTGA | pancreatic adenocarcinoma |
| 1158 | CTCTGCGTGTGACAGCGGCTGAG | pancreatic adenocarcinoma |
| 1159 | CTGCCCTGAAGCTCCTTGAGGT | pancreatic adenocarcinoma |
| 1160 | CTGGCTTAGCGTCGCT | pancreatic adenocarcinoma |
| 1161 | CTGGGTCGGTCGGGCTGGGGCGG | pancreatic adenocarcinoma |
| 1162 | CTTATCATATTGTATTGTGATT | pancreatic adenocarcinoma |
| 1163 | CTTTCATGCGGATGTTTAAAG | pancreatic adenocarcinoma |
| 1164 | GCAACCATCCTTGACTGGCAGCT | pancreatic adenocarcinoma |
| 1165 | GCATCAGTGGCCTAATGGA | pancreatic adenocarcinoma |
| 1166 | GCGCGCTCCGCGGCTGGACGAG | pancreatic adenocarcinoma |
| 1167 | GCGCGCTCGCCGGCCGAGGTG | pancreatic adenocarcinoma |
| 1168 | GCGGGTGGTGTGCGTCGG | pancreatic adenocarcinoma |
| 1169 | GGACGCCGGTGAAATACCACTAC | pancreatic adenocarcinoma |
| 1170 | GGACGCCGGTGAAATACCACTACTCTGA | pancreatic adenocarcinoma |
| 1171 | GGAGATCAGACTGATGTTGA | pancreatic adenocarcinoma |
| 1172 | GGAGCCGGTGAAATACCACTACTCTGA | pancreatic adenocarcinoma |
| 1173 | GGAGCTTATCAGACTGATCTTGA | pancreatic adenocarcinoma |
| 1174 | GGAGGAAGGAGGTTGTGTGG | pancreatic adenocarcinoma |
| 1175 | GGAGGAAGTAGGTTGTGTGGTT | pancreatic adenocarcinoma |
| 1176 | GGCCGCGCCGTCGGGCCCGGGGGAG | pancreatic adenocarcinoma |
| 1177 | GGCCTCAGGTACCATGACCGAG | pancreatic adenocarcinoma |
| 1178 | GGCGTTCTGGGCTGTAGTGCGCT | pancreatic adenocarcinoma |
| 1179 | GGGCGCTTTGCCTAATGGA | pancreatic adenocarcinoma |
| 1180 | GGGGGATTATGACTGAACGCCT | pancreatic adenocarcinoma |
| 1181 | GTAGCTTATCAGACTGATCTTG | pancreatic adenocarcinoma |
| 1182 | GTAGCTTATCATACTGCTGTTGA | pancreatic adenocarcinoma, uveal melanoma |
| 1183 | GTATCTTATCCGACTGATGTTGA | pancreatic adenocarcinoma |
| 1184 | GTCCCGTCCTGTGATAGCTGA | pancreatic adenocarcinoma |
| 1185 | GTCGTCTTGGCCGAGTGGTTAAGG | pancreatic adenocarcinoma |
| 1186 | GTGACATTTTTAGGACCATTAGA | pancreatic adenocarcinoma |
| 1187 | GTGCCATTGGCGCAATGGC | pancreatic adenocarcinoma |
| 1188 | GTGCGCTCGCCGCGGCTGGACG | pancreatic adenocarcinoma |
| 1189 | GTGTGCGCGCCGCGGCTGGACG | pancreatic adenocarcinoma |
| 1190 | GTTACCATCCTTGACTGGCAGC | pancreatic adenocarcinoma |
| 1191 | GTTTTCGGAACTGCGGCCATG | pancreatic adenocarcinoma, rectum |
| | | adenocarcinoma |
| 1192 | TAAAGCAGGCACAGACAGGCAGA | pancreatic adenocarcinoma |
| 1193 | TAAAGTACTGTGATAACTGGAC | pancreatic adenocarcinoma |
| 1194 | TAAAGTACTGTTATAACTTC | pancreatic adenocarcinoma |
| 1195 | TAAAGTAGTATGCACATTGGTT | pancreatic adenocarcinoma |
| 1196 | TAACACAGTGTAGAACCACGGAC | pancreatic adenocarcinoma |
| 1197 | TAACTTATCACACTGATGTTG | pancreatic adenocarcinoma |
| 1198 | TAAGATTAAGCACTGTCGTT | pancreatic adenocarcinoma |
| 1199 | TAAGCTCAGCTGGCAACTGTGAG | pancreatic adenocarcinoma |
| 1200 | TAAGCTTAAGCACTGTCGCA | pancreatic adenocarcinoma |
| 1201 | TAAGGTATTAGGTTGTCTTGTT | pancreatic adenocarcinoma |
| 1202 | TAAGGTGGTAGGTTGTCTAGTT | pancreatic adenocarcinoma |
| 1203 | TAAGTTGTCAGACTGATGTTGAA | pancreatic adenocarcinoma |
| 1204 | TAAGTTGTCAGACTGTTGTTG | pancreatic adenocarcinoma |
| 1205 | TACAATACTGTGATAAATGAAC | pancreatic adenocarcinoma |
| 1206 | TACACCGTTGCTAAGTTCTGC | pancreatic adenocarcinoma |
| 1207 | TACAGTATTCTGCACATTTGT | pancreatic adenocarcinoma |
| 1208 | TACCCTATAGATCCGATTTTG | pancreatic adenocarcinoma |
| 1209 | TACCCTGTAGCTCAGACTTTGT | pancreatic adenocarcinoma |
| 1210 | TACCCTTAAGAACTGTAGCTC | pancreatic adenocarcinoma |
| 1211 | TACCCTTTAGCTCAGAATTTGT | pancreatic adenocarcinoma |
| 1212 | TACCTTGTCAGACTGAAGTTG | pancreatic adenocarcinoma |
| 1213 | TACGATGAATCACTGTAGCT | pancreatic adenocarcinoma |
| 1214 | TACTTTATCTGACTGATGTTGA | pancreatic adenocarcinoma |
| 1215 | TACTTTGTCAGACTGAAGTTGA | pancreatic adenocarcinoma |
| 1216 | TAGACCGTGGCTAAGTTCTG | pancreatic adenocarcinoma |
| 1217 | TAGCATCATCTTAAATCGTTT | pancreatic adenocarcinoma |
| 1218 | TAGCCTAACACACTGATGTTGA | pancreatic adenocarcinoma |
| 1219 | TAGCCTATAGATCCGATTTTGA | pancreatic adenocarcinoma |
| 1220 | TAGCGTATTAGGTTGTCTTGTT | pancreatic adenocarcinoma |
| 1221 | TAGCGTGGTAGGTTGTATCGTT | pancreatic adenocarcinoma |
| 1222 | TAGCTCAGTTCAGCAGGAAGAG | pancreatic adenocarcinoma |
| 1223 | TAGCTTAGAAGACTGATATTGA | pancreatic adenocarcinoma |
| 1224 | TAGCTTAGCACACTGATGTTG | pancreatic adenocarcinoma |
| 1225 | TAGCTTAGCAGACTGTAGATGAC | pancreatic adenocarcinoma |
| 1226 | TAGCTTAGCAGACTTATGTTG | pancreatic adenocarcinoma |
| 1227 | TAGCTTAGCAGTCTGATGTTGA | pancreatic adenocarcinoma |
| 1228 | TAGCTTATCAGCCTAATGTTGC | pancreatic adenocarcinoma |
| 1229 | TAGCTTATCAGCCTTCTGTTGA | pancreatic adenocarcinoma |
| 1230 | TAGCTTATCAGGGTTATGTTGA | pancreatic adenocarcinoma |
| 1231 | TAGCTTATCAGTCTGACGTTGA | pancreatic adenocarcinoma |
| 1232 | TAGCTTATCTGACTGAGGTTGAC | pancreatic adenocarcinoma |
| 1233 | TAGCTTATGAGACTGATGGTGAC | pancreatic adenocarcinoma |
| 1234 | TAGCTTGTCAGACTGAAGTT | pancreatic adenocarcinoma |
| 1235 | TAGCTTGTCAGACTGATATTGA | pancreatic adenocarcinoma |
| 1236 | TAGCTTGTCAGCCTGATGGTGA | pancreatic adenocarcinoma |
| 1237 | TAGCTTGTCAGGCTGAAGCTGA | pancreatic adenocarcinoma |
| 1238 | TAGGGTAGTAGTTTGTATAGT | pancreatic adenocarcinoma |
| 1239 | TAGGGTAGTAGTTTGTCCAGTT | pancreatic adenocarcinoma |
| 1240 | TAGGTTATCCGACTGATGTTGC | pancreatic adenocarcinoma |
| 1241 | TAGGTTATCCGCCTGATGTTGA | pancreatic adenocarcinoma |
| 1242 | TAGTGTAGTAGGTTGTGTTG | pancreatic adenocarcinoma |
| 1243 | TAGTGTATTAGGTTGTGTGG | pancreatic adenocarcinoma |
| 1244 | TAGTTTAGCAGACTGAAGATGA | pancreatic adenocarcinoma |
| 1245 | TAGTTTATCAGATTGATGTTG | pancreatic adenocarcinoma |
| 1246 | TATACTTAAGCACTGTAGATCT | pancreatic adenocarcinoma |
| 1247 | TATAGTAATCTGCACATTGGTT | pancreatic adenocarcinoma |
| 1248 | TATAGTAGTAGGTTGTGGGG | pancreatic adenocarcinoma |
| 1249 | TATAGTATTAGTTTGTATAGTT | pancreatic adenocarcinoma |
| 1250 | TATATACCTCTTCGACTGGAAG | pancreatic adenocarcinoma |
| 1251 | TATCCTGTAGATCCGAGTTTG | pancreatic adenocarcinoma |
| 1252 | TATCCTTTAGAACAGACTTTGT | pancreatic adenocarcinoma |
| 1253 | TATCCTTTAGATCAGACTTTGT | pancreatic adenocarcinoma |
| 1254 | TATCCTTTAGATCCGAATTTGTT | pancreatic adenocarcinoma |
| 1255 | TATGGTATTAGGTTGTCTCGTT | pancreatic adenocarcinoma |
| 1256 | TCAAAGTTTCCGGGTTCGTGTCCCGGCGG | pancreatic adenocarcinoma |
| 1257 | TCAGTGCGCTAGAGAACTCTGT | pancreatic adenocarcinoma |
| 1258 | TCCCTGATACCCTAACCTGTGA | pancreatic adenocarcinoma |
| 1259 | TCCTGAAGTAGGTTGTATAGTT | pancreatic adenocarcinoma |
| 1260 | TCGAATTAAGCACTGTAGCTCA | pancreatic adenocarcinoma |
| 1261 | TCGTTGCACGACAGAACTTTG | pancreatic adenocarcinoma |
| 1262 | TCTATGCACCACAGAACTTTGT | pancreatic adenocarcinoma |
| 1263 | TCTGTGCACGACAGAACTTTG | pancreatic adenocarcinoma |
| 1264 | TCTGTGCACTCCAGAAATTTGT | pancreatic adenocarcinoma |
| 1265 | TGAACAGTTGCTAAGTTCTG | pancreatic adenocarcinoma |
| 1266 | TGAGATGAAGCACTGTGTGG | pancreatic adenocarcinoma |
| 1267 | TGAGATGACACACTGTTGTTCT | pancreatic adenocarcinoma |
| 1268 | TGAGATGGAGCACTTTCGCTCT | pancreatic adenocarcinoma |
| 1269 | TGAGATTAAGAACTGTCGTTC | pancreatic adenocarcinoma |
| 1270 | TGAGCAGTTGCTAAGTTCCG | pancreatic adenocarcinoma |
| 1271 | TGAGCAGTTGCTAAGTTCTG | pancreatic adenocarcinoma |
| 1272 | TGAGCCGAATCTCCAGGGGTAA | pancreatic adenocarcinoma |
| 1273 | TGAGCCGTGGCTAAGTTCTG | pancreatic adenocarcinoma |
| 1274 | TGAGCTTAAGCCCTGTAGGT | pancreatic adenocarcinoma |
| 1275 | TGAGGTAATAGGTTGTCTTGTT | pancreatic adenocarcinoma |
| 1276 | TGAGGTAGTAGGTTGTGCGGCT | pancreatic adenocarcinoma |
| 1277 | TGAGGTATTAGATTGTCTCG | pancreatic adenocarcinoma |
| 1278 | TGAGGTATTAGGTTGTCTTGTTT | pancreatic adenocarcinoma |
| 1279 | TGAGGTATTAGGTTGTTTTGTTA | pancreatic adenocarcinoma |
| 1280 | TGAGGTCGTTCGTCTTATAGTT | pancreatic adenocarcinoma |
| 1281 | TGAGTTAGCAGACTTATGTTGA | pancreatic adenocarcinoma |
| 1282 | TGAGTTATAAGACTGATGGTGA | pancreatic adenocarcinoma |
| 1283 | TGAGTTATCAGACTTTTGTTGA | pancreatic adenocarcinoma |
| 1284 | TGAGTTGTCAGAATGATGTTGA | pancreatic adenocarcinoma |
| 1285 | TGAGTTGTCAGACTGACGTTG | pancreatic adenocarcinoma |
| 1286 | TGATATTAAGAACTGTCGCT | pancreatic adenocarcinoma |
| 1287 | TGCACAGTTGCTAAGTTCTG | pancreatic adenocarcinoma |
| 1288 | TGCCCTGTGGACTCAGTTCTGGG | pancreatic adenocarcinoma |
| 1289 | TGCCTTATCAGACGGATGTTGA | pancreatic adenocarcinoma |
| 1290 | TGCGATATTAGGTTGTGTGG | pancreatic adenocarcinoma |
| 1291 | TGCGGTAGTCGGTTGTGTGGTTT | pancreatic adenocarcinoma |
| 1292 | TGCGGTATTAGATTGTCTGGTT | pancreatic adenocarcinoma |
| 1293 | TGCTATGCCAACATATTGCCCTC | pancreatic adenocarcinoma |
| 1294 | TGCTATTAAGCACTGTGGGTCT | pancreatic adenocarcinoma |
| 1295 | TGCTCTTCAGCACTGTAGCTA | pancreatic adenocarcinoma |
| 1296 | TGCTGTAATAGGTTGTCTCGTT | pancreatic adenocarcinoma |
| 1297 | TGCTGTAGTAGTTTGTCCAGT | pancreatic adenocarcinoma |
| 1298 | TGGCTTATCAGACGGATGGTGA | pancreatic adenocarcinoma |
| 1299 | TGGCTTATCAGCCTGATGCTGA | pancreatic adenocarcinoma |
| 1300 | TGTAACATCAACCCCATGTGGA | pancreatic adenocarcinoma |
| 1301 | TGTAGCACTGGTAGAACTCA | pancreatic adenocarcinoma |
| 1302 | TTAACAGTTGCTAAGTTCCG | pancreatic adenocarcinoma |
| 1303 | TTAGGTAGTAGGTCGTGTGG | pancreatic adenocarcinoma |
| 1304 | TTAGGTAGTTGGTTGTATAG | pancreatic adenocarcinoma |
| 1305 | TTATCTTCAGCACTGTCGCT | pancreatic adenocarcinoma |
| 1306 | TTCATAGTATTTACCAACCAGT | pancreatic adenocarcinoma |
| 1307 | TTCATGCTAATCGTGATACGGGT | pancreatic adenocarcinoma |
| 1308 | TTCCATAACAGACTGATGTTGA | pancreatic adenocarcinoma |
| 1309 | TTCCCTATGTATTGACAGCCAG | pancreatic adenocarcinoma |
| 1310 | TTCCGTATTAGGTTGTATAG | pancreatic adenocarcinoma |
| 1311 | TTCCTAATCAGACTGATGTTGA | pancreatic adenocarcinoma |
| 1312 | TTCGGTATTAGATTGTATCGTT | pancreatic adenocarcinoma |
| 1313 | TTCTACCCACGTCTGCTCTGA | pancreatic adenocarcinoma |
| 1314 | TTCTATAAAGCACTGTCGTTCT | pancreatic adenocarcinoma |
| 1315 | TTCTATATTAGGTTGTCTCGTT | pancreatic adenocarcinoma |
| 1316 | TTCTCTATGAATTGACACCCAG | pancreatic adenocarcinoma |
| 1317 | TTCTGGGGTAGGTTGTGTGGTT | pancreatic adenocarcinoma |
| 1318 | TTCTGTAATAGGTTGTCTTGTT | pancreatic adenocarcinoma |
| 1319 | TTCTGTAATAGTTTGTATAGT | pancreatic adenocarcinoma |
| 1320 | TTCTGTAGTAGTTTGTATCGTT | pancreatic adenocarcinoma |
| 1321 | TTCTGTAGTAGTTTGTCTAGT | pancreatic adenocarcinoma |
| 1322 | TTCTGTAGTTGGTTGTATGGTT | pancreatic adenocarcinoma |
| 1323 | TTCTGTATTAGATTGTATAGA | pancreatic adenocarcinoma |
| 1324 | TTCTGTATTAGATTGTCTGGTT | pancreatic adenocarcinoma |
| 1325 | TTCTGTATTAGGTTGTCTCG | pancreatic adenocarcinoma |
| 1326 | TTCTGTATTAGGTTGTCTCGTTT | pancreatic adenocarcinoma |
| 1327 | TTCTGTATTAGGTTGTGTCGTT | pancreatic adenocarcinoma |
| 1328 | TTCTGTATTAGGTTGTGTTGTTT | pancreatic adenocarcinoma |
| 1329 | TTCTGTATTAGGTTGTTTTGTT | pancreatic adenocarcinoma |
| 1330 | TTCTGTATTAGTTTGTCTTGTT | pancreatic adenocarcinoma |
| 1331 | TTCTGTGTTAGGTTGTCTCGTT | pancreatic adenocarcinoma |
| 1332 | TTCTTTAGCAGACTGAAGATGA | pancreatic adenocarcinoma |
| 1333 | TTCTTTAGCAGACTGAAGTTGA | pancreatic adenocarcinoma |
| 1334 | TTCTTTATCAAACTGATGTTG | pancreatic adenocarcinoma |
| 1335 | TTCTTTATCACACTGTTGTTGA | pancreatic adenocarcinoma |
| 1336 | TTCTTTATTACACTGATGTTGA | pancreatic adenocarcinoma |
| 1337 | TTCTTTATTAGTTTGTGTGGTT | pancreatic adenocarcinoma |
| 1338 | TTCTTTGTCACACTGATGTTGA | pancreatic adenocarcinoma |
| 1339 | TTGCATGTCAGACTGAAGCTGA | pancreatic adenocarcinoma |
| 1340 | TTGCTCATTTCAGCAGGAGCAG | pancreatic adenocarcinoma |
| 1341 | TTGCTTATCAGACGGATGTTG | pancreatic adenocarcinoma |
| 1342 | TTGGAGACTATGGCACGTAG | pancreatic adenocarcinoma |
| 1343 | TTGGCTTAAGCACTGTAGA | pancreatic adenocarcinoma |
| 1344 | TTGGCTTAAGCACTGTAGGTCT | pancreatic adenocarcinoma |
| 1345 | TTGGCTTAAGCACTGTGGGTCT | pancreatic adenocarcinoma |
| 1346 | TTGGGTAGGAGGTTGTATAGA | pancreatic adenocarcinoma |
| 1347 | TTGGGTAGTACGTTGTATAGT | pancreatic adenocarcinoma |
| 1348 | TTGGGTAGTAGATTGTCTGGTT | pancreatic adenocarcinoma |
| 1349 | TTGGTAATCAGACTGATGTTGA | pancreatic adenocarcinoma |
| 1350 | TTGGTAATCAGACTGATGTTGA | pancreatic adenocarcinoma |
| 1351 | TTGGTCAGTTCAGCAGGCACAG | pancreatic adenocarcinoma |
| 1352 | TTGGTCATCAGACTGATGTTGA | pancreatic adenocarcinoma |
| 1353 | TTGGTTAGCAGACTTATGTTGA | pancreatic adenocarcinoma |
| 1354 | TTGGTTATCAGACTGATGTTGG | pancreatic adenocarcinoma |
| 1355 | TTGGTTATTAGGTTGTGTGGTT | pancreatic adenocarcinoma |
| 1356 | TTGGTTGTCAGACTGATGTTG | pancreatic adenocarcinoma |
| 1357 | TTGTGTAGTAGGTTGTCTGGT | pancreatic adenocarcinoma |
| 1358 | TTGTGTATTAGGTTGTATAGA | pancreatic adenocarcinoma |
| 1359 | TTGTGTATTAGGTTGTGTGGTTA | pancreatic adenocarcinoma |
| 1360 | TTGTTCATTTCAGCAGGAACAG | pancreatic adenocarcinoma |
| 1361 | TTGTTTATCAGGCTGATGTTG | pancreatic adenocarcinoma |
| 1362 | TTTAACCATCCTCGACTGGGAGCT | pancreatic adenocarcinoma |
| 1363 | TTTACACCTCCTACACTCTCAGCT | pancreatic adenocarcinoma |
| 1364 | TTTGCTGAAGCACTGTAGTTCT | pancreatic adenocarcinoma |
| 1365 | TTTGCTGGAGCACTGTAGCT | pancreatic adenocarcinoma |
| 1366 | TTTGGTAGTCTGCACATTGGT | pancreatic adenocarcinoma |
| 1367 | AAAAGTGCTTACCGTGCAGGTAGT | prostate adenocarcinoma |
| 1368 | AAGCTGCCAGCTCCATAACTGT | prostate adenocarcinoma |
| 1369 | AAGCTTCCAGCTCCATAACTGT | prostate adenocarcinoma |
| 1370 | AATTGCACGGTGTCCATCTGTA | prostate adenocarcinoma |
| 1371 | AATTTTCCAGTACCGTGTTGG | prostate adenocarcinoma |
| 1372 | ACACCATGATGGCACTGAGGATCTGCGG | prostate adenocarcinoma |
| 1373 | AGGAGACTCTGGACGCGAGCCG | prostate adenocarcinoma |
| 1374 | ATGGAACTGAGGCTCTGAGGAA | prostate adenocarcinoma |
| 1375 | CAAAGTGCTCATCGTTCAGGTAG | prostate adenocarcinoma |
| 1376 | CAGAATGCTGACTAGTAGACTA | prostate adenocarcinoma |
| 1377 | CAGCTGCCAGGTGCATAACTGT | prostate adenocarcinoma |
| 1378 | CAGCTTCCAGCTGCATAGCTGT | prostate adenocarcinoma |
| 1379 | CCACCATGATGGAACTGAG | prostate adenocarcinoma |
| 1380 | CGTAGGGACCTGTATGAATGGC | prostate adenocarcinoma |
| 1381 | CTCGTACGACCCTTATCGGTGG | prostate adenocarcinoma |
| 1382 | CTGGATGATGCTCAGCAAATGCTGACTGA | prostate adenocarcinoma |
| 1383 | GAGGGTGATGCGAACTGGAGTCTGNGC | prostate adenocarcinoma |
| 1384 | GCGGTCGGCGGGCGGCGGGGGC | prostate adenocarcinoma |
| 1385 | GCTTGTGATGACTTACATGTA | prostate adenocarcinoma |
| 1386 | GGATTCCTGGCACTACTGTTCT | prostate adenocarcinoma |
| 1387 | GTACAGTACTGTTATAACTTAA | prostate adenocarcinoma |
| 1388 | TACCCTGCAGAACCGCATCTGT | prostate adenocarcinoma |
| 1389 | TACCTGCTCTACACAACTTTGT | prostate adenocarcinoma |
| 1390 | TAGCTGCCAGTTTCATAACTGT | prostate adenocarcinoma |
| 1391 | TAGGTGATCAGACTCATGTTGA | prostate adenocarcinoma |
| 1392 | TATGTGTGACTGACAGGTATTTTCTGAGC | prostate adenocarcinoma |
| 1393 | TATTTTATCAGAGTGATCTTGA | prostate adenocarcinoma |
| 1394 | TCAATGCACTCCCGAACTTTGT | prostate adenocarcinoma |
| 1395 | TCAGTTATCAGACTGACGTITA | prostate adenocarcinoma |
| 1396 | TCCCTAAGACCCTCACTTGTGA | prostate adenocarcinoma |
| 1397 | TCCTTGGTGGTCTAGTGGTTAGTATTCGGC | prostate adenocarcinoma |
| 1398 | TCGGTTCACTACAGAACTGTGT | prostate adenocarcinoma |
| 1399 | TCGTGTTTAGTACTCTGCGTT | prostate adenocarcinoma |
| 1400 | TCTCCCAACCCTTGTACAATT | prostate adenocarcinoma |
| 1401 | TCTCTTATCAGATTGATGTTG | prostate adenocarcinoma |
| 1402 | TCTGAGATTTCAACTTAACTTGACCG | prostate adenocarcinoma |
| 1403 | TCTGGCACTGGTAGAAGTCACT | prostate adenocarcinoma |
| 1404 | TGAGATGAAGCACCCTACCTCT | prostate adenocarcinoma |
| 1405 | TGAGATGAAGTAGTGTACCT | prostate adenocarcinoma |
| 1406 | TGAGATGTAACACTGTTGCTCT | prostate adenocarcinoma |
| 1407 | TGTAAACATCCTGGACTCGAA | prostate adenocarcinoma |
| 1408 | TTACATGAAGCAGTGTACCTCT | prostate adenocarcinoma |
| 1409 | TTAGCACTGGTATAATTCACTG | prostate adenocarcinoma |
| 1410 | TTAGGACTGAGGCCTTCTTATT | prostate adenocarcinoma |
| 1411 | TTAGTGCACTTCAAAACTTTGT | prostate adenocarcinoma |
| 1412 | TTGATCGTTCGGCTAGCGTGA | prostate adenocarcinoma |
| 1413 | TTGCTTATTAGAGTGATGTTGA | prostate adenocarcinoma |
| 1414 | TTTCGCACTTGTCCAGGCCTGT | prostate adenocarcinoma |
| 1415 | TTTGCACTGGTATAATTCACTGT | prostate adenocarcinoma |
| 1416 | TTTGGCAATGGTAGAATTCACT | prostate adenocarcinoma |
| 1417 | TTTGGCAATGGTCGAACTCTCA | prostate adenocarcinoma |
| 1418 | TTTGGCACTAGCCCATTTTTG | prostate adenocarcinoma, thyroid carcinoma |
| 1419 | TTTGTTAGTACGGCTCGCGTGA | prostate adenocarcinoma |
| 1420 | TTTGTTATCAGAGTGATGTTGAC | prostate adenocarcinoma |
| 1421 | TTTGTTCGTACGGCTCGTGTGA | prostate adenocarcinoma |
| 1422 | TTTGTTCGTTCGTCTCTGGTG | prostate adenocarcinoma |
| 1423 | TTTGTTCGTTCGTCTCTGGTGT | prostate adenocarcinoma |
| 1424 | CATAAGGAAAGGTTAAAAAAAGTAAAAGG | adrenocortical carcinoma, rectum |
| | | adenocarcinoma |
| 1425 | ATGGCACTGGTAGAATTCACTGAAT | adrenocortical carcinoma, rectum |
| | | adenocarcinoma |
| 1426 | AGAGAAGGGTCGGGGCGGCAGG | cervical squamous cell carcinoma and |
| | | endocervical adenocarcinoma, rectum |
| | | adenocarcinoma |
| 1427 | GATCCCGAGGCCTCTCC | cervical squamous cell carcinoma and |
| | | endocervical adenocarcinoma, rectum |
| | | adenocarcinoma |
| 1428 | GGCCGAAGTGGAGAAGGGTTCCATGTGAAC | cervical squamous cell carcinoma and |
| | | endocervical adenocarcinoma, rectum |
| | | adenocarcinoma |
| 1429 | GGGAAGGCCCGGCGGGGAAGGG | cervical squamous cell carcinoma and |
| | | endocervical adenocarcinoma, rectum |
| | | adenocarcinoma |
| 1430 | GTTCCTCCCGGGGCTACGCCTGTCT | cervical squamous cell carcinoma and |
| | | endocervical adenocarcinoma, rectum |
| | | adenocarcinoma |
| 1431 | CGCAATGAAGGTGAAGGCCG | head and neck squamous cell carcinoma, rectum |
| | | adenocarcinoma |
| 1432 | CTCCACGCGCCCCGCG | head and neck squamous cell carcinoma, rectum |
| | | adenocarcinoma |
| 1433 | GGGTCCGGTGCGGAGTG | head and neck squamous cell carcinoma, rectum |
| | | adenocarcinoma |
| 1434 | GGTTCCGGCGGCGTCCGGT | head and neck squamous cell carcinoma, rectum |
| | | adenocarcinoma |
| 1435 | TAGCGACGTCGCTTTTT | head and neck squamous cell carcinoma, rectum |
| | | adenocarcinoma |
| 1436 | AAAGAACTTTTGACTGGAGAG | rectum adenocarcinoma |
| 1437 | AAAGCAGAATCAATGTCTTTTCC | rectum adenocarcinoma |
| 1438 | AACAGATCGTCACTCGGTG | rectum adenocarcinoma |
| 1439 | AAGACCCTCTGGCTGTGGACAA | rectum adenocarcinoma |
| 1440 | AAGCTGGGTTGAGAG | rectum adenocarcinoma |
| 1441 | AATAATAGGACAGTAG | rectum adenocarcinoma |
| 1442 | ACACAGTATGGGTGCTGCCTCTG | rectum adenocarcinoma |
| 1443 | ACCGTCGTAGTTCCGAC | rectum adenocarcinoma |
| 1444 | ACGCATGGGTTTGGATTTATGATGGGCTGG | rectum adenocarcinoma |
| 1445 | ACGCGATGTGATTTCTGCCCAGTGC | rectum adenocarcinoma |
| 1446 | ACGTGGGCACATTACCCCTCTGACC | rectum adenocarcinoma |
| 1447 | AGAAAAGACTCTGACCAG | rectum adenocarcinoma |
| 1448 | AGATCGTCACTCGGTG | rectum adenocarcinoma |
| 1449 | AGGATGCTGTAGCCTTAACC | rectum adenocarcinoma |
| 1450 | AGGGAACGTGAGCTGGGTTTAGACCGTCGT | rectum adenocarcinoma |
| 1451 | AGGGCAAAAGCTCGCTTGATCTTGATCGT | rectum adenocarcinoma |
| 1452 | AGGTGCAGATCTTGGTGGTCGT | rectum adenocarcinoma |
| 1453 | AGTTGTGTGGGCTGTAGAG | rectum adenocarcinoma |
| 1454 | AGTTTGATCTCAGACTG | rectum adenocarcinoma |
| 1455 | ATACTGCCTGGTCATGATGA | rectum adenocarcinoma |
| 1456 | ATGGCACTGCTAGAATTCACTGT | rectum adenocarcinoma |
| 1457 | ATGGTCAGTAGGACAGAAGGTA | rectum adenocarcinoma |
| 1458 | ATTAACATGCTGAGGATTGTAGAGC | rectum adenocarcinoma |
| 1459 | ATTGGTCGTGGTTGTAGTGG | rectum adenocarcinoma |
| 1460 | CAGGATTGACAGCTTGATAGCTCT | rectum adenocarcinoma |
| 1461 | CAGTGGTAGAGCCCA | rectum adenocarcinoma |
| 1462 | CCAGAGGTCGATGGATCGTAACC | rectum adenocarcinoma |
| 1463 | CCCACTGCTTCACTTGACTCGA | rectum adenocarcinoma |
| 1464 | CCCCCGAGACAGAGTTTCA | rectum adenocarcinoma |
| 1465 | CCCCTCCTCCTCGGC | rectum adenocarcinoma |
| 1466 | CCCCTCTCCGGAGTCCG | rectum adenocarcinoma |
| 1467 | CCCTGAGTGACAGAACCG | rectum adenocarcinoma |
| 1468 | CCGCGGAGCCTCGGTTGGCCTCGGATAG | rectum adenocarcinoma |
| 1469 | CCGGCTGTGGAATCTGAAGACA | rectum adenocarcinoma |
| 1470 | CGCCTGTCACGCGGGAGACCG | rectum adenocarcinoma |
| 1471 | CGCTACGCCGCGACGAGGAGG | rectum adenocarcinoma |
| 1472 | CGGAAAAAAGGACTTTT | rectum adenocarcinoma |
| 1473 | CGGTTCCGGCGGCGTCCGGTGAGCT | rectum adenocarcinoma |
| 1474 | CGTCGTCGGCGGTGGGGGCGTGTT | rectum adenocarcinoma |
| 1475 | CGTGGGGAACCTGCCGCTAAACCATTCGT | rectum adenocarcinoma |
| 1476 | CTCCATTATGAGCACTTAAAGT | rectum adenocarcinoma |
| 1477 | CTCGGGAGGGCGCGC | rectum adenocarcinoma |
| 1478 | CTCTACCTTACCTACC | rectum adenocarcinoma |
| 1479 | CTCTGTGTCCCCACCCAA | rectum adenocarcinoma |
| 1480 | CTGCCCCCTCTGCTGATGCC | rectum adenocarcinoma |
| 1481 | CTGTAGTGTAGTGGTCATC | rectum adenocarcinoma |
| 1482 | CTGTCGGGGCACCTGGAG | rectum adenocarcinoma |
| 1483 | CTGTGAACCTGAGACTCAGGACGCA | rectum adenocarcinoma |
| 1484 | CTGTGCCTGGCCTGAGGGGC | rectum adenocarcinoma |
| 1485 | CTGTGCTCTCAGGAAGG | rectum adenocarcinoma |
| 1486 | CTTAAAGGAATTGACGGAAGGGCACC | rectum adenocarcinoma |
| 1487 | CTTCCACAGCTACTGGTCTGCAG | rectum adenocarcinoma |
| 1488 | GAACGTGAGCTGGGTTAGACCGT | rectum adenocarcinoma |
| 1489 | GAAGAGGGTTACACAGTG | rectum adenocarcinoma |
| 1490 | GAATCCCGCCCAGGCGGAACGATACGGC | rectum adenocarcinoma |
| 1491 | GACGAGGTGGCCGGGTGGTTAAGG | rectum adenocarcinoma |
| 1492 | GAGATCAGGGACATGTTGCT | rectum adenocarcinoma |
| 1493 | GAGGAATTGCTTTGACTTCTGAC | rectum adenocarcinoma |
| 1494 | GAGGGTCCAGGGTTCAGGTCCCT | rectum adenocarcinoma |
| 1495 | GAGTTGGTGGTTCAGTGGTAGAA | rectum adenocarcinoma |
| 1496 | GAGTTTAAGTCTTGCCTG | rectum adenocarcinoma |
| 1497 | GATGTTGTAGCTTATCAGACT | rectum adenocarcinoma |
| 1498 | GATTCTGAGCAGATTCGTCTAGG | rectum adenocarcinoma |
| 1499 | GATTGTGGGTTCGGGTCCCCTC | rectum adenocarcinoma |
| 1500 | GCACCACCAGGAGTGGAGCC | rectum adenocarcinoma |
| 1501 | GCAGGTGTCCTAAGGCGAGCTCAGGGAGG | rectum adenocarcinoma |
| 1502 | GCATTGGTGGTTCAGTGGTCGGA | rectum adenocarcinoma |
| 1503 | GCCGAATAGGAACAGCTCCGG | rectum adenocarcinoma |
| 1504 | GCGGCGGCCGGCGGGCGGTGG | rectum adenocarcinoma |
| 1505 | GCGGCGGCGGCGAATCTGGACGCGAGCC | rectum adenocarcinoma |
| 1506 | GCGTGTGGTGTGCGTC | rectum adenocarcinoma |
| 1507 | GCTCCCCGTCCTCCCCCCCCC | rectum adenocarcinoma |
| 1508 | GCTGACCCCCTTCGCG | rectum adenocarcinoma |
| 1509 | GCTTACAGTGCAGGTAGA | rectum adenocarcinoma |
| 1510 | GCTTGGCTTGCATGAGGTCCCGG | rectum adenocarcinoma |
| 1511 | GGAGAGAAGGGTCGGGGCGGCA | rectum adenocarcinoma |
| 1512 | GGCCCCCCCCGCGGGGA | rectum adenocarcinoma |
| 1513 | GGCCGGTTAGCTCAGTTGGTGAGA | rectum adenocarcinoma |
| 1514 | GGCGCCCTGGAATGG | rectum adenocarcinoma |
| 1515 | GGTCGCAGTCTCCCTTG | rectum adenocarcinoma |
| 1516 | GGTCGTCCCGCTCCGGC | rectum adenocarcinoma |
| 1517 | GGTTCGGACCCCGTC | rectum adenocarcinoma |
| 1518 | GTCAGCCCCTCTCCG | rectum adenocarcinoma |
| 1519 | GTGATCGTATAGTGGTTAGTACTCCG | rectum adenocarcinoma |
| 1520 | GTGATCGTATAGTGGTTAGTACTCTGTCTC | rectum adenocarcinoma |
| 1521 | GTGATCGTATAGTGTTAGTA | rectum adenocarcinoma |
| 1522 | GTGGACTCGACTGTAAGGAC | rectum adenocarcinoma |
| 1523 | GTTCGTATCCCACCTTCGTCG | rectum adenocarcinoma |
| 1524 | GTTTCCCAGATTTCAGGA | rectum adenocarcinoma |
| 1525 | TAATACTGCCTGGTAATGATAACT | rectum adenocarcinoma |
| 1526 | TAGAATTTGTCTAGTAGGCTTTCTGG | rectum adenocarcinoma |
| 1527 | TATAACTGAACTCCTCACACCCAAT | rectum adenocarcinoma |
| 1528 | TCACCTTGTATAGCTGATAAA | rectum adenocarcinoma |
| 1529 | TCAGAAGGCTGCGTG | rectum adenocarcinoma |
| 1530 | TGAAATGNTTAGGACCACTAGA | rectum adenocarcinoma |
| 1531 | TGACATTTTAAACCACCAAGATCGCTGATG | rectum adenocarcinoma |
| 1532 | TGAGATCAGGGACATGTTGCT | rectum adenocarcinoma |
| 1533 | TGCACTACAGAACTT | rectum adenocarcinoma |
| 1534 | TGCATCACAGAACTTTG | rectum adenocarcinoma |
| 1535 | TGCATGCATTTATCAGAT | rectum adenocarcinoma |
| 1536 | TGGATTGGAAATCCATTGGGG | rectum adenocarcinoma |
| 1537 | TGGATTTTTGGAACTAGGAG | rectum adenocarcinoma |
| 1538 | TGGCTCTAAGGGCTGGGTCGGTCGC | rectum adenocarcinoma |
| 1539 | TGTAAACATCCCCGACTGGCAGCTA | rectum adenocarcinoma |
| 1540 | TGTAATAGGAACTCAACAAGCT | rectum adenocarcinoma |
| 1541 | TGTCATGGACTCTGGAGAC | rectum adenocarcinoma |
| 1542 | TTGGAGAACGTGGACACC | rectum adenocarcinoma |
| 1543 | TTGTAGTGTGCTGATGTGCA | rectum adenocarcinoma |

TABLE X
Table containing the number of unique oncRNA species
(under column head “oncRNA counts”) found significantly
present within cancer samples (under column head “Cancer”) and
not in normal tissue samples for each of the 32 cancer
types from The Cancer Genome Atlas (TCGA).

| | oncRNA | Log10 | | Log10 |
|---|---|---|---|---|
| Cancer | Counts | Counts | Prefilter | Prefilter |
| SKCM | 106232 | 5.026255 | 109219 | 5.038298 |
| TGCT | 100731 | 5.003163 | 103619 | 5.015439 |
| THYM | 98941 | 4.995376 | 101437 | 5.006196 |
| HNSC | 87619 | 4.942598 | 89945 | 4.953977 |
| UCEC | 79838 | 4.90221 | 82340 | 4.915611 |
| OV | 79657 | 4.901224 | 81810 | 4.912806 |
| LUAD | 69668 | 4.843033 | 71280 | 4.852968 |
| CESC | 68965 | 4.838629 | 70867 | 4.850444 |
| DLBC | 65908 | 4.818938 | 68011 | 4.832579 |
| BLCA | 64899 | 4.812238 | 66475 | 4.822658 |
| UVM | 59760 | 4.776411 | 61300 | 4.78746 |
| ACC | 56884 | 4.75499 | 58515 | 4.767267 |
| LUSC | 56157 | 4.749404 | 57659 | 4.760867 |
| COAD | 50835 | 4.706163 | 52449 | 4.719737 |
| READ | 41690 | 4.620032 | 43080 | 4.634276 |
| THCA | 39391 | 4.595397 | 40276 | 4.605046 |
| LIHC | 38214 | 4.582222 | 39232 | 4.59364 |
| ESCA | 37529 | 4.574367 | 38492 | 4.58537 |
| STAD | 36341 | 4.560397 | 37234 | 4.57094 |
| PAAD | 33362 | 4.523252 | 33780 | 4.52866 |
| LGG | 31804 | 4.502482 | 32275 | 4.508866 |
| PCPG | 29850 | 4.474944 | 30468 | 4.483844 |
| CHOL | 27028 | 4.431814 | 27477 | 4.438969 |
| MESO | 26201 | 4.418318 | 26754 | 4.427389 |
| UCS | 25002 | 4.397975 | 25917 | 4.413585 |
| KIRP | 24351 | 4.386517 | 24810 | 4.394627 |
| KICH | 22285 | 4.348013 | 22772 | 4.357401 |
| SARC | 21744 | 4.337339 | 22095 | 4.344294 |
| BRCA | 16515 | 4.217879 | 17045 | 4.231597 |
| LAML | 9598 | 3.982181 | 10098 | 4.004235 |
| PRAD | 8706 | 3.939819 | 8812 | 3.945074 |
| KIRC | 3067 | 3.486714 | 3153 | 3.498724 |

The procedure above was preformed again using a bigger sample set. All of the annotated oncRNAs found across all the cancer types in TCGA database. Annotated oncRNAs found significantly in the following TCGA cancer types: Rectum adenocarcinoma (READ), Colon adenocarcinoma (COAD), Esophageal carcinoma (ESCA), Head and Neck squamous cell carcinoma (HNSC), Lymphoid Neoplasm Diffuse Large B-cell Lymphoma (DLBC), Liver hepatocellular carcinoma (LIHC), Lung adenocarcinoma (LUAD), Lung squamous cell carcinoma (LUSC), Ovarian serous cystadenocarcinoma (OV), Pancreatic adenocarcinoma (PAAD), Stomach adenocarcinoma (STAD), and Breast invasive carcinoma (BRCA).

Bar plots of the log 10 number of oncRNAs found to be significantly present in each of 32 TCGA cancer types in comparison to TCGA normal samples were produce. Fisher exact test was used to statistically test for significance and an FDR cutoff of 0.1 was used to correct for multiple hypothesis testing as described above.

Plots of pan-cancer classifier performance accuracy on 32 TCGA cancer types across 5 folds of cross-validation on the training dataset (80/20 split) versus number of features used to train. Experiment was run for recursive feature elimination purposes to select most informative features to train our final model. Features used were the binary encoding of presence/absence of oncRNAs within samples. An Extreme Gradient Boosting (XGB) model with 500 estimators and max depth of 3 was used as our selection model for this feature selection step.

Plost of pan-cancer classifier performance accuracy on 32 TCGA cancer types across 5 folds of cross-validation on the training dataset (from a 80/20 train/test split) versus number of features used to train. Experiments were run for recursive feature elimination purposes to select most informative features to train our final model. Features used were the counts per million (c.p.m) of oncRNAs within samples. An Extreme Gradient Boosting (XGB) model with 500 estimators and max depth of 3 was used as our selection model for this feature selection step. Heatmaps of feature selected oncRNAs via recursive feature elimination used to train our final XGB classifier to classify cancer types across 32 cancer types in TCGA. Features used were the binary encoding of presence/absence of oncRNAs within samples.

Additional heatmaps of feature selected oncRNAs were created via recursive feature elimination used to train our final XGB classifier to classify cancer types across 32 cancer types in TCGA. Features used were the counts per million (c.p.m) of oncRNAs within samples. Confusion matrices were created of our final pan-cancer XGB performance and cancer subtypes on held out test data from an 80/20 train/test, class-balanced split of 10403 cancer samples. Confusion matrix was normalized by columns. Classifier was trained on binary representations (presence/absence) of feature selected oncRNAs. Final XGB classifier consisted of 500 estimators, max depth of 3, and a lambda regularization parameter of 8.

ROC curves of Gradient Boosting Classifiers (GBC) trained to make cancer-specific binary predictions using a one-vs-the-rest multiclass strategy was employed. GBCs were trained on training data from an 80/20 train/test, class-balanced split and evaluated on the held-out test set. Features used were the binary encodings from previously selected oncRNAs. ROC curves of the following cancer classifiers were plotted for: BRCA, COAD, ESCA, LIHC, LUAD, LUSC, OV, PAAD, and STAD.

Classification results of classifiers trained for cancer-normal predictions for each of 32 TCGA cancer types. For each cancer, a linear support vector classification model with Li regularization was trained on samples specific to the cancer and normal samples. 80/20 train/test split was done to separate the dataset of cancer-specific samples and all TCGA normal samples into training and testing datasets.

ROC curves of linear support vector classification models used to make cancer-normal predictions. Overall accuracy on the test dataset (80/20 train/test split) is also generated for the following cancer types: BRCA, COAD, ESCA, LIHC<LUAD, LUSC, OV, PAAD, and STAD.

### BRCA Tumors

Violin plots and box plots of oncRNA expression across normal, stage I, and stage II and III samples in the TCGA-BRCA dataset. P values were calculated and FDR corrected using a one-way ANOVA test for associations. Normal, n=104, T1: n=277, T2: n=628, T3: n=137.

Violin plots and box plots of oncRNA expression across samples grouped by BRCA subtypes in the TCGA-BRCA dataset. P values were calculated and FDR corrected using a one-way ANOVA test for associations. BRCA-Basal, n=172, BRCA-Her2: n=77, BRCA-LumA: n=505, BRCA-LumB: n=192.

A confusion matrix was created showing the test performance of an XGB classifier trained on BRCA oncRNAs to predict BRCA subtypes. 946 BRCA patient samples with annotated subtypes were split into train/test sets using an 80/20 ratio. A one-way ANOVA test was first used to feature select a final list of 682 BRCA oncRNAs on the training dataset. An XGB classifier with 100 estimators and a max depth of 3 was then trained the selected features and evaluated on the held-out test dataset.

Heatmaps of the presence/absence of 1062 BRCA oncRNAs demonstrate significant differences in expression across BRCA subtype groups: Basal, Her2, LumA, and LumB. BRCA oncRNAs were tested using a one-way ANOVA test. FDR correction with a cutoff of 0.1 was set to correct for multiple hypothesis testing.

Heatmap of the c.p.m expression of 1062 BRCA oncRNAs demonstrate significant differences in expression across BRCA subtype groups: Basal, Her2, LumA, and LumB. BRCA oncRNAs were tested using a one-way ANOVA test. FDR correction with a cutoff of 0.1 was set to correct for multiple hypothesis testing.

Survival analysis for TCGA-BRCA patients was stratified into groups of high and low risk based on presence and absence of oncRNAs. Risk scores was calculated using a regularized cox model (Cox Elastic Net) fit on the training dataset with cross-validation and evaluated on a held-out dataset (80/20 train/test split of 1068 patients). Survival curves of patients in the held-out dataset are shown. P value was calculated from a log-rank test.

Heatmaps were constructed of the signs of the regression coefficients from a regularized cox model across BRCA patients in the held-out dataset. A positive coefficient for an oncRNA was plotted with a value of 1 (−1 for negative). Positive coefficients indicate contribution towards higher risk and negative towards lower risk.

Additional heatmap were constructed of reconstructed gene expression in held-out BRCA patient dataset. An autoencoder was first trained to generate a reduced embedding of gene expression profiles (of 589 genes with highest variance) across all BRCA patients. BRCA patient oncRNA binary profiles were then used to train a fully connected neural network to predict gene expression profile embeddings. The decoder from previously trained autoencoder were then used to reconstruct gene expression profiles from predicted gene expression embeddings. oncRNA neural network was trained the training data generated via 80/20 split from 1085 BRCA patient data.

Lengthy table referenced here
US20240150829A1-20240509-T00001
Please refer to the end of the specification for access instructions.

### REFERENCES

1. S. F. Tavazoie et al., Endogenous human microRNAs that suppress breast cancer metastasis. Nature. 451, 147-U3 (2008).
2. C. J. David, M. Chen, M. Assanah, P. Canoll, J. L. Manley, HnRNP proteins controlled by c-Myc deregulate pyruvate kinase mRNA splicing in cancer. Nature. 463, 364-368 (2010).
3. S. Vanharanta et al., Loss of the multifunctional RNA-binding protein RBM47 as a source of selectable metastatic traits in breast cancer. eLife. 3 (2014), doi:10.7554/eLife.02734.
4. L. Fish et al., Muscleblind-like 1 suppresses breast cancer metastatic colonization and stabilizes metastasis suppressor transcripts. Genes Dev. 30, 386-398 (2016).
5. L.-Y. Chen, J. Lingner, AUF1/HnRNP D RNA binding protein functions in telomere maintenance. Mol. Cell. 47, 1-2 (2012).
6. H. Goodarzi et al., Modulated expression of specific tRNAs drives gene expression and cancer progression. Cell. 165, 1416-1427 (2016).
7. H. Goodarzi et al., Endogenous tRNA-Derived Fragments Suppress Breast Cancer Progression via YBX1 Displacement. Cell. 161, 790-802 (2015).
8. D. K. Simanshu, D. V. Nissley, F. McCormick, RAS Proteins and Their Regulators in Human Disease. Cell. 170, 17-33 (2017).
9. R. Ren, Mechanisms of BCR-ABL in the pathogenesis of chronic myelogenous leukaemia. Nat. Rev. Cancer. 5, 172-183 (2005).
10. R.-K. Lin, Y.-C. Wang, Dysregulated transcriptional and post-translational control of DNA methyltransferases in cancer. Cell Biosci. 4, 46 (2014).
11. A. A. Alizadeh et al., Toward understanding and exploiting tumor heterogeneity. Nat. Med. 21, 846-853 (2015).
12. A. Nguyen, M. Yoshida, H. Goodarzi, S. F. Tavazoie, Highly variable cancer subpopulations that exhibit enhanced transcriptome variability and metastatic fitness. Nat. Commun. 7, 11246 (2016).
13. R. J. Taft, K. C. Pang, T. R. Mercer, M. Dinger, J. S. Mattick, Non-coding RNAs: Regulators of disease. J. Pathol. 220 (2010), pp. 126-139.
14. M. Esteller, Non-coding RNAs in human disease. Nat. Rev. Genet. 12, 861-874 (2011).
15. A. J. Minn et al., Distinct organ-specific metastatic potential of individual breast cancer cells and primary tumors. J. Clin. Invest. 115, 44-55 (2005).
16. J. M. Loo et al., Extracellular Metabolic Energetics Can Promote Cancer Progression. Cell. 160, 393-406 (2015).
17. D. N. Cooper, L. P. Berg, V. V Kakkar, J. Reiss, Ectopic (illegitimate) transcription: new possibilities for the analysis and diagnosis of human genetic disease. Ann. Med. 26, 9-14 (1994).
18. A. A. Margolin et al., ARACNE: An Algorithm for the Reconstruction of Gene Regulatory Networks in a Mammalian Cellular Context. BMC Bioinformatics. 7, S7 (2006).
19. H. Goodarzi et al., Metastasis-suppressor transcript destabilization through TARBP2 binding of mRNA hairpins. Nature (2014), doi:10.1038/nature13466.
20. B. Kim, K. Jeong, V. N. Kim, Genome-wide Mapping of DROSHA Cleavage Sites on Primary MicroRNAs and Noncanonical Substrates. Mol. Cell. 66, 258-269.e5 (2017).
21. D. Ray et al., A compendium of RNA-binding motifs for decoding gene regulation. Nature. 499, 172-177 (2013).
22. Y.-C. T. Yang et al., CLIPdb: a CLIP-seq database for protein-RNA interactions. BMC Genomics. 16, 51 (2015).
23. E. L. Van Nostrand et al., Robust transcriptome-wide discovery of RNA-binding protein binding sites with enhanced CLIP (eCLIP). Nat. Methods. 13, 508-514 (2016).
24. H. Goodarzi et al., Systematic discovery of structural elements governing stability of mammalian messenger RNAs. Nature. 485, 264-268 (2012).
25. S. Memczak et al., Circular RNAs are a large class of animal RNAs with regulatory potency. Nature. 495, 333-8 (2013).
26. P. Sumazin et al., An extensive microRNA-mediated network of RNA-RNA interactions regulates established oncogenic pathways in glioblastoma. Cell. 147, 370-381 (2011).
27. A. Helwak, G. Kudla, T. Dudnakova, D. Tollervey, Mapping the human small non-coding RNA interactome by CLASH reveals frequent noncanonical binding. Cell. 153, 654-65 (2013).
28. T. Fiskaa et al., Distinct Small RNA Signatures in Extracellular Vesicles Derived from Breast Cancer Cell Lines. PLoS ONE. 11 (2016), doi:10.1371/journal.pone.0161824.
29. W. Zhou et al., Cancer-secreted miR-105 destroys vascular endothelial barriers to promote metastasis. Cancer Cell. 25, 501-515 (2014).
30. S. K. Chakrabortty, A. Prakash, G. Nechooshtan, S. Hearn, T. R. Gingeras, Extracellular vesicle-mediated transfer of processed and functional RNY5 RNA. RNA N. Y. N. 21, 1966-1979 (2015).
31. X. Wu et al., De novo sequencing of circulating small non-coding RNAs identifies novel markers predicting clinical outcome of locally advanced breast cancer. J. Transl. Med. 10, 42-42 (2012).
32. M. D. Giraldez et al., Accuracy, Reproducibility And Bias Of Next Generation Sequencing For Quantitative Small RNA Profiling: A Multiple Protocol Study Across Multiple Laboratories. bioRxiv, 113050 (2017).
33. O. Elemento, N. Slonim, S. Tavazoie, A universal framework for regulatory element discovery across all Genomes and data types. Mol. Cell. 28, 337-350 (2007).

LENGTHY TABLES
The patent application contains a lengthy table section. A copy of the table is available in electronic form from the USPTO web site (https://seqdata.uspto.gov/?pageRequest=docDetail&DocID=US20240150829A1). An electronic copy of the table will also be available from the USPTO upon request and payment of the fee set forth in 37 CFR 1.19(b)(3).

1.-69. (canceled)

70. A method for sequencing a ribonucleic acid, comprising:
(a) providing the ribonucleic acid sequence or a functional fragment thereof;
(b) subjecting the ribonucleic acid or the functional fragment thereof to reverse transcription using a reverse transcriptase and a primer to generate a complementary deoxyribonucleic acid (cDNA) molecule, wherein the cDNA molecule comprises a sequence corresponding to a target sequence of the ribonucleic acid or the functional fragment thereof;
(c) amplifying the ribonucleic acid or the functional fragment thereof using one or more probes having different single stranded versus double stranded character; and
(d) sequencing the cDNA molecule or a derivative thereof.

71. The method of claim 70, wherein the ribonucleic acid sequence or the functional fragment thereof has a length of less than about 200 nucleotides.

72. The method of claim 70, wherein the ribonucleic acid sequence or the functional fragment thereof has a length of less between 50 and 100 nucleotides.

73. The method of claim 70, further comprising, after (b), amplifying the cDNA molecule.

74. The method of claim 70, wherein (c) comprises a Polymerase Chain Reaction (PCR).

75. The method of claim 70, wherein (c) occurs subsequent to (b).

76. The method of claim 70, wherein (c) comprises rolling circle amplification.

77. The method of claim 70, wherein (c) comprises a ligase chain reaction, multiplex ligatable probe amplification, in vitro transcription (IVT), strand displacement amplification, transcription-mediated amplification, or RNA (Eberwine) amplification.

78. A reaction mixture, comprising:
a ribonucleic acid sequence or a functional fragment thereof;
one or more proves having different single stranded versus double stranded character for amplification of the ribonucleic acid or the functional fragment thereof;
a reverse transcriptase;
one or more primers; and
a complementary deoxyribonucleic acid molecule wherein the cDNA molecule comprises a sequence corresponding to a target sequence of the ribonucleic acid or the functional fragment thereof.

79. The reaction mixture of claim 78, wherein the ribonucleic acid sequence or the functional fragment thereof has a length of less than about 200 nucleotides.

80. The reaction mixture of claim 78, wherein the ribonucleic acid sequence or the functional fragment thereof has a length of less between 50 and 100 nucleotides.

81. The reaction mixture of claim 78, further comprising a single stranded oligonucleotide containing one or more universal primer sequences.

82. The reaction mixture of claim 78, further comprising one or more polyadenylated sequences.

83. The reaction mixture of claim 78, further comprising one or more adapter sequences.

84. The reaction mixture of claim 78, wherein the one or more primers comprises a primer complementary to a universal primer sequence.

85. The reaction mixture of claim 78, wherein the one or more primers comprises a poly(T) primer.

86. The reaction mixture of claim 83, wherein the one or more primers comprises a primer complementary to an adapter sequence of the one or more adapter sequences.

87. The reaction mixture of claim 78, wherein the one or more primers comprises an amplification primer that specifically binds to the ribonucleic acid or the functional fragment thereof.

88. The reaction mixture of claim 78, wherein the one or more primers comprises a reverse transcription primer.
